SEQUENCE LISTING (471 sequences)

SEQ ID NO: 1 - protein, 31 aa, synthetic construct
  MOD_RES 31: note = AminoEthoxyEthoxyAcetic Acyl-MaleimidoPropionic Acyl
  SITE 2: note = D-alanine
HAEGTFTSDVSSYLEGQAAKEFIAWLVKGRK

SEQ ID NO: 2 - protein, 40 aa, synthetic construct
  MOD_RES 40: note = AminoEthoxyEthoxyAcetic Acyl-MaleimidoPropionic Acyl
HGEGTFTSDLSKQMEEEAVRLFIEWLKNGGPSSGAPPPSK

SEQ ID NO: 3 - protein, 39 aa, synthetic construct
HGEGTFTSDLSKQMEEEAVRLFIEWLKNGGPSSGAPPPS

SEQ ID NO: 4 - protein, 30 aa, synthetic construct
HAEGTFTSDVSSYLEGQAAKEFIAWLVKGR

SEQ ID NO: 5 - protein, 42 aa, synthetic construct
YAEGTFISDYSIAMDKIHQQDFVNWLLAQKGKKNDWKHNITQ

SEQ ID NO: 6 - protein, 29 aa, synthetic construct
HSQGTFTSDYSKYLDSRRAQDFVQWLMNT

SEQ ID NO: 7 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDVSSYLEGQAAKEFIAWLVNGGPSSGAPPPSK

SEQ ID NO: 8 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFIAWLVKGRPSSGAPPPSK

SEQ ID NO: 9 - protein, 39 aa, synthetic construct
  MOD_RES 32: note = AminoEthoxyEthoxyAcetic Acyl-MaleimidoPropionic Acyl
HGEGTFTSDLSKQMEEEAVRLFIEWLKNGGPKSGAPPPS

SEQ ID NO: 10 - protein, 39 aa, synthetic construct
  SITE 2: note = D-alanine
  MOD_RES 12: note = AminoEthoxyEthoxyAcetic Acyl-MaleimidoPropionic Acyl
HAEGTFTSDVSKYLEGQAAKEFIAWLVKGRPSSGAPPPS

SEQ ID NO: 11 - protein, 39 aa, synthetic construct
  MOD_RES 16: note = AminoEthoxyEthoxyAcetic Acyl-MaleimidoPropionic Acyl
  SITE 2: note = D-alanine
HAEGTFTSDVSSYLEKQAAKEFIAWLVKGRPSSGAPPPS

SEQ ID NO: 12 - protein, 39 aa, synthetic construct; identical to SEQ ID NO: 9
  MOD_RES 32: note = AminoEthoxyEthoxyAcetic Acyl-MaleimidoPropionic Acyl

SEQ ID NO: 13 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDINKVLDTIAAKEFIAWLVKGRPSSGAPPPSK

SEQ ID NO: 14 - protein, 40 aa, synthetic construct
  MOD_RES 40: note = Palmitic acyl // Hexadecanoic acyl
  MOD_RES 2: note = Aib
  MOD_RES 20: note = Aib
YXEGTFTSDYSIYLDKQAAXEFVQWLLAGGPSSGAPPPSK

SEQ ID NO: 15 - protein, 40 aa, synthetic construct
  MOD_RES 40: note = AminoEthoxyEthoxyAcetic Acyl-MaleimidoPropionic Acyl
  SITE 30: note = D-alanine
YGGGTFTSDSFFYLELSHAKDFINWLQLGAPSSGAPPPSK

SEQ ID NO: 16 - protein, 40 aa, synthetic construct
  MOD_RES 2..3: note = Aib
  MOD_RES 20: note = Aib
  MOD_RES 28: note = Aib
  MOD_RES 40: note = AminoEthoxyEthoxyAcetic Acyl-MaleimidoPropionic Acyl
HXXGTFTSDEMNYLDDWMQXAFVNWLVXGIPSSGAPPPSK

SEQ ID NO: 17 - protein, 40 aa, synthetic construct
  MOD_RES 2: note = Aib
  MOD_RES 40: note = AminoEthoxyEthoxyAcetic Acyl-MaleimidoPropionic Acyl
FXPGTFTSDGHNYLDWQDAKEFIQWLGWGVPSSGAPPPSK

SEQ ID NO: 18 - protein, 40 aa, synthetic construct
  MOD_RES 2: note = Aib
  MOD_RES 13: note = Aib
  MOD_RES 20: note = Aib
  MOD_RES 40: note = AminoEthoxyEthoxyAcetic Acyl-MaleimidoPropionic Acyl
YXMGTFTSDPQIXLEMKEQXDFINWLNDGFPSSGAPPPSK

SEQ ID NO: 19 - protein, 40 aa, synthetic construct
  SITE 2: note = D-alanine
  MOD_RES 13: note = Aib
  MOD_RES 20: note = Aib
  MOD_RES 40: note = AminoEthoxyEthoxyAcetic Acyl-MaleimidoPropionic Acyl
HAPGTFTSDNHEXLDTYRQXDFINWLDTGVPSSGAPPPSK

SEQ ID NO: 20 - protein, 40 aa, synthetic construct
  SITE 2: note = D-alanine
  MOD_RES 40: note = AminoEthoxyEthoxyAcetic Acyl-MaleimidoPropionic Acyl
YANGTFTSDIWAYLDSSFAQDFVAWLYIGKPSSGAPPPSK

SEQ ID NO: 21 - protein, 40 aa, synthetic construct
  MOD_RES 2: note = Aib
  MOD_RES 40: note = AminoEthoxyEthoxyAcetic Acyl-MaleimidoPropionic Acyl
HXDGTFTSDFEHYLEDAVQKAFIAWLSTGVPSSGAPPPSK

SEQ ID NO: 22 - protein, 40 aa, synthetic construct
  MOD_RES 13: note = Aib
  MOD_RES 20: note = Aib
  MOD_RES 40: note = AminoEthoxyEthoxyAcetic Acyl-MaleimidoPropionic Acyl
FGDGTFTSDLRGXLEIMPQXAFVNWLTSGKPSSGAPPPSK

SEQ ID NO: 23 - protein, 40 aa, synthetic construct
  SITE 2..3: note = D-alanine
  SITE 10: note = D-serine
  MOD_RES 40: note = AminoEthoxyEthoxyAcetic Acyl-MaleimidoPropionic Acyl
HAAGTFTSDSMIYLDNAHQQEFIQWLFNGPPSSGAPPPSK

SEQ ID NO: 24 - protein, 40 aa, synthetic construct
  SITE 2: note = D-alanine
  SITE 10: note = D-serine
  MOD_RES 12: note = Aib
  SITE 16: note = D-alanine
  MOD_RES 20: note = Aib
  MOD_RES 40: note = AminoEthoxyEthoxyAcetic Acyl-MaleimidoPropionic Acyl
HAIGTFTSDSTXYLEAYKQXDFVQWLHSGGPSSGAPPPSK

SEQ ID NO: 25 - protein, 40 aa, synthetic construct
  SITE 2: note = D-serine
  SITE 10: note = D-alanine
  SITE 17: note = D-serine
  MOD_RES 40: note = AminoEthoxyEthoxyAcetic Acyl-MaleimidoPropionic Acyl
  SITE 30: note = D-alanine
YSWGTFTSDAGYYLDMSFQQAFIQWLKAGAPSSGAPPPSK

SEQ ID NO: 26 - protein, 40 aa, synthetic construct
  MOD_RES 13: note = Aib
  SITE 17: note = D-alanine
  SITE 28: note = D-serine
  MOD_RES 40: note = AminoEthoxyEthoxyAcetic Acyl-MaleimidoPropionic Acyl
YGRGTFTSDWIYXLDTAPQKEFVEWLHSGAPSSGAPPPSK

SEQ ID NO: 27 - protein, 40 aa, synthetic construct
  SITE 2: note = D-alanine
  SITE 10: note = D-serine
  MOD_RES 13: note = Aib
  MOD_RES 40: note = AminoEthoxyEthoxyAcetic Acyl-MaleimidoPropionic Acyl
YAMGTFTSDSEEXLEMVYQQAFVAWLPVGGPSSGAPPPSK

SEQ ID NO: 28 - protein, 40 aa, synthetic construct
  SITE 2..3: note = D-serine
  MOD_RES 13: note = Aib
  MOD_RES 17: note = Aib
  MOD_RES 20: note = Aib
  MOD_RES 40: note = AminoEthoxyEthoxyAcetic Acyl-MaleimidoPropionic Acyl
FSSGTFTSDWDWXLDIXIAXEFINWLVYGYPSSGAPPPSK

SEQ ID NO: 29 - protein, 40 aa, synthetic construct
  MOD_RES 2: note = Aib
  MOD_RES 40: note = AminoEthoxyEthoxyAcetic Acyl-MaleimidoPropionic Acyl
FXGGTFTSDDMRYLEPKGQQAFIQWLWVGQPSSGAPPPSK

SEQ ID NO: 30 - protein, 40 aa, synthetic construct
  MOD_RES 2: note = Aib
  MOD_RES 30: note = Aib
  MOD_RES 40: note = AminoEthoxyEthoxyAcetic Acyl-MaleimidoPropionic Acyl
YXSGTFTSDVETYLDLLIAKEFIAWLGAGXPSSGAPPPSK

SEQ ID NO: 31 - protein, 40 aa, synthetic construct
  MOD_RES 13: note = Aib
  MOD_RES 20: note = Aib
  MOD_RES 40: note = AminoEthoxyEthoxyAcetic Acyl-MaleimidoPropionic Acyl
HGRGTFTSDHQFXLDSMIQXEFIQWLHYGIPSSGAPPPSK

SEQ ID NO: 32 - protein, 40 aa, synthetic construct
  SITE 2: note = D-alanine
  MOD_RES 13: note = Aib
  SITE 16: note = D-serine
  SITE 30: note = D-alanine
  MOD_RES 40: note = AminoEthoxyEthoxyAcetic Acyl-MaleimidoPropionic Acyl
YARGTFTSDFINXLESGVAKAFVNWLQEGAPSSGAPPPSK

SEQ ID NO: 33 - protein, 40 aa, synthetic construct
  SITE 2: note = D-serine
  SITE 28: note = D-alanine
  SITE 30: note = D-alanine
  MOD_RES 40: note = AminoEthoxyEthoxyAcetic Acyl-MaleimidoPropionic Acyl
HSFGTFTSDGWMYLDMNEQKAFVAWLYAGAPSSGAPPPSK

SEQ ID NO: 34 - protein, 40 aa, synthetic construct
  SITE 2: note = D-serine
  MOD_RES 13: note = Aib
  MOD_RES 20: note = Aib
  MOD_RES 40: note = AminoEthoxyEthoxyAcetic Acyl-MaleimidoPropionic Acyl
FSRGTFTSDHYRXLDQSKAXEFIEWLESGNPSSGAPPPSK

SEQ ID NO: 35 - protein, 40 aa, synthetic construct
  SITE 2: note = D-serine
  MOD_RES 40: note = Aib
FSGGTFTSDHHRYLDQIPQKDFIQWLEFGKPSSGAPPPSK

SEQ ID NO: 36 - protein, 40 aa, synthetic construct
  SITE 2: note = D-alanine
  SITE 10: note = D-alanine
  MOD_RES 13: note = Aib
  SITE 18: note = D-alanine
  MOD_RES 40: note = AminoEthoxyEthoxyAcetic Acyl-MaleimidoPropionic Acyl
FAQGTFTSDANMXLDMFAQKAFINWLFQGGPSSGAPPPSK

SEQ ID NO: 37 - protein, 40 aa, synthetic construct
  SITE 2: note = D-alanine
  SITE 12: note = D-alanine
  MOD_RES 13: note = Aib
  MOD_RES 17: note = Aib
  MOD_RES 40: note = AminoEthoxyEthoxyAcetic Acyl-MaleimidoPropionic Acyl
FAAGTFTSDVTAXLEVXQQKAFIAWLNLGQPSSGAPPPSK

SEQ ID NO: 38 - protein, 40 aa, synthetic construct
  SITE 2: note = D-alanine
  MOD_RES 12: note = Aib
  SITE 16: note = D-serine
  MOD_RES 40: note = AminoEthoxyEthoxyAcetic Acyl-MaleimidoPropionic Acyl
HAIGTFTSDHMXYLDSDPAQDFIEWLMPGSPSSGAPPPSK

SEQ ID NO: 39 - protein, 40 aa, synthetic construct
  MOD_RES 2: note = Aib
  MOD_RES 40: note = AminoEthoxyEthoxyAcetic Acyl-MaleimidoPropionic Acyl
HXNGTFTSDIIHYLDPIVQKAFIEWLNGGAPSSGAPPPSK

SEQ ID NO: 40 - protein, 40 aa, synthetic construct
  MOD_RES 11: note = Aib
  MOD_RES 13: note = Aib
  MOD_RES 40: note = AminoEthoxyEthoxyAcetic Acyl-MaleimidoPropionic Acyl
HGEGTFTSDMXQXLDREIAKEFVEWLFFGYPSSGAPPPSK

SEQ ID NO: 41 - protein, 40 aa, synthetic construct
  SITE 2: note = D-serine
  SITE 12: note = D-serine
  MOD_RES 13: note = Aib
  SITE 28: note = D-serine
  MOD_RES 40: note = AminoEthoxyEthoxyAcetic Acyl-MaleimidoPropionic Acyl
HSPGTFTSDPDSXLDENHQQAFVAWLNSGMPSSGAPPPSK

SEQ ID NO: 42 - protein, 40 aa, synthetic construct
  SITE 2: note = D-serine
  SITE 3: note = D-alanine
  SITE 10: note = D-serine
  SITE 16..17: note = D-serine
  MOD_RES 20: note = Aib
  MOD_RES 40: note = AminoEthoxyEthoxyAcetic Acyl-MaleimidoPropionic Acyl
FSAGTFTSDSLDYLESSEQXEFINWLAAGVPSSGAPPPSK

SEQ ID NO: 43 - protein, 40 aa, synthetic construct
  SITE 2: note = D-alanine
  MOD_RES 3: note = Aib
  MOD_RES 16: note = Aib
  MOD_RES 20: note = Aib
  SITE 28: note = D-serine
  MOD_RES 40: note = AminoEthoxyEthoxyAcetic Acyl-MaleimidoPropionic Acyl
YAXGTFTSDKDPYLEXTYAXEFVAWLVSGRPSSGAPPPSK

SEQ ID NO: 44 - protein, 40 aa, synthetic construct
  MOD_RES 13: note = Aib
  MOD_RES 40: note = AminoEthoxyEthoxyAcetic Acyl-MaleimidoPropionic Acyl
FGRGTFTSDAVMXLDAMQAKAFIEWLWIGIPSSGAPPPSK

SEQ ID NO: 45 - protein, 40 aa, synthetic construct
  SITE 2..3: note = D-alanine
  MOD_RES 13: note = Aib
  MOD_RES 40: note = AminoEthoxyEthoxyAcetic Acyl-MaleimidoPropionic Acyl
HAAGTFTSDVDYXLEWKIQQDFIEWLSTGLPSSGAPPPSK

SEQ ID NO: 46 - protein, 40 aa, synthetic construct
  SITE 2: note = D-serine
  MOD_RES 18: note = Aib
  MOD_RES 20: note = Aib
  MOD_RES 40: note = AminoEthoxyEthoxyAcetic Acyl-MaleimidoPropionic Acyl
YSFGTFTSDIAGYLDLEXAXAFVQWLAHGKPSSGAPPPSK

SEQ ID NO: 47 - protein, 40 aa, synthetic construct
  SITE 2: note = D-serine
  MOD_RES 40: note = AminoEthoxyEthoxyAcetic Acyl-MaleimidoPropionic Acyl
HSHGTFTSDEVQYLDMDGQQEFINWLDLGYPSSGAPPPSK

SEQ ID NO: 48 - protein, 40 aa, synthetic construct
  SITE 2: note = D-serine
  MOD_RES 13: note = Aib
  MOD_RES 20: note = Aib
  MOD_RES 27: note = Aib
  MOD_RES 40: note = AminoEthoxyEthoxyAcetic Acyl-MaleimidoPropionic Acyl
HSRGTFTSDLQGXLEYADQXDFVNWLXTGFPSSGAPPPSK

SEQ ID NO: 49 - protein, 40 aa, synthetic construct
  MOD_RES 13: note = Aib
  SITE 16: note = D-alanine
  MOD_RES 27: note = D-alanine
  MOD_RES 40: note = AminoEthoxyEthoxyAcetic Acyl-MaleimidoPropionic Acyl
HGFGTFTSDGIDXLEAITAKEFIAWLAIGKPSSGAPPPSK

SEQ ID NO: 50 - protein, 40 aa, synthetic construct
  MOD_RES 2: note = Aib
  SITE 11: note = D-alanine
  MOD_RES 13: note = Aib
  MOD_RES 20: note = Aib
  MOD_RES 40: note = AminoEthoxyEthoxyAcetic Acyl-MaleimidoPropionic Acyl
HXTGTFTSDIAIXLDYKGAXAFVQWLDAGEPSSGAPPPSK

SEQ ID NO: 51 - protein, 40 aa, synthetic construct
  SITE 2: note = D-serine
  MOD_RES 11: note = Aib
  MOD_RES 13: note = Aib
  MOD_RES 20: note = Aib
  MOD_RES 40: note = AminoEthoxyEthoxyAcetic Acyl-MaleimidoPropionic Acyl
HSLGTFTSDLXMXLDVNIAXDFIAWLMIGYPSSGAPPPSK

SEQ ID NO: 52 - protein, 40 aa, synthetic construct
  MOD_RES 13: note = Aib
  SITE 28: note = D-serine
  MOD_RES 40: note = AminoEthoxyEthoxyAcetic Acyl-MaleimidoPropionic Acyl
YGDGTFTSDKHRXLEISQQKEFVQWLLSGLPSSGAPPPSK

SEQ ID NO: 53 - protein, 40 aa, synthetic construct
  SITE 2..3: note = D-alanine
  MOD_RES 13: note = Aib
  MOD_RES 28: note = Aib
  MOD_RES 30: note = Aib
  MOD_RES 40: note = AminoEthoxyEthoxyAcetic Acyl-MaleimidoPropionic Acyl
FAAGTFTSDQTTXLDAGSAKDFVNWLVXGXPSSGAPPPSK

SEQ ID NO: 54 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
YXGGTFTSDEPPYLDAWTQXEFVEWLVRGWPSSGAPPPSK

SEQ ID NO: 55 - protein, 40 aa, synthetic construct
  SITE 2: note = D-alanine
  MOD_RES 13: note = Aib
  SITE 17: note = D-alanine
  MOD_RES 40: note = AminoEthoxyEthoxyAcetic Acyl-MaleimidoPropionic Acyl
HAGGTFTSDGSRXLDIATQQDFIEWLLEGHPSSGAPPPSK

SEQ ID NO: 56 - protein, 40 aa, synthetic construct
  SITE 2: note = D-serine
  MOD_RES 40: note = AminoEthoxyEthoxyAcetic Acyl-MaleimidoPropionic Acyl
HSSGTFTSDPLKYLDHGNQQAFVNWLISGSPSSGAPPPSK

SEQ ID NO: 57 - protein, 40 aa, synthetic construct
  MOD_RES 2: note = Aib
  SITE 30: note = D-serine
  MOD_RES 40: note = AminoEthoxyEthoxyAcetic Acyl-MaleimidoPropionic Acyl
HXTGTFTSDISFYLEEYVAQEFVAWLQAGSPSSGAPPPSK

SEQ ID NO: 58 - protein, 40 aa, synthetic construct
  SITE 2: note = D-serine
  SITE 3: note = D-alanine
  SITE 10: note = D-alanine
  MOD_RES 13: note = Aib
  MOD_RES 17: note = Aib
  MOD_RES 40: note = AminoEthoxyEthoxyAcetic Acyl-MaleimidoPropionic Acyl
HSAGTFTSDAYEXLEPXDQKAFIAWLWHGLPSSGAPPPSK

SEQ ID NO: 59 - protein, 40 aa, synthetic construct
  SITE 2: note = D-serine
  MOD_RES 13: note = Aib
  SITE 17: note = D-serine
  MOD_RES 40: note = AminoEthoxyEthoxyAcetic Acyl-MaleimidoPropionic Acyl
YSRGTFTSDATLXLEGSKQKAFINWLLEGIPSSGAPPPSK

SEQ ID NO: 60 - protein, 40 aa, synthetic construct
  MOD_RES 13: note = Aib
  MOD_RES 40: note = AminoEthoxyEthoxyAcetic Acyl-MaleimidoPropionic Acyl
FGWGTFTSDLEKXLELARAQAFVEWLKVGSPSSGAPPPSK

SEQ ID NO: 61 - protein, 40 aa, synthetic construct
  MOD_RES 2: note = Aib
  SITE 18: note = D-alanine
  SITE 27: note = D-serine
  MOD_RES 40: note = AminoEthoxyEthoxyAcetic Acyl-MaleimidoPropionic Acyl
FXMGTFTSDPYPYLEWEAAQAFINWLSGGSPSSGAPPPSK

SEQ ID NO: 62 - protein, 40 aa, synthetic construct
  SITE 2: note = D-alanine
  MOD_RES 3: note = Aib
  MOD_RES 13: note = Aib
  MOD_RES 40: note = AminoEthoxyEthoxyAcetic Acyl-MaleimidoPropionic Acyl
HAXGTFTSDMPTXLDHIQQQDFVAWLVQGIPSSGAPPPSK

SEQ ID NO: 63 - protein, 40 aa, synthetic construct
  SITE 2: note = D-alanine
  SITE 3: note = D-serine
  MOD_RES 13: note = Aib
  MOD_RES 40: note = AminoEthoxyEthoxyAcetic Acyl-MaleimidoPropionic Acyl
FASGTFTSDHSDXLELKAQQEFVNWLRNGRPSSGAPPPSK

SEQ ID NO: 64 - protein, 40 aa, synthetic construct
  MOD_RES 40: note = AminoEthoxyEthoxyAcetic Acyl-MaleimidoPropionic Acyl
HGQGTFTSDYSKYLDARRAQDFVEWLKNGGPSSGAPPPSK

SEQ ID NO: 65 - protein, 40 aa, synthetic construct
  MOD_RES 2: note = Aib
  MOD_RES 13: note = Aib
  MOD_RES 40: note = AminoEthoxyEthoxyAcetic Acyl-MaleimidoPropionic Acyl
HXQGTFTSDYSKXLDKRRAKDFVEWLKNGGPSSGAPPPSK

SEQ ID NO: 66 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDKRRAKDFVEWLKNGGPSSGAPPPSK

SEQ ID NO: 67 - protein, 40 aa, synthetic construct
  MOD_RES 2: note = Aib
  MOD_RES 13: note = Alpha-Methyl-Leucine
  MOD_RES 20: note = Aib
  MOD_RES 40: note = AminoEthoxyEthoxyAcetic Acyl-MaleimidoPropionic Acyl
YXQGTFTSDYSIXLDKIAQXAFIEYLLEGGPSSGAPPPSK

SEQ ID NO: 68 - protein, 40 aa, synthetic construct
  MOD_RES 2: note = Aib
  MOD_RES 13: note = Aib
  MOD_RES 40: note = AminoEthoxyEthoxyAcetic Acyl-MaleimidoPropionic Acyl
YXEGTFTSDYSIXLDKIAQKAFVQWLIAGGPSSGAPPPSK

SEQ ID NO: 69 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFIAWLVNGGPSSGAPPPSK

SEQ ID NO: 70 - protein, 40 aa, synthetic construct; identical to SEQ ID NO: 14
  MOD_RES 2: note = Aib
  MOD_RES 20: note = Aib
  MOD_RES 40: note = AminoEthoxyEthoxyAcetic Acyl-MaleimidoPropionic Acyl

SEQ ID NO: 71 - protein, 40 aa, synthetic construct
  MOD_RES 2: note = Aib
  MOD_RES 20: note = Aib
  MOD_RES 35: note = Aib
  MOD_RES 40: note = AminoEthoxyEthoxyAcetic Acyl-MaleimidoPropionic Acyl
  SITE 29: note = D-alanine
HXHGTFTSDLSKLLEEQRQXEFIEWLKAAGPPPSXKPPPK

SEQ ID NO: 72 - protein, 39 aa, synthetic construct
  SITE 2: note = D-alanine
  MOD_RES 20: note = AminoEthoxyEthoxyAcetic Acyl-MaleimidoPropionic Acyl
HAMGTFTSDRHWYLDMSHQKAFVQWLAYGNPSSGAPPPS

SEQ ID NO: 73 - protein, 39 aa, synthetic construct
  SITE 2: note = D-alanine
  SITE 3: note = D-serine
  MOD_RES 20: note = AminoEthoxyEthoxyAcetic Acyl-MaleimidoPropionic Acyl
YASGTFTSDWGRYLELLIQKEFVNWLIIGAPSSGAPPPS

SEQ ID NO: 74 - protein, 39 aa, synthetic construct
  SITE 10: note = D-serine
  MOD_RES 13: note = Aib
  SITE 17: note = D-serine
  SITE 18: note = D-alanine
  SITE 27: note = D-alanine
  MOD_RES 20: note = AminoEthoxyEthoxyAcetic Acyl-AminoEthoxyEthoxyAcetic Acyl-MaleimidoPropionic Acyl
HGKGTFTSDSAIXLEVSAAKAFIEWLAHGDPSSGAPPPS

SEQ ID NO: 75 - protein, 39 aa, synthetic construct
  SITE 3: note = D-serine
  SITE 2: note = D-alanine
  MOD_RES 13: note = Aib
  SITE 27: note = D-alanine
  MOD_RES 20: note = AminoEthoxyEthoxyAcetic Acyl-MaleimidoPropionic Acyl
HASGTFTSDILEXLDQAAAKEFVEWLAHGFPSSGAPPPS

SEQ ID NO: 76 - protein, 39 aa, synthetic construct
  SITE 2: note = D-alanine
  MOD_RES 12..13: note = Aib
  MOD_RES 20: note = AminoEthoxyEthoxyAcetic Acyl-MaleimidoPropionic Acyl
HAYGTFTSDYMXXLDFLQQKDFVAWLFMGVPSSGAPPPS

SEQ ID NO: 77 - protein, 39 aa, synthetic construct
  MOD_RES 2: note = Aib
  SITE 3: note = D-serine
  MOD_RES 10: note = Aib
  MOD_RES 20: note = AminoEthoxyEthoxyAcetic Acyl-MaleimidoPropionic Acyl
HXSGTFTSDXAYYLDNTTAKDFIQWLDAGPPSSGAPPPS

SEQ ID NO: 78 - protein, 39 aa, synthetic construct
  MOD_RES 2: note = Aib
  SITE 11: note = D-alanine
  MOD_RES 16: note = Aib
  SITE 30: note = D-serine
  MOD_RES 20: note = AminoEthoxyEthoxyAcetic Acyl-MaleimidoPropionic Acyl
FXKGTFTSDDAVYLEXHRAKEFVEWLFGGSPSSGAPPPS

SEQ ID NO: 79 - protein, 39 aa, synthetic construct
  MOD_RES 2: note = Aib
  MOD_RES 13: note = Aib
  MOD_RES 27: note = Aib
  MOD_RES 20: note = AminoEthoxyEthoxyAcetic Acyl-MaleimidoPropionic Acyl
YXHGTFTSDLTVXLDEIKAKEFVEWLXVGPPSSGAPPPS

SEQ ID NO: 80 - protein, 39 aa, synthetic construct
  SITE 2: note = D-serine
  SITE 12: note = D-alanine
  MOD_RES 13: note = Aib
  MOD_RES 16: note = Aib
  MOD_RES 20: note = AminoEthoxyEthoxyAcetic Acyl-MaleimidoPropionic Acyl
FSWGTFTSDFPAXLEXMKAKDFVEWLLDGNPSSGAPPPS

SEQ ID NO: 81 - protein, 39 aa, synthetic construct
  MOD_RES 2: note = Aib
  MOD_RES 13: note = Aib
  SITE 28: note = D-serine
  MOD_RES 20: note = AminoEthoxyEthoxyAcetic Acyl-MaleimidoPropionic Acyl
HXKGTFTSDDADXLEWYRQKAFVQWLPSGIPSSGAPPPS

SEQ ID NO: 82 - protein, 39 aa, synthetic construct
  MOD_RES 2: note = Aib
  MOD_RES 13: note = Aib
  SITE 27: note = D-serine
  MOD_RES 20: note = AminoEthoxyEthoxyAcetic Acyl-MaleimidoPropionic Acyl
YXWGTFTSDSNSXLEAKMQKEFVNWLSQGFPSSGAPPPS

SEQ ID NO: 83 - protein, 39 aa, synthetic construct
  SITE 2: note = D-alanine
  SITE 18: note = D-serine
  MOD_RES 20: note = AminoEthoxyEthoxyAcetic Acyl-MaleimidoPropionic Acyl
FATGTFTSDDADYLELFSQKAFIAWLDNGVPSSGAPPPS

SEQ ID NO: 84 - protein, 39 aa, synthetic construct
  SITE 2: note = D-serine
  MOD_RES 13: note = Aib
  MOD_RES 17: note = Aib
  MOD_RES 20: note = AminoEthoxyEthoxyAcetic Acyl-MaleimidoPropionic Acyl
YSYGTFTSDWDAXLESXMQKEFVQWLFYGQPSSGAPPPS

SEQ ID NO: 85 - protein, 39 aa, synthetic construct
  SITE 2: note = D-alanine
  SITE 12: note = D-serine
  MOD_RES 13: note = Aib
  MOD_RES 20: note = AminoEthoxyEthoxyAcetic Acyl-MaleimidoPropionic Acyl
YANGTFTSDTNSXLDSTQAKEFVAWLVQGDPSSGAPPPS

SEQ ID NO: 86 - protein, 39 aa, synthetic construct
  MOD_RES 2: note = Aib
  MOD_RES 20: note = AminoEthoxyEthoxyAcetic Acyl-MaleimidoPropionic Acyl
FXIGTFTSDKQAYLEHPRQKAFVAWLDVGYPSSGAPPPS

SEQ ID NO: 87 - protein, 39 aa, synthetic construct
  MOD_RES 2: note = Aib
  MOD_RES 13: note = Aib
  SITE 30: note = D-serine
  MOD_RES 20: note = AminoEthoxyEthoxyAcetic Acyl-MaleimidoPropionic Acyl
HXRGTFTSDKFIXLEYHNAKEFVAWLYKGSPSSGAPPPS

SEQ ID NO: 88 - protein, 39 aa, synthetic construct
  SITE 10: note = D-serine
  MOD_RES 20: note = AminoEthoxyEthoxyAcetic Acyl-MaleimidoPropionic Acyl
FGHGTFTSDSIWYLENYSQKEFIEWLEKGPPSSGAPPPS

SEQ ID NO: 89 - protein, 39 aa, synthetic construct
  SITE 2: note = D-serine
  SITE 3: note = D-alanine
  MOD_RES 12: note = Aib
  SITE 27: note = D-alanine
  MOD_RES 20: note = AminoEthoxyEthoxyAcetic Acyl-MaleimidoPropionic Acyl
HSAGTFTSDIRXYLEIMLQKEFVEWLAEGVPSSGAPPPS

SEQ ID NO: 90 - protein, 39 aa, synthetic construct
  MOD_RES 2: note = Aib
  SITE 18: note = D-serine
  MOD_RES 20: note = AminoEthoxyEthoxyAcetic Acyl-MaleimidoPropionic Acyl
HXFGTFTSDMLNYLEENSQKEFVNWLQLGMPSSGAPPPS

SEQ ID NO: 91 - protein, 39 aa, synthetic construct
  MOD_RES 20: note = AminoEthoxyEthoxyAcetic Acyl-MaleimidoPropionic Acyl
FGHGTFTSDIWIYLEVQTAKDFINWLSWGEPSSGAPPPS

SEQ ID NO: 92 - protein, 39 aa, synthetic construct
  SITE 2: note = D-serine
  MOD_RES 20: note = AminoEthoxyEthoxyAcetic Acyl-MaleimidoPropionic Acyl
HSGGTFTSDSGPYLDKTDQKAFINWLPIGNPSSGAPPPS

SEQ ID NO: 93 - protein, 39 aa, synthetic construct
  SITE 2: note = D-serine
  SITE 11: note = D-serine
  MOD_RES 13: note = Aib
  MOD_RES 20: note = AminoEthoxyEthoxyAcetic Acyl-MaleimidoPropionic Acyl
YSIGTFTSDHSPXLDHLFAKDFVEWLENGDPSSGAPPPS

SEQ ID NO: 94 - protein, 39 aa, synthetic construct
  MOD_RES 13: note = Aib
  MOD_RES 18: note = Aib
  SITE 17: note = D-serine
  MOD_RES 20: note = AminoEthoxyEthoxyAcetic Acyl-MaleimidoPropionic Acyl
FGYGTFTSDKEGXLEQSXAKEFIQWLPHGPPSSGAPPPS

SEQ ID NO: 95 - protein, 39 aa, synthetic construct
  SITE 2: note = D-serine
  MOD_RES 11: note = Aib
  MOD_RES 13: note = Aib
  MOD_RES 20: note = AminoEthoxyEthoxyAcetic Acyl-MaleimidoPropionic Acyl
FSWGTFTSDFXSXLDTTRAKDFVEWLIRGNPSSGAPPPS

SEQ ID NO: 96 - protein, 39 aa, synthetic construct
  MOD_RES 2: note = Aib
  SITE 18: note = D-serine
  MOD_RES 28: note = Aib
  MOD_RES 20: note = AminoEthoxyEthoxyAcetic Acyl-MaleimidoPropionic Acyl
HXPGTFTSDHEKYLEMVSAKDFIAWLRXGDPSSGAPPPS

SEQ ID NO: 97 - protein, 39 aa, synthetic construct
  SITE 2: note = D-alanine
  SITE 12: note = D-alanine
  MOD_RES 13: note = Aib
  SITE 17: note = D-alanine
  MOD_RES 20: note = AminoEthoxyEthoxyAcetic Acyl-MaleimidoPropionic Acyl
YAAGTFTSDTIAXLDNAAAKDFIAWLIQGYPSSGAPPPS

SEQ ID NO: 98 - protein, 39 aa, synthetic construct
  MOD_RES 2: note = Aib
  MOD_RES 13: note = Aib
  MOD_RES 20: note = AminoEthoxyEthoxyAcetic Acyl-MaleimidoPropionic Acyl
YXLGTFTSDTIHXLEFEAQKDFINWLKAGEPSSGAPPPS

SEQ ID NO: 99 - protein, 39 aa, synthetic construct
  MOD_RES 2: note = Aib
  MOD_RES 27: note = Aib
  MOD_RES 20: note = AminoEthoxyEthoxyAcetic Acyl-MaleimidoPropionic Acyl
YXQGTFTSDAMIYLDTPDAKEFIAWLXIGGPSSGAPPPS

SEQ ID NO: 100 - protein, 39 aa, synthetic construct
  MOD_RES 11: note = Aib
  MOD_RES 13: note = Aib
  MOD_RES 17: note = Aib
  MOD_RES 20: note = AminoEthoxyEthoxyAcetic Acyl-MaleimidoPropionic Acyl
YGAGTFTSDHXGXLDIXQQKEFVQWLETGTPSSGAPPPS

SEQ ID NO: 101 - protein, 39 aa, synthetic construct
  SITE 2: note = D-alanine
  MOD_RES 20: note = AminoEthoxyEthoxyAcetic Acyl-MaleimidoPropionic Acyl
YAVGTFTSDLSEYLEMNIAKDFVQWLLVGGPSSGAPPPS

SEQ ID NO: 102 - protein, 39 aa, synthetic construct
  SITE 12: note = D-serine
  MOD_RES 20: note = AminoEthoxyEthoxyAcetic Acyl-MaleimidoPropionic Acyl
YGPGTFTSDRYSYLEQHMAKEFIQWLGPGHPSSGAPPPS

SEQ ID NO: 103 - protein, 39 aa, synthetic construct
  MOD_RES 2: note = Aib
  SITE 28: note = D-alanine
  MOD_RES 20: note = AminoEthoxyEthoxyAcetic Acyl-MaleimidoPropionic Acyl
YXRGTFTSDMRVYLEETLQKAFVQWLIAGDPSSGAPPPS

SEQ ID NO: 104 - protein, 39 aa, synthetic construct
  SITE 2: note = D-serine
  MOD_RES 3: note = Aib
  MOD_RES 13: note = Aib
  MOD_RES 20: note = AminoEthoxyEthoxyAcetic Acyl-MaleimidoPropionic Acyl
YSXGTFTSDFMEXLDKKIQKDFIQWLWYGSPSSGAPPPS

SEQ ID NO: 105 - protein, 39 aa, synthetic construct
  MOD_RES 2: note = Aib
  SITE 3: note = D-serine
  MOD_RES 27: note = Aib
  MOD_RES 20: note = AminoEthoxyEthoxyAcetic Acyl-MaleimidoPropionic Acyl
FXSGTFTSDELRYLEEMHQKAFIQWLXTGFPSSGAPPPS

SEQ ID NO: 106 - protein, 39 aa, synthetic construct
  SITE 27: note = D-serine
  MOD_RES 20: note = AminoEthoxyEthoxyAcetic Acyl-MaleimidoPropionic Acyl
YGPGTFTSDWAKYLEGRDAKAFINWLSQGSPSSGAPPPS

SEQ ID NO: 107 - protein, 39 aa, synthetic construct
  MOD_RES 13: note = Aib
  SITE 30: note = D-alanine
  MOD_RES 20: note = AminoEthoxyEthoxyAcetic Acyl-MaleimidoPropionic Acyl
HGVGTFTSDHHFXLEIIMAKAFIEWLWHGAPSSGAPPPS

SEQ ID NO: 108 - protein, 39 aa, synthetic construct
  SITE 18: note = D-alanine
  MOD_RES 20: note = AminoEthoxyEthoxyAcetic Acyl-MaleimidoPropionic Acyl
HGIGTFTSDVPAYLDPAAAKDFIQWLVSGGPSSGAPPPS

SEQ ID NO: 109 - protein, 39 aa, synthetic construct
  SITE 2: note = D-serine
  MOD_RES 13: note = Aib
  MOD_RES 28: note = Aib
  MOD_RES 20: note = AminoEthoxyEthoxyAcetic Acyl-MaleimidoPropionic Acyl
HSNGTFTSDRRKXLEPPIAKEFVQWLIXGIPSSGAPPPS

SEQ ID NO: 110 - protein, 39 aa, synthetic construct
  MOD_RES 2: note = Aib
  SITE 12: note = D-alanine
  MOD_RES 20: note = AminoEthoxyEthoxyAcetic Acyl-MaleimidoPropionic Acyl
FXTGTFTSDTMAYLDYTHAKDFIAWLIDGKPSSGAPPPS

SEQ ID NO: 111 - protein, 39 aa, synthetic construct
  SITE 2: note = D-alanine
  SITE 10: note = D-serine
  MOD_RES 20: note = AminoEthoxyEthoxyAcetic Acyl-MaleimidoPropionic Acyl
YATGTFTSDSVQYLEHPMQKAFVAWLHTGRPSSGAPPPS

SEQ ID NO: 112 - protein, 39 aa, synthetic construct
  MOD_RES 2: note = Aib
  MOD_RES 30: note = Aib
  MOD_RES 20: note = AminoEthoxyEthoxyAcetic Acyl-MaleimidoPropionic Acyl
YXTGTFTSDEEWYLDNWMQKAFVQWLSNGXPSSGAPPPS

SEQ ID NO: 113 - protein, 39 aa, synthetic construct
  SITE 2: note = D-serine
  MOD_RES 13: note = Aib
  MOD_RES 16: note = Aib
  MOD_RES 20: note = AminoEthoxyEthoxyAcetic Acyl-MaleimidoPropionic Acyl
YSIGTFTSDGNKXLEXGRAKAFIAWLQYGRPSSGAPPPS

SEQ ID NO: 114 - protein, 39 aa, synthetic construct
  SITE 2: note = D-serine
  MOD_RES 20: note = AminoEthoxyEthoxyAcetic Acyl-MaleimidoPropionic Acyl
YSPGTFTSDMYVYLEPGDAKEFVQWLKNGGPSSGAPPPS

SEQ ID NO: 115 - protein, 39 aa, synthetic construct
  SITE 2: note = D-alanine
  MOD_RES 13: note = Aib
  SITE 30: note = D-serine
  MOD_RES 20: note = AminoEthoxyEthoxyAcetic Acyl-MaleimidoPropionic Acyl
FSIGTFTSDLMAXLERAAQKEFVNWLIIGSPSSGAPPPS

SEQ ID NO: 116 - protein, 39 aa, synthetic construct
  MOD_RES 2: note = Aib
  MOD_RES 20: note = AminoEthoxyEthoxyAcetic Acyl-MaleimidoPropionic Acyl
YXNGTFTSDIEQYLEPMVQKEFVQWLSPGNPSSGAPPPS

SEQ ID NO: 117 - protein, 39 aa, synthetic construct
  SITE 12: note = D-serine
  MOD_RES 13: note = Aib
  MOD_RES 20: note = AminoEthoxyEthoxyAcetic Acyl-MaleimidoPropionic Acyl
YGWGTFTSDSFSXLDWMMAKAFIQWLIVGIPSSGAPPPS

SEQ ID NO: 118 - protein, 39 aa, synthetic construct
  SITE 2: note = D-serine
  MOD_RES 10: note = Aib
  MOD_RES 20: note = AminoEthoxyEthoxyAcetic Acyl-MaleimidoPropionic Acyl
HSPGTFTSDXTHYLDNDQQKDFVNWLPEGWPSSGAPPPS

SEQ ID NO: 119 - protein, 39 aa, synthetic construct
  SITE 3: note = D-alanine
  MOD_RES 13: note = Aib
  SITE 16: note = D-serine
  MOD_RES 20: note = AminoEthoxyEthoxyAcetic Acyl-MaleimidoPropionic Acyl
YGAGTFTSDNHTXLESFAAKEFIQWLNSGQPSSGAPPPS

SEQ ID NO: 120 - protein, 39 aa, synthetic construct
  SITE 2: note = D-alanine
  MOD_RES 13: note = Aib
  MOD_RES 20: note = AminoEthoxyEthoxyAcetic Acyl-MaleimidoPropionic Acyl
YAMGTFTSDRLRXLDQFSAKAFVNWLSWGEPSSGAPPPS

SEQ ID NO: 121 - protein, 39 aa, synthetic construct
  MOD_RES 2: note = Aib
  MOD_RES 12..13: note = Aib
  SITE 3: note = D-alanine
  SITE 27: note = D-alanine
  MOD_RES 20: note = AminoEthoxyEthoxyAcetic Acyl-MaleimidoPropionic Acyl
FXAGTFTSDSLXXLDHHNQKAFVEWLAPGLPSSGAPPPS

SEQ ID NO: 122 - protein, 39 aa, synthetic construct
  MOD_RES 16: note = AminoEthoxyEthoxyAcetic Acyl-MaleimidoPropionic Acyl
HGQGTFTSDYSKYLDKRRAQDFVEWLKNGGPSSGAPPPS

SEQ ID NO: 123 - protein, 39 aa, synthetic construct
  MOD_RES 2: note = Aib
  MOD_RES 13: note = Aib
  MOD_RES 20: note = AminoEthoxyEthoxyAcetic Acyl-MaleimidoPropionic Acyl
HXQGTFTSDYSKXLDKRRAKDFVEWLKNGGPSSGAPPPS

SEQ ID NO: 124 - protein, 39 aa, synthetic construct
  MOD_RES 2: note = Aib
  MOD_RES 16: note = AminoEthoxyEthoxyAcetic Acyl-MaleimidoPropionic Acyl
HXQGTFTSDYSKYLDKRRAQDFVEWLKNGGPSSGAPPPS

SEQ ID NO: 125 - protein, 39 aa, synthetic construct
  MOD_RES 2: note = Aib
  MOD_RES 13: note = Aib
  MOD_RES 20: note = AminoEthoxyEthoxyAcetic Acyl-MaleimidoPropionic Acyl
YXEGTFTSDYSIXLDKIAQKAFVQWLIAGGPSSGAPPPS

SEQ ID NO: 126 - protein, 39 aa, synthetic construct
  MOD_RES 2: note = Aib
  MOD_RES 13: note = Alpha-Methyl-Leucine
  MOD_RES 20: note = AminoEthoxyEthoxyAcetic Acyl-MaleimidoPropionic Acyl
YXQGTFTSDYSIXLDKIAQKAFIEYLLEGGPSSGAPPPS

SEQ ID NO: 127 - protein, 39 aa, synthetic construct
  SITE 2: note = D-alanine
  MOD_RES 20: note = AminoEthoxyEthoxyAcetic Acyl-MaleimidoPropionic Acyl
HAEGTFTSDYAKYLDARRAKEFIAWLVNGGPSSGAPPPS

SEQ ID NO: 128 - protein, 39 aa, synthetic construct
  SITE 2: note = D-alanine
  SITE 10: note = D-serine
  MOD_RES 20: note = Aib
  MOD_RES 17: note = AminoEthoxyEthoxyAcetic Acyl-MaleimidoPropionic Acyl
YAWGTFTSDSKDYLEFKWAXEFIQWLDPGDPSSGAPPPS

SEQ ID NO: 129 - protein, 39 aa, synthetic construct
  MOD_RES 2: note = Aib
  MOD_RES 13: note = Aib
  MOD_RES 17: note = AminoEthoxyEthoxyAcetic Acyl-MaleimidoPropionic Acyl
  MOD_RES 20: note = Aib
FXYGTFTSDINVXLDIKWAXAFINWLPGGIPSSGAPPPS

SEQ ID NO: 130 - protein, 39 aa, synthetic construct
  SITE 2..3: note = D-alanine
  MOD_RES 13: note = Aib
  MOD_RES 20: note = Aib
  MOD_RES 17: note = AminoEthoxyEthoxyAcetic Acyl-MaleimidoPropionic Acyl
FAAGTFTSDRQEXLDVKLAXEFVQWLVSGDPSSGAPPPS

SEQ ID NO: 131 - protein, 39 aa, synthetic construct
  SITE 2: note = D-alanine
  MOD_RES 13: note = Aib
  SITE 27: note = D-serine
  MOD_RES 17: note = AminoEthoxyEthoxyAcetic Acyl-MaleimidoPropionic Acyl
FARGTFTSDSMQXLETKRQQEFVNWLSMGVPSSGAPPPS

SEQ ID NO: 132 - protein, 39 aa, synthetic construct
  SITE 2: note = D-alanine
  MOD_RES 13: note = Aib
  MOD_RES 20: note = Aib
  MOD_RES 17: note = AminoEthoxyEthoxyAcetic Acyl-MaleimidoPropionic Acyl
YAHGTFTSDTNRXLESKAAXEFIAWLEVGAPSSGAPPPS

SEQ ID NO: 133 - protein, 39 aa, synthetic construct
  MOD_RES 2: note = Aib
  SITE 12: note = D-serine
  MOD_RES 13: note = Aib
  MOD_RES 20: note = Aib
  SITE 16: note = D-serine
  MOD_RES 17: note = AminoEthoxyEthoxyAcetic Acyl-MaleimidoPropionic Acyl
FXGGTFTSDFRSXLDSKIAXEFINWLFKGFPSSGAPPPS

SEQ ID NO: 134 - protein, 39 aa, synthetic construct
  MOD_RES 2: note = Aib
  MOD_RES 17: note = AminoEthoxyEthoxyAcetic Acyl-MaleimidoPropionic Acyl
YXNGTFTSDMMPYLEPKHQKDFVEWLTSGDPSSGAPPPS

SEQ ID NO: 135 - protein, 39 aa, synthetic construct
  SITE 2: note = D-alanine
  MOD_RES 20: note = Aib
  MOD_RES 17: note = AminoEthoxyEthoxyAcetic Acyl-MaleimidoPropionic Acyl
HAPGTFTSDIIHYLETKIAXEFIQWLKRGSPSSGAPPPS

SEQ ID NO: 136 - protein, 39 aa, synthetic construct
  SITE 2: note = D-serine
  MOD_RES 10: note = Aib
  MOD_RES 13: note = Aib
  MOD_RES 20: note = Aib
  SITE 28: note = D-alanine
  MOD_RES 17: note = AminoEthoxyEthoxyAcetic Acyl-MaleimidoPropionic Acyl
FSVGTFTSDXSAXLDLKIAXEFINWLFAGFPSSGAPPPS

SEQ ID NO: 137 - protein, 39 aa, synthetic construct
  MOD_RES 2: note = Aib
  SITE 3: note = D-alanine
  SITE 27: note = D-serine
  MOD_RES 17: note = AminoEthoxyEthoxyAcetic Acyl-MaleimidoPropionic Acyl
YXAGTFTSDGNMYLDKKHAKDFIEWLSSGPPSSGAPPPS

SEQ ID NO: 138 - protein, 39 aa, synthetic construct
  SITE 2: note = D-alanine
  SITE 11: note = D-serine
  MOD_RES 13: note = Aib
  MOD_RES 17: note = AminoEthoxyEthoxyAcetic Acyl-MaleimidoPropionic Acyl
YAIGTFTSDPSHXLEVKNQKEFVQWLNRGIPSSGAPPPS

SEQ ID NO: 139 - protein, 39 aa, synthetic construct
  SITE 2: note = D-serine
  MOD_RES 13: note = Aib
  MOD_RES 17: note = AminoEthoxyEthoxyAcetic Acyl-MaleimidoPropionic Acyl
FSTGTFTSDFVEXLEIKEQKAFIEWLAQGGPSSGAPPPS

SEQ ID NO: 140 - protein, 39 aa, synthetic construct
  SITE 18: note = D-alanine
  SITE 27: note = D-serine
  MOD_RES 17: note = AminoEthoxyEthoxyAcetic Acyl-MaleimidoPropionic Acyl
FGTGTFTSDFDGYLEDKAAKAFIQWLSGGMPSSGAPPPS

SEQ ID NO: 141 - protein, 39 aa, synthetic construct
  SITE 2: note = D-serine
  MOD_RES 13: note = Aib
  MOD_RES 20: note = Aib
  MOD_RES 17: note = AminoEthoxyEthoxyAcetic Acyl-MaleimidoPropionic Acyl
HSWGTFTSDWYIXLEEKNAXDFIAWLYAGYPSSGAPPPS

SEQ ID NO: 142 - protein, 39 aa, synthetic construct
  MOD_RES 13: note = Aib
  MOD_RES 27..28: note = Aib
  MOD_RES 17: note = AminoEthoxyEthoxyAcetic Acyl-MaleimidoPropionic Acyl
FGPGTFTSDKKFXLELKGAKAFIAWLXXGFPSSGAPPPS

SEQ ID NO: 143 - protein, 39 aa, synthetic construct
  SITE 2: note = D-serine
  SITE 10: note = D-alanine
  SITE 12: note = D-serine
  MOD_RES 20: note = Aib
  MOD_RES 28: note = Aib
  MOD_RES 17: note = AminoEthoxyEthoxyAcetic Acyl-MaleimidoPropionic Acyl
HSNGTFTSDANSYLEPKAAXDFVNWLDXGWPSSGAPPPS

SEQ ID NO: 144 - protein, 39 aa, synthetic construct
  SITE 2: note = D-serine
  MOD_RES 17: note = AminoEthoxyEthoxyAcetic Acyl-MaleimidoPropionic Acyl
FSTGTFTSDPWVYLDSKQQQAFVEWLHFGNPSSGAPPPS

SEQ ID NO: 145 - protein, 39 aa, synthetic construct
  SITE 2: note = D-alanine
  MOD_RES 13: note = Aib
  MOD_RES 20: note = Aib
  MOD_RES 17: note = AminoEthoxyEthoxyAcetic Acyl-MaleimidoPropionic Acyl
FAEGTFTSDWYPXLDLKSAXDFVQWLYGGPPSSGAPPPS

SEQ ID NO: 146 - protein, 39 aa, synthetic construct
  MOD_RES 13: note = Aib
  MOD_RES 17: note = AminoEthoxyEthoxyAcetic Acyl-MaleimidoPropionic Acyl
FGIGTFTSDTARXLDDKDQKEFVQWLNDGMPSSGAPPPS

SEQ ID NO: 147 - protein, 39 aa, synthetic construct
  MOD_RES 20: note = Aib
  SITE 30: note = D-serine
  MOD_RES 17: note = AminoEthoxyEthoxyAcetic Acyl-MaleimidoPropionic Acyl
FGYGTFTSDKSTYLDEKIQXDFVEWLNDGSPSSGAPPPS

SEQ ID NO: 148 - protein, 39 aa, synthetic construct
  MOD_RES 11: note = Aib
  MOD_RES 17: note = AminoEthoxyEthoxyAcetic Acyl-MaleimidoPropionic Acyl
HGSGTFTSDAXPYLDQKGAQDFIAWLDGGPPSSGAPPPS

SEQ ID NO: 149 - protein, 39 aa, synthetic construct
  SITE 2: note = D-alanine
  MOD_RES 13: note = Aib
  MOD_RES 17: note = AminoEthoxyEthoxyAcetic Acyl-MaleimidoPropionic Acyl
HAIGTFTSDPPVXLEQKHQQEFVAWLDPGLPSSGAPPPS

SEQ ID NO: 150 - protein, 39 aa, synthetic construct
  SITE 2: note = D-alanine
  MOD_RES 20: note = Aib
  SITE 27: note = D-alanine
  MOD_RES 17: note = AminoEthoxyEthoxyAcetic Acyl-MaleimidoPropionic Acyl
YAYGTFTSDVNKYLDPKTAXAFVEWLAQGIPSSGAPPPS

SEQ ID NO: 151 - protein, 39 aa, synthetic construct
  SITE 2: note = D-alanine
  MOD_RES 20: note = Aib
  MOD_RES 17: note = AminoEthoxyEthoxyAcetic Acyl-MaleimidoPropionic Acyl
FAIGTFTSDRREYLEPKEQXDFIEWLRDGGPSSGAPPPS

SEQ ID NO: 152 - protein, 39 aa, synthetic construct
  MOD_RES 17: note = AminoEthoxyEthoxyAcetic Acyl-MaleimidoPropionic Acyl
YGNGTFTSDAWIYLDDKLQKEFVQWLRLGNPSSGAPPPS

SEQ ID NO: 153 - protein, 39 aa, synthetic construct
  SITE 2: note = D-alanine
  SITE 10..11: note = D-alanine
  MOD_RES 13: note = Aib
  MOD_RES 20: note = Aib
  MOD_RES 27: note = Aib
  MOD_RES 17: note = AminoEthoxyEthoxyAcetic Acyl-MaleimidoPropionic Acyl
YAPGTFTSDAAAXLEDKYQXAFIAWLXQGWPSSGAPPPS

SEQ ID NO: 154 - protein, 39 aa, synthetic construct
  SITE 3: note = D-alanine
  SITE 28: note = D-serine
  MOD_RES 17: note = AminoEthoxyEthoxyAcetic Acyl-MaleimidoPropionic Acyl
YGAGTFTSDIRAYLDEKWAKAFVAWLVSGMPSSGAPPPS

SEQ ID NO: 155 - protein, 39 aa, synthetic construct
  MOD_RES 13: note = Aib
  MOD_RES 18: note = Aib
  MOD_RES 17: note = AminoEthoxyEthoxyAcetic Acyl-MaleimidoPropionic Acyl
YGWGTFTSDPINXLDLKXQKDFVNWLPMGAPSSGAPPPS

SEQ ID NO: 156 - protein, 39 aa, synthetic construct
  SITE 2: note = D-serine
  SITE 10: note = D-serine
  SITE 12: note = D-alanine
  MOD_RES 17: note = AminoEthoxyEthoxyAcetic Acyl-MaleimidoPropionic Acyl
HSNGTFTSDSYAYLERKDQKAFIEWLWSGPPSSGAPPPS

SEQ ID NO: 157 - protein, 39 aa, synthetic construct
  SITE 2: note = D-serine
  MOD_RES 28: note = Aib
  MOD_RES 17: note = AminoEthoxyEthoxyAcetic Acyl-MaleimidoPropionic Acyl
YSMGTFTSDQGKYLEAKSAQAFINWLMXGAPSSGAPPPS

SEQ ID NO: 158 - protein, 39 aa, synthetic construct
  SITE 2: note = D-alanine
  MOD_RES 13: note = Aib
  MOD_RES 16: note = Aib
  SITE 28: note = D-alanine
  MOD_RES 17: note = AminoEthoxyEthoxyAcetic Acyl-MaleimidoPropionic Acyl
YAFGTFTSDLTRXLDXKTAKAFIEWLDAGMPSSGAPPPS

SEQ ID NO: 159 - protein, 39 aa, synthetic construct
  MOD_RES 2: note = Aib
  MOD_RES 10: note = Aib
  MOD_RES 20: note = Aib
  MOD_RES 17: note = AminoEthoxyEthoxyAcetic Acyl-MaleimidoPropionic Acyl
YXKGTFTSDXIYYLDWKNAXAFVNWLHIGIPSSGAPPPS

SEQ ID NO: 160 - protein, 39 aa, synthetic construct
  MOD_RES 2: note = Aib
  SITE 11: note = D-serine
  MOD_RES 12..13: note = Aib
  MOD_RES 20: note = Aib
  MOD_RES 27: note = Aib
  MOD_RES 17: note = AminoEthoxyEthoxyAcetic Acyl-MaleimidoPropionic Acyl
HXQGTFTSDWSXXLEKKVQXEFVNWLXTGDPSSGAPPPS

SEQ ID NO: 161 - protein, 39 aa, synthetic construct
  SITE 2..3: note = D-serine
  SITE 12: note = D-alanine
  MOD_RES 13: note = Aib
  SITE 18: note = D-alanine
  MOD_RES 27: note = Aib
  MOD_RES 17: note = AminoEthoxyEthoxyAcetic Acyl-MaleimidoPropionic Acyl
FSSGTFTSDDRAXLDSKAAQAFVAWLXAGTPSSGAPPPS

SEQ ID NO: 162 - protein, 39 aa, synthetic construct
  SITE 2: note = D-alanine
  SITE 18: note = D-alanine
  SITE 30: note = D-serine
  MOD_RES 17: note = AminoEthoxyEthoxyAcetic Acyl-MaleimidoPropionic Acyl
YAIGTFTSDMKIYLDEKAAKAFVQWLANGSPSSGAPPPS

SEQ ID NO: 163 - protein, 39 aa, synthetic construct
  MOD_RES 13: note = Aib
  MOD_RES 20: note = Aib
  MOD_RES 17: note = AminoEthoxyEthoxyAcetic Acyl-MaleimidoPropionic Acyl
FGQGTFTSDYDQXLDNKPQXDFIEWLMYGEPSSGAPPPS

SEQ ID NO: 164 - protein, 39 aa, synthetic construct
  SITE 2: note = D-serine
  MOD_RES 13: note = Aib
  MOD_RES 17: note = AminoEthoxyEthoxyAcetic Acyl-MaleimidoPropionic Acyl
FSMGTFTSDLDRXLDWKIQKDFVQWLVSGTPSSGAPPPS

SEQ ID NO: 165 - protein, 39 aa, synthetic construct
  SITE 8: note = D-serine
  SITE 12: note = D-serine
  MOD_RES 17: note = AminoEthoxyEthoxyAcetic Acyl-MaleimidoPropionic Acyl
  SITE 2: note = D-serine
YSVGTFTSDLHSYLDLKGAQDFVNWLVWGQPSSGAPPPS

SEQ ID NO: 166 - protein, 39 aa, synthetic construct
  MOD_RES 2: note = Aib
  MOD_RES 17: note = AminoEthoxyEthoxyAcetic Acyl-MaleimidoPropionic Acyl
HXIGTFTSDDVYYLDLKPQKEFVEWLGLGSPSSGAPPPS

SEQ ID NO: 167 - protein, 39 aa, synthetic construct
  MOD_RES 13: note = Aib
  MOD_RES 17: note = AminoEthoxyEthoxyAcetic Acyl-MaleimidoPropionic Acyl
FGVGTFTSDVMIXLDIKEQKEFINWLQSGEPSSGAPPPS

SEQ ID NO: 168 - protein, 39 aa, synthetic construct
  MOD_RES 2: note = Aib
  MOD_RES 11: note = Aib
  SITE 12: note = D-serine
  SITE 16: note = D-serine
  SITE 30: note = D-serine
  MOD_RES 17: note = AminoEthoxyEthoxyAcetic Acyl-MaleimidoPropionic Acyl
HXFGTFTSDHXSYLESKMAQDFINWLIIGSPSSGAPPPS

SEQ ID NO: 169 - protein, 39 aa, synthetic construct
  SITE 2: note = D-alanine
  MOD_RES 30: note = Aib
  MOD_RES 17: note = AminoEthoxyEthoxyAcetic Acyl-MaleimidoPropionic Acyl
HAGGTFTSDPAKYLDSKVQQEFVAWLAFGXPSSGAPPPS

SEQ ID NO: 170 - protein, 39 aa, synthetic construct
  SITE 2: note = D-alanine
  SITE 11: note = D-serine
  MOD_RES 13: note = Aib
  SITE 28: note = D-serine
  MOD_RES 17: note = AminoEthoxyEthoxyAcetic Acyl-MaleimidoPropionic Acyl
YANGTFTSDVSFXLEYKDQKDFIQWLASGQPSSGAPPPS

SEQ ID NO: 171 - protein, 39 aa, synthetic construct
  SITE 2: note = D-serine
  MOD_RES 20: note = Aib
  MOD_RES 17: note = AminoEthoxyEthoxyAcetic Acyl-MaleimidoPropionic Acyl
FSVGTFTSDNRQYLDVKNQXDFIQWLPWGIPSSGAPPPS

SEQ ID NO: 172 - protein, 39 aa, synthetic construct
  MOD_RES 2: note = Aib
  MOD_RES 20: note = Aib
  MOD_RES 17: note = AminoEthoxyEthoxyAcetic Acyl-MaleimidoPropionic Acyl
HXVGTFTSDKETYLDIKKAXDFVEWLSTGRPSSGAPPPS

SEQ ID NO: 173 - protein, 39 aa, synthetic construct
  MOD_RES 13: note = Aib
  MOD_RES 20: note = Aib
  MOD_RES 17: note = AminoEthoxyEthoxyAcetic Acyl-MaleimidoPropionic Acyl
FGWGTFTSDDFNXLEYKHAXEFVNWLDVGQPSSGAPPPS

SEQ ID NO: 174 - protein, 39 aa, synthetic construct
  SITE 2: note = D-alanine
  MOD_RES 13: note = Aib
  MOD_RES 17: note = AminoEthoxyEthoxyAcetic Acyl-MaleimidoPropionic Acyl
FAYGTFTSDQSYXLDYKTAKDFIQWLTKGRPSSGAPPPS

SEQ ID NO: 175 - protein, 39 aa, synthetic construct
  MOD_RES 11: note = Aib
  SITE 16: note = D-alanine
  MOD_RES 17: note = AminoEthoxyEthoxyAcetic Acyl-MaleimidoPropionic Acyl
FGPGTFTSDDXIYLEAKNAQAFIEWLFSGQPSSGAPPPS

SEQ ID NO: 176 - protein, 39 aa, synthetic construct
  MOD_RES 2: note = Aib
  SITE 11: note = D-serine
  MOD_RES 13: note = Aib
  MOD_RES 20: note = Aib
  MOD_RES 17: note = AminoEthoxyEthoxyAcetic Acyl-MaleimidoPropionic Acyl
YXIGTFTSDSSIXLDSKAQXDFIQWLLFGQPSSGAPPPS

SEQ ID NO: 177 - protein, 39 aa, synthetic construct
  SITE 2: note = D-serine
  MOD_RES 13: note = Aib
  MOD_RES 17: note = AminoEthoxyEthoxyAcetic Acyl-MaleimidoPropionic Acyl
HSGGTFTSDPYGXLEQKMAKEFVEWLMTGKPSSGAPPPS

SEQ ID NO: 178 - protein, 39 aa, synthetic construct
  SITE 2: note = D-alanine
  MOD_RES 17: note = AminoEthoxyEthoxyAcetic Acyl-MaleimidoPropionic Acyl
HAEGTFTSDYAKYLDAKRAKEFIAWLVNGGPSSGAPPPS

SEQ ID NO: 179 - protein, 39 aa, synthetic construct
  MOD_RES 2: note = Aib
  MOD_RES 13: note = Alpha-Methyl-Leucine
  MOD_RES 20: note = Aib
  MOD_RES 17: note = AminoEthoxyEthoxyAcetic Acyl-MaleimidoPropionic Acyl
YXQGTFTSDYSIXLDKKAQXAFIEYLLEGGPSSGAPPPS

SEQ ID NO: 180 - protein, 39 aa, synthetic construct; identical to SEQ ID NO: 122
  MOD_RES 16: note = AminoEthoxyEthoxyAcetic Acyl-MaleimidoPropionic Acyl

SEQ ID NO: 181 - protein, 39 aa, synthetic construct
  MOD_RES 2: note = Aib
  MOD_RES 13: note = Aib
  MOD_RES 16: note = AminoEthoxyEthoxyAcetic Acyl-MaleimidoPropionic Acyl
HXQGTFTSDYSKXLDKRRAQDFVEWLKNGGPSSGAPPPS

SEQ ID NO: 182 - protein, 39 aa, synthetic construct; identical to SEQ ID NO: 124
  MOD_RES 2: note = Aib
  MOD_RES 16: note = AminoEthoxyEthoxyAcetic Acyl-MaleimidoPropionic Acyl

SEQ ID NO: 183 - protein, 40 aa, synthetic construct
  VARIANT 1: note = X can be H, Y or F
  VARIANT 2: note = X can be G, D-Alanine, Aib or D-serine
  SITE 2: note = D-alanine or D-serine
  VARIANT 3: note = X can be A,R,N,D,E,Q,G,H,I,L,K,M,F,P,S,T,W,Y,D-alanine,Aib,D-serine or D-Lysine
  SITE 3: note = D-alanine,D-serine or D-Lysine
  MOD_RES 2: note = Aib
  MOD_RES 3: note = Aib
  VARIANT 10: note = X can be A,R,N,D,E,Q,G,H,I,L,K,M,F,P,S,T,W,Y,D-alanine,Aib,D-serine or D-Lysine
  SITE 10: note = D-alanine,D-serine or D-Lysine
  MOD_RES 10: note = Aib
  VARIANT 11: note = X can be A,R,N,D,E,Q,G,H,I,L,K,M,F,P,S,T,W,Y,D-alanine,Aib,D-serine or D-Lysine
  SITE 11: note = D-alanine,D-serine or D-Lysine
  MOD_RES 11: note = Aib
  VARIANT 12: note = X can be A,R,N,D,E,Q,G,H,I,L,K,M,F,P,S,T,W,Y,D-alanine,Aib,D-serine or D-Lysine
  SITE 12: note = D-alanine,D-serine or D-Lysine
  MOD_RES 12: note = Aib
  VARIANT 13: note = X can be Y or Aib
  MOD_RES 13: note = Aib
  VARIANT 15: note = X can be D or E
  VARIANT 16: note = X can be A,R,N,D,E,Q,G,H,I,L,K,M,F,P,S,T,W,Y,D-alanine,Aib,D-serine or D-Lysine
  SITE 16: note = D-alanine,D-serine or D-Lysine
  MOD_RES 16: note = Aib
  VARIANT 17: note = X can be A,R,N,D,E,Q,G,H,I,L,K,M,F,P,S,T,W,Y,D-alanine,Aib,D-serine or D-Lysine
  SITE 17: note = D-alanine,D-serine or D-Lysine
  MOD_RES 17: note = Aib
  VARIANT 18: note = X can be A,R,N,D,E,Q,G,H,I,L,K,M,F,P,S,T,W,Y,D-alanine,Aib,D-serine or D-Lysine
  SITE 18: note = D-alanine,D-serine or D-Lysine
  MOD_RES 18: note = Aib
  VARIANT 19: note = X can be Q or A
  VARIANT 20: note = X can be Q, K or Aib
  MOD_RES 20: note = Aib
  VARIANT 21: note = X can be D, E or A
  VARIANT 23: note = X can be V or I
  VARIANT 24: note = X can be Q, A, E or N
  VARIANT 28: note = X can be A,R,N,D,E,Q,G,H,I,L,K,M,F,P,S,T,W,Y,D-alanine,Aib,D-serine or D-Lysine
  SITE 28: note = D-alanine,D-serine or D-Lysine
  MOD_RES 28: note = Aib
  VARIANT 27: note = X can be A,R,N,D,E,Q,G,H,I,L,K,M,F,P,S,T,W,Y,D-alanine,Aib,D-serine or D-Lysine
  SITE 27: note = D-alanine,D-serine or D-Lysine
  MOD_RES 30: note = Aib
  VARIANT 30: note = X can be A,R,N,D,E,Q,G,H,I,L,K,M,F,P,S,T,W,Y,D-alanine,Aib,D-serine or D-Lysine
  SITE 30: note = D-alanine,D-serine or D-Lysine
  MOD_RES 27: note = Aib
  VARIANT 40: note = X can be K or absent
XXXGTFTSDXXXXLXXXXXXXFXXWLXXGXPSSGAPPPSX

SEQ ID NO: 184 - protein, 41 aa, synthetic construct
  VARIANT 1: note = X can be H, Y or F
  VARIANT 2: note = X can be G, D-Alanine, Aib or D-serine
  SITE 2: note = D-alanine or D-serine
  VARIANT 3: note = X can be A,R,N,D,E,Q,G,H,I,L,K,M,F,P,S,T,W,Y,D-alanine,Aib,D-serine or D-Lysine
  SITE 3: note = D-alanine,D-serine or D-Lysine
  MOD_RES 2: note = Aib
  MOD_RES 3: note = Aib
  VARIANT 10: note = X can be A,R,N,D,E,Q,G,H,I,L,K,M,F,P,S,T,W,Y,D-alanine,Aib,D-serine or D-Lysine
  SITE 10: note = D-alanine,D-serine or D-Lysine
  MOD_RES 10: note = Aib
  VARIANT 11: note = X can be A,R,N,D,E,Q,G,H,I,L,K,M,F,P,S,T,W,Y,D-alanine,Aib,D-serine or D-Lysine
  SITE 11: note = D-alanine,D-serine or D-Lysine
  MOD_RES 11: note = Aib
  VARIANT 12: note = X can be A,R,N,D,E,Q,G,H,I,L,K,M,F,P,S,T,W,Y,D-alanine,Aib,D-serine or D-Lysine
  SITE 12: note = D-alanine,D-serine or D-Lysine
  MOD_RES 12: note = Aib
  VARIANT 13: note = X can be Y, Q, N-Methyl-L-Leucine or Aib
  MOD_RES 13: note = Aib
  VARIANT 15: note = X can be D or E
  VARIANT 16: note = X can be A,R,N,D,E,Q,G,H,I,L,K,M,F,P,S,T,W,Y,D-alanine,Aib,D-serine or D-Lysine
  SITE 16: note = D-alanine,D-serine or D-Lysine
  MOD_RES 16: note = Aib
  VARIANT 17: note = X can be A,R,N,D,E,Q,G,H,I,L,K,M,F,P,S,T,W,Y,D-alanine,Aib,D-serine or D-Lysine
  SITE 17: note = D-alanine,D-serine or D-Lysine
  MOD_RES 17: note = Aib
  VARIANT 18: note = X can be A,R,N,D,E,Q,G,H,I,L,K,M,F,P,S,T,W,Y,D-alanine,Aib,D-serine or D-Lysine
  SITE 18: note = D-alanine,D-serine or D-Lysine
  MOD_RES 18: note = Aib
  VARIANT 19: note = X can be Q or A
  VARIANT 20: note = X can be Q, K, Q, R or Aib
  MOD_RES 20: note = Aib
  VARIANT 21: note = X can be D, E or A
  VARIANT 23: note = X can be V or I
  VARIANT 24: note = X can be Q, A, E or N
  VARIANT 28: note = X can be A,R,N,D,E,Q,G,H,I,L,K,M,F,P,S,T,W,Y,D-alanine,Aib,D-serine or D-Lysine
  SITE 28: note = D-alanine,D-serine or D-Lysine
  MOD_RES 28: note = Aib
  VARIANT 27: note = X can be A,R,N,D,E,Q,G,H,I,L,K,M,F,P,S,T,W,Y,D-alanine,Aib,D-serine or D-Lysine
  SITE 27: note = D-alanine,D-serine or D-Lysine
  MOD_RES 30: note = Aib
  VARIANT 30: note = X can be A,R,N,D,E,Q,G,H,I,L,K,M,F,P,S,T,W,Y,D-alanine,Aib,D-serine or D-Lysine
  SITE 30: note = D-alanine,D-serine or D-Lysine
  MOD_RES 27: note = Aib
  VARIANT 40: note = X can be K or absent
  MOD_RES 2: note = Amino-cyclobutyl-1-carboxylic acid
  MOD_RES 13: note = N-Methyl-L-Leucine
  MOD_RES 41: note = AminoEthoxyEthoxyAcetic Acid // 8-amino-3,6-dioxa-octanoic acid
XXXGTFTSDXXXXLXXXXXXXFXXWLXXGXPSSGAPPPSXK

SEQ ID NO: 185 - protein, 31 aa, synthetic construct
  MOD_RES 2: note = Aib
  MOD_RES 31: note = AminoEthoxyEthoxyAcetic Acyl-MaleimidoPropionic Acyl
HXEGTFTSDVSSYLEGQAAKEFIAWLVKGRK

SEQ ID NO: 186 - protein, 31 aa, synthetic construct; identical to SEQ ID NO: 185
  MOD_RES 2: note = Aib
  MOD_RES 31: note = AminoEthoxyEthoxyAcetic Acyl-AminoEthoxyEthoxyAcetic Acyl-MaleimidoPropionic Acyl

SEQ ID NO: 187 - protein, 31 aa, synthetic construct; identical to SEQ ID NO: 185
  MOD_RES 2: note = Aib
  MOD_RES 31: note = AminoEthoxyEthoxyAcetic Acyl-8-AminoOctanoic Acyl-MaleimidoPropionic Acyl

SEQ ID NO: 188 - protein, 31 aa, synthetic construct; identical to SEQ ID NO: 185
  MOD_RES 2: note = Aib

SEQ ID NO: 189 - protein, 32 aa, synthetic construct
  MOD_RES 2: note = Aib
  MOD_RES 32: note = AminoEthoxyEthoxyAcetic Acyl-MaleimidoPropionic Acyl
  MOD_RES 31: note = AminoEthoxyEthoxyAcetic Acid // 8-amino-3,6-dioxa-octanoic acid
HXEGTFTSDVSSYLEGQAAKEFIAWLVKGRXK

SEQ ID NO: 190 - protein, 40 aa, synthetic construct; identical to SEQ ID NO: 2
  MOD_RES 40: note = AminoEthoxyEthoxyAcetic Acyl-AminoEthoxyEthoxyAcetic Acyl-MaleimidoPropionic Acyl

SEQ ID NO: 191 - protein, 40 aa, synthetic construct; identical to SEQ ID NO: 2
  MOD_RES 40: note = AminoEthoxyEthoxyAcetic Acyl-8-AminoOctanoic Acyl-MaleimidoPropionic Acyl

SEQ ID NO: 192 - protein, 40 aa, synthetic construct; identical to SEQ ID NO: 2

SEQ ID NO: 193 - protein, 41 aa, synthetic construct
  MOD_RES 40: note = AminoEthoxyEthoxyAcetic Acid // 8-amino-3,6-dioxa-octanoic acid
  MOD_RES 41: note = AminoEthoxyEthoxyAcetic Acyl-MaleimidoPropionic Acyl
HGEGTFTSDLSKQMEEEAVRLFIEWLKNGGPSSGAPPPSXK

SEQ ID NO: 194 - protein, 40 aa, synthetic construct
  SITE 2: note = D-serine
  MOD_RES 40: note = AminoEthoxyEthoxyAcetic Acyl-MaleimidoPropionic Acyl
HSQGTFTSDYSKYLDSRRAQDFVQWLMNTGPSSGAPPPSK

SEQ ID NO: 195 - protein, 40 aa, synthetic construct; identical to SEQ ID NO: 194
  SITE 2: note = D-serine
  MOD_RES 40: note = AminoEthoxyEthoxyAcetic Acyl-AminoEthoxyEthoxyAcetic Acyl-MaleimidoPropionic Acyl

SEQ ID NO: 196 - protein, 40 aa, synthetic construct; identical to SEQ ID NO: 194
  SITE 2: note = D-serine
  MOD_RES 40: note = AminoEthoxyEthoxyAcetic Acyl-8-AminoOctanoic Acyl-MaleimidoPropionic Acyl

SEQ ID NO: 197 - protein, 40 aa, synthetic construct; identical to SEQ ID NO: 194
  SITE 2: note = D-serine

SEQ ID NO: 198 - protein, 41 aa, synthetic construct
  SITE 2: note = D-serine
  MOD_RES 40: note = AminoEthoxyEthoxyAcetic Acid // 8-amino-3,6-dioxa-octanoic acid
  MOD_RES 41: note = AminoEthoxyEthoxyAcetic Acyl-MaleimidoPropionic Acyl
HSQGTFTSDYSKYLDSRRAQDFVQWLMNTGPSSGAPPPSXK

SEQ ID NO: 199 - protein, 40 aa, synthetic construct
  SITE 2: note = D-alanine
  MOD_RES 40: note = AminoEthoxyEthoxyAcetic Acyl-MaleimidoPropionic Acyl
YAEGTFISDYSIAMDKIHQQDFVNWLLAQGPSSGAPPPSK

SEQ ID NO: 200 - protein, 40 aa, synthetic construct; identical to SEQ ID NO: 199
  SITE 2: note = D-alanine
  MOD_RES 40: note = AminoEthoxyEthoxyAcetic Acyl-AminoEthoxyEthoxyAcetic Acyl-MaleimidoPropionic Acyl

SEQ ID NO: 201 - protein, 40 aa, synthetic construct; identical to SEQ ID NO: 199
  SITE 2: note = D-alanine
  MOD_RES 40: note = AminoEthoxyEthoxyAcetic Acyl-8-AminoOctanoic Acyl-MaleimidoPropionic Acyl

SEQ ID NO: 202 - protein, 40 aa, synthetic construct; identical to SEQ ID NO: 199
  SITE 2: note = D-alanine

SEQ ID NO: 203 - protein, 41 aa, synthetic construct
  SITE 2: note = D-alanine
  MOD_RES 40: note = AminoEthoxyEthoxyAcetic Acid // 8-amino-3,6-dioxa-octanoic acid
  MOD_RES 41: note = AminoEthoxyEthoxyAcetic Acyl-MaleimidoPropionic Acyl
YAEGTFISDYSIAMDKIHQQDFVNWLLAQGPSSGAPPPSXK

SEQ ID NO: 204 - protein, 30 aa, synthetic construct
  MOD_RES 20: note = AminoEthoxyEthoxyAcetic Acyl-MaleimidoPropionic Acyl
HAEGTFTSDVSSYLEGQAAKEFIAWLVRGR

SEQ ID NO: 205 - protein, 31 aa, synthetic construct
  MOD_RES 20: note = AminoEthoxyEthoxyAcetic Acyl-MaleimidoPropionic Acyl
  MOD_RES 2: note = Aib
HXEGTFTSDVSSYLEGQAAKEFIAWLVRGRG

SEQ ID NO: 206 - protein, 31 aa, synthetic construct
  MOD_RES 24: note = AminoEthoxyEthoxyAcetic Acyl-MaleimidoPropionic Acyl
HVEGTFTSDVSSYLEEQAAREFIKWLVRGRG

SEQ ID NO: 207 - protein, 40 aa, synthetic construct; identical to SEQ ID NO: 7
  SITE 2: note = D-alanine
  MOD_RES 40: note = AminoEthoxyEthoxyAcetic Acyl-AminoEthoxyEthoxyAcetic Acyl-MaleimidoPropionic Acyl

SEQ ID NO: 208 - protein, 40 aa, synthetic construct; identical to SEQ ID NO: 7
  SITE 2: note = D-alanine
  MOD_RES 40: note = AminoEthoxyEthoxyAcetic Acyl-8-AminoOctanoic Acyl-MaleimidoPropionic Acyl

SEQ ID NO: 209 - protein, 40 aa, synthetic construct; identical to SEQ ID NO: 7
  SITE 2: note = D-alanine

SEQ ID NO: 210 - protein, 41 aa, synthetic construct
  SITE 2: note = D-alanine
  MOD_RES 40: note = AminoEthoxyEthoxyAcetic Acid // 8-amino-3,6-dioxa-octanoic acid
  MOD_RES 41: note = AminoEthoxyEthoxyAcetic Acyl-MaleimidoPropionic Acyl
HAEGTFTSDVSSYLEGQAAKEFIAWLVNGGPSSGAPPPSXK

SEQ ID NO: 211 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDVASYLEGQAAKEFIAWLVNGGPSSGAPPPSK

SEQ ID NO: 212 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
YXEGTFTSDYSIYLDKQAAXEFVNWLLAGGPSSGAPPPSK

SEQ ID NO: 213 - protein, 34 aa, synthetic construct
  MOD_RES 2: note = Aib
  MOD_RES 20: note = AminoEthoxyEthoxyAcetic Acyl-MaleimidoPropionic Acyl
HXQGTFTSDYSKYLDEKKAKEFVEWLLEGGPSSG

SEQ ID NO: 214 - protein, 29 aa, synthetic construct
  MOD_RES 2: note = Aib
  MOD_RES 17: note = AminoEthoxyEthoxyAcetic Acyl-MaleimidoPropionic Acyl
HXQGTFTSDYSKYLDEKAAKEFIQWLLQT

SEQ ID NO: 215 - protein, 30 aa, synthetic construct
  MOD_RES 2: note = Aib
HXQGTFTSDYSKYLDEKRAKEFVQWLMNTC

SEQ ID NO: 216 - protein, 29 aa, synthetic construct
  SITE 2: note = D-serine
  MOD_RES 10: note = AminoEthoxyEthoxyAcetic Acyl-MaleimidoPropionic Acyl
HSQGTFTSDKSKYLDARAAQDFVQWLLDT

SEQ ID NO: 217 - protein, 29 aa, synthetic construct
  MOD_RES 24: note = AminoEthoxyEthoxyAcetic Acyl-MaleimidoPropionic Acyl
  MOD_RES 2: note = Amino-cyclobutyl-1-carboxylic acid
HXQGTFTSDYSKYLDERAAKDFIKWLESA

SEQ ID NO: 218 - protein, 39 aa, synthetic construct
  SITE 2: note = D-serine
  MOD_RES 14: note = AminoEthoxyEthoxyAcetic Acyl-MaleimidoPropionic Acyl
HSQGTFTSDLSKQKESKAAQDFIEWLKAGGPSSGAPPPS

SEQ ID NO: 219 - protein, 30 aa, synthetic construct
  MOD_RES 10: note = AminoEthoxyEthoxyAcetic Acyl-MaleimidoPropionic Acyl
HSQGTFTSDKSEYLDSERARDFVAWLEAGG

SEQ ID NO: 220 - protein, 39 aa, synthetic construct
  MOD_RES 2: note = Aib
  MOD_RES 10: note = AminoEthoxyEthoxyAcetic Acyl-MaleimidoPropionic Acyl
HXQGTFTSDKSKYLDERAAQDFVQWLLDGGPSSGAPPPS

SEQ ID NO: 221 - protein, 39 aa, synthetic construct
  MOD_RES 10: note = AminoEthoxyEthoxyAcetic Acyl-MaleimidoPropionic Acyl
HSQGTFTSDKSKYLDERRAQDFVQWLLDGGPSSGAPPPS

SEQ ID NO: 222 - protein, 39 aa, synthetic construct
  MOD_RES 2: note = Aib
  MOD_RES 20: note = Aib
  MOD_RES 34: note = Aib
  MOD_RES 14: note = AminoEthoxyEthoxyAcetic Acyl-MaleimidoPropionic Acyl
HXHGTGTSDLSKLKEEQRQXEFIEWLKAAGPPSXKPPPK

SEQ ID NO: 223 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFIAWLVNGGPSSGAPPPSK

SEQ ID NO: 224 - protein, 40 aa, synthetic construct; identical to SEQ ID NO: 223
  SITE 2: note = D-alanine
  MOD_RES 40: note = AminoEthoxyEthoxyAcetic Acyl-AminoEthoxyEthoxyAcetic Acyl-MaleimidoPropionic Acyl

SEQ ID NO: 225 - protein, 40 aa, synthetic construct; identical to SEQ ID NO: 223
  SITE 2: note = D-alanine
  MOD_RES 40: note = AminoEthoxyEthoxyAcetic Acyl-8-AminoOctanoic Acyl-MaleimidoPropionic Acyl

SEQ ID NO: 226 - protein, 40 aa, synthetic construct; identical to SEQ ID NO: 223
  SITE 2: note = D-alanine

SEQ ID NO: 227 - protein, 40 aa, synthetic construct; identical to SEQ ID NO: 69
  SITE 2: note = D-alanine
  MOD_RES 40: note = AminoEthoxyEthoxyAcetic Acyl-AminoEthoxyEthoxyAcetic Acyl-MaleimidoPropionic Acyl

SEQ ID NO: 228 - protein, 40 aa, synthetic construct; identical to SEQ ID NO: 69
  MOD_RES 40: note = AminoEthoxyEthoxyAcetic Acyl-8-AminoOctanoic Acyl-MaleimidoPropionic Acyl
  SITE 2: note = D-alanine

SEQ ID NO: 229 - protein, 40 aa, synthetic construct; identical to SEQ ID NO: 69
  SITE 2: note = D-alanine

SEQ ID NO: 230 - protein, 41 aa, synthetic construct
  SITE 2: note = D-alanine
  MOD_RES 40: note = AminoEthoxyEthoxyAcetic Acid // 8-amino-3,6-dioxa-octanoic acid
  MOD_RES 41: note = AminoEthoxyEthoxyAcetic Acyl-MaleimidoPropionic Acyl
HAEGTFTSDYAKYLDARRAKEFIAWLVNGGPSSGAPPPSXK

SEQ ID NO: 231 - protein, 42 aa, synthetic construct; identical to SEQ ID NO: 5

SEQ ID NO: 232 - protein, 41 aa, synthetic construct
  SITE 2: note = D-alanine
  MOD_RES 40: note = AminoEthoxyEthoxyAcetic Acid // 8-amino-3,6-dioxa-octanoic acid
  MOD_RES 41: note = AminoEthoxyEthoxyAcetic Acyl-MaleimidoPropionic Acyl
HAEGTFTSDVASYLEGQAAKEFIAWLVNGGPSSGAPPPSXK

SEQ ID NO: 233 - protein, 31 aa, synthetic construct; identical to SEQ ID NO: 205
  MOD_RES 2: note = Aib
  MOD_RES 20: note = AminoEthoxyEthoxyAcetic Acyl-AminoEthoxyEthoxyAcetic Acyl-MaleimidoPropionic Acyl

SEQ ID NO: 234 - protein, 32 aa, synthetic construct
  MOD_RES 2: note = Aib
  MOD_RES 32: note = AminoEthoxyEthoxyAcetic Acyl-MaleimidoPropionic Acyl
HXEGTFTSDVSSYLEGQAAKEFIAWLVRGRGK

SEQ ID NO: 235 - protein, 32 aa, synthetic construct
  MOD_RES 2: note = Aib
  MOD_RES 32: note = AminoEthoxyEthoxyAcetic Acyl-8-AminoOctanoic Acyl-MaleimidoPropionic Acyl
  MOD_RES 20: note = Aib
HXEGTFTSDVSSYLEGQAAXEFIAWLVRGRGK

SEQ ID NO: 236 - protein, 39 aa, synthetic construct; identical to SEQ ID NO: 125
  MOD_RES 2: note = Aib
  MOD_RES 13: note = Aib
  MOD_RES 20: note = AminoEthoxyEthoxyAcetic Acyl-8-AminoOctanoic Acyl-MaleimidoPropionic Acyl

SEQ ID NO: 237 - protein, 39 aa, synthetic construct; identical to SEQ ID NO: 125
  MOD_RES 2: note = Aib
  MOD_RES 13: note = Aib
  MOD_RES 20: note = AminoEthoxyEthoxyAcetic Acyl-AminoEthoxyEthoxyAcetic Acyl-MaleimidoPropionic Acyl

SEQ ID NO: 238 - protein, 40 aa, synthetic construct; identical to SEQ ID NO: 14
  MOD_RES 2: note = Aib
  MOD_RES 20: note = Aib
  MOD_RES 40: note = AminoEthoxyEthoxyAcetic Acyl-AminoEthoxyEthoxyAcetic Acyl-MaleimidoPropionic Acyl

SEQ ID NO: 239 - protein, 40 aa, synthetic construct; identical to SEQ ID NO: 14
  MOD_RES 2: note = Aib
  MOD_RES 20: note = Aib
  MOD_RES 40: note = AminoEthoxyEthoxyAcetic Acyl-8-AminoOctanoic Acyl-AminoEthoxyEthoxyAcetic Acyl-MaleimidoPropionic Acyl

SEQ ID NO: 240 - protein, 34 aa, synthetic construct; identical to SEQ ID NO: 213
  MOD_RES 2: note = Aib
  MOD_RES 20: note = AminoEthoxyEthoxyAcetic Acyl-AminoEthoxyEthoxyAcetic Acyl-MaleimidoPropionic Acyl

SEQ ID NO: 241 - protein, 34 aa, synthetic construct; identical to SEQ ID NO: 213
  MOD_RES 2: note = Aib
  MOD_RES 20: note = AminoEthoxyEthoxyAcetic Acyl-8-AminoOctanoic Acyl-MaleimidoPropionic Acyl

SEQ ID NO: 242 - protein, 34 aa, synthetic construct; identical to SEQ ID NO: 213
  MOD_RES 2: note = Aib
  MOD_RES 20: note = AminoEthoxyEthoxyAcetic Acyl-8-AminoOctanoic Acyl-AminoEthoxyEthoxyAcetic Acyl-MaleimidoPropionic Acyl

SEQ ID NO: 243 - protein, 35 aa, synthetic construct
  MOD_RES 2: note = Aib
  MOD_RES 35: note = AminoEthoxyEthoxyAcetic Acyl-MaleimidoPropionic Acyl
HXQGTFTSDYSKYLDEKKAKEFVEWLLEGGPSSGK

SEQ ID NO: 244 - protein, 29 aa, synthetic construct
  MOD_RES 2: note = Aib
  MOD_RES 17: note = AminoEthoxyEthoxyAcetic Acyl-AminoEthoxyEthoxyAcetic Acyl-MaleimidoPropionic Acyl
HXQGTFTSDYSKYLDEKAAKEFIQWLLQT

SEQ ID NO: 245 - protein, 29 aa, synthetic construct
  MOD_RES 2: note = Aib
  MOD_RES 17: note = AminoEthoxyEthoxyAcetic Acyl-8-AminoOctanoic Acyl-MaleimidoPropionic Acyl
HXQGTFTSDYSKYLDEKAAKEFIQWLLQT

SEQ ID NO: 246 - protein, 39 aa, synthetic construct; identical to SEQ ID NO: 218
  SITE 2: note = D-serine
  MOD_RES 14: note = AminoEthoxyEthoxyAcetic Acyl-AminoEthoxyEthoxyAcetic Acyl-MaleimidoPropionic Acyl

SEQ ID NO: 247 - protein, 39 aa, synthetic construct; identical to SEQ ID NO: 218
  SITE 2: note = D-serine
  MOD_RES 14: note = AminoEthoxyEthoxyAcetic Acyl-8-AminoOctanoic Acyl-AminoEthoxyEthoxyAcetic Acyl-MaleimidoPropionic Acyl

SEQ ID NO: 248 - protein, 40 aa, synthetic construct
  SITE 2: note = D-serine
  MOD_RES 40: note = AminoEthoxyEthoxyAcetic Acyl-MaleimidoPropionic Acyl
HSQGTFTSDLSKQLESKAAQDFIEWLKAGGPSSGAPPPSK

SEQ ID NO: 249 - protein, 40 aa, synthetic construct; identical to SEQ ID NO: 248
  SITE 2: note = D-serine
  MOD_RES 40: note = AminoEthoxyEthoxyAcetic Acyl-8-AminoOctanoic Acyl-MaleimidoPropionic Acyl

SEQ ID NO: 250 - protein, 31 aa, synthetic construct
  MOD_RES 31: note = AminoEthoxyEthoxyAcetic Acyl-Bromo-MaleimidoPropionic Acyl//8-amino-3,6-dioxa-octanoic acyl-3-(3-Bromo-2,5-dioxopyrrol-1-yl)propanoic acyl
HSQGTFTSDLSEYLDSERARDFVAWLEAGGK

SEQ ID NO: 251 - protein, 39 aa, synthetic construct; identical to SEQ ID NO: 179
  MOD_RES 2: note = Aib
  MOD_RES 13: note = Alpha-Methyl-Leucine
  MOD_RES 17: note = AminoEthoxyEthoxyAcetic Acyl-AminoEthoxyEthoxyAcetic Acyl-MaleimidoPropionic Acyl
  MOD_RES 20: note = Aib

SEQ ID NO: 252 - protein, 39 aa, synthetic construct; identical to SEQ ID NO: 179
  MOD_RES 2: note = Aib
  MOD_RES 20: note = Aib
  MOD_RES 13: note = Alpha-Methyl-Leucine
  MOD_RES 17: note = AminoEthoxyEthoxyAcetic Acyl-8-AminoOctanoic Acyl-MaleimidoPropionic Acyl

SEQ ID NO: 253 - protein, 39 aa, synthetic construct; identical to SEQ ID NO: 179
  MOD_RES 2: note = Aib
  MOD_RES 20: note = Aib
  MOD_RES 13: note = Alpha-Methyl-Leucine
  MOD_RES 17: note = AminoEthoxyEthoxyAcetic Acyl-8-AminoOctanoic Acyl-AminoEthoxyEthoxyAcetic Acyl-MaleimidoPropionic Acyl

SEQ ID NO: 254 - protein, 40 aa, synthetic construct; identical to SEQ ID NO: 67
  MOD_RES 2: note = Aib
  MOD_RES 13: note = Alpha-Methyl-Leucine
  MOD_RES 20: note = Aib
  MOD_RES 40: note = AminoEthoxyEthoxyAcetic Acyl-AminoEthoxyEthoxyAcetic Acyl-MaleimidoPropionic Acyl

SEQ ID NO: 255 - protein, 40 aa, synthetic construct; identical to SEQ ID NO: 67
  MOD_RES 2: note = Aib
  MOD_RES 20: note = Aib
  MOD_RES 13: note = Alpha-Methyl-Leucine
  MOD_RES 40: note = AminoEthoxyEthoxyAcetic Acyl-8-AminoOctanoic Acyl-MaleimidoPropionic Acyl

SEQ ID NO: 256 - protein, 40 aa, synthetic construct; identical to SEQ ID NO: 67
  MOD_RES 2: note = Aib
  MOD_RES 20: note = Aib
  MOD_RES 13: note = Alpha-Methyl-Leucine
  MOD_RES 40: note = AminoEthoxyEthoxyAcetic Acyl-8-AminoOctanoic Acyl-AminoEthoxyEthoxyAcetic Acyl-MaleimidoPropionic Acyl

SEQ ID NO: 257 - protein, 39 aa, synthetic construct; identical to SEQ ID NO: 221
  MOD_RES 10: note = AminoEthoxyEthoxyAcetic Acyl-AminoEthoxyEthoxyAcetic Acyl-MaleimidoPropionic Acyl

SEQ ID NO: 258 - protein, 39 aa, synthetic construct; identical to SEQ ID NO: 221
  MOD_RES 10: note = AminoEthoxyEthoxyAcetic Acyl-8-AminoOctanoic Acyl-MaleimidoPropionic Acyl

SEQ ID NO: 259 - protein, 40 aa, synthetic construct
  MOD_RES 40: note = AminoEthoxyEthoxyAcetic Acyl-MaleimidoPropionic Acyl
HSQGTFTSDYSKYLDERRAQDFVQWLLDGGPSSGAPPPSK

SEQ ID NO: 260 - protein, 40 aa, synthetic construct; identical to SEQ ID NO: 259
  MOD_RES 40: note = AminoEthoxyEthoxyAcetic Acyl-Bromo-MaleimidoPropionic Acyl//8-amino-3,6-dioxa-octanoic acyl-3-(3-Bromo-2,5-dioxopyrrol-1-yl)propanoic acyl

SEQ ID NO: 261 - protein, 39 aa, synthetic construct; identical to SEQ ID NO: 222
  MOD_RES 2: note = Aib
  MOD_RES 34: note = Aib
  MOD_RES 14: note = AminoEthoxyEthoxyAcetic Acyl-AminoEthoxyEthoxyAcetic Acyl-MaleimidoPropionic Acyl
  SITE 29: note = D-alanine
  MOD_RES 20: note = Aib

SEQ ID NO: 262 - protein, 39 aa, synthetic construct
  MOD_RES 2: note = Aib
  MOD_RES 20: note = Aib
  MOD_RES 34: note = Aib
  SITE 29: note = D-alanine
  MOD_RES 39: note = AminoEthoxyEthoxyAcetic Acyl-MaleimidoPropionic Acyl
HXHGTGTSDLSKLLEEQRQXEFIEWLKAAGPPSXKPPPK

SEQ ID NO: 263 - protein, 40 aa, synthetic construct
  MOD_RES 2: note = Aib
  MOD_RES 20: note = Aib
  MOD_RES 34: note = Aib
  SITE 29: note = D-alanine
  MOD_RES 39: note = AminoEthoxyEthoxyAcetic Acid // 8-amino-3,6-dioxa-octanoic acid
  MOD_RES 40: note = AminoEthoxyEthoxyAcetic Acyl-MaleimidoPropionic Acyl
HXHGTGTSDLSKLLEEQRQXEFIEWLKAAGPPSXKPPPXK

SEQ ID NO: 264 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
YAEGTFTSDYAKYLDARRAXEFIAWLVNGGPSSGAPPPSK

SEQ ID NO: 265 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
YXEGTFTSDYAKYLDARRAXEFIAWLVNGGPSSGAPPPSK

SEQ ID NO: 266 - protein, 40 aa, synthetic construct
  MOD_RES 2: note = Aib
  MOD_RES 13: note = Aib
  MOD_RES 20: note = Aib
  MOD_RES 40: note = AminoEthoxyEthoxyAcetic Acyl-MaleimidoPropionic Acyl
YXEGTFTSDYSIXLDKRRAXEFIAWLVNGGPSSGAPPPSK

SEQ ID NO: 267 - protein, 40 aa, synthetic construct
  SITE 2: note = D-alanine
  MOD_RES 13: note = Aib
  MOD_RES 20: note = Aib
  MOD_RES 40: note = AminoEthoxyEthoxyAcetic Acyl-MaleimidoPropionic Acyl
HAEGTFTSDYSIXLDKRRAXEFIAWLVNGGPSSGAPPPSK

SEQ ID NO: 268 - protein, 40 aa, synthetic construct
  SITE 2: note = D-alanine
  MOD_RES 13: note = Aib
  MOD_RES 20: note = Aib
  MOD_RES 40: note = AminoEthoxyEthoxyAcetic Acyl-MaleimidoPropionic Acyl
YAEGTFTSDYSIXLDKRRAXEFIAWLVNGGPSSGAPPPSK

SEQ ID NO: 269 - protein, 40 aa, synthetic construct
  MOD_RES 2: note = Aib
  MOD_RES 13: note = Aib
  MOD_RES 20: note = Aib
  MOD_RES 40: note = AminoEthoxyEthoxyAcetic Acyl-MaleimidoPropionic Acyl
YXEGTFTSDYSIXLDKIAQXAFIAWLVNGGPSSGAPPPSK

SEQ ID NO: 270 - protein, 40 aa, synthetic construct
  SITE 2: note = D-alanine
  MOD_RES 13: note = Aib
  MOD_RES 20: note = Aib
  MOD_RES 40: note = AminoEthoxyEthoxyAcetic Acyl-MaleimidoPropionic Acyl
HAEGTFTSDYSIXLDKIAQXAFIAWLVNGGPSSGAPPPSK

SEQ ID NO: 271 - protein, 40 aa, synthetic construct
  SITE 2: note = D-alanine
  MOD_RES 13: note = Aib
  MOD_RES 20: note = Aib
  MOD_RES 40: note = AminoEthoxyEthoxyAcetic Acyl-MaleimidoPropionic Acyl
YAEGTFTSDYSIXLDKIAQXAFIAWLVNGGPSSGAPPPSK

SEQ ID NO: 272 - protein, 40 aa, synthetic construct
  SITE 2: note = D-alanine
  MOD_RES 13: note = Aib
  MOD_RES 20: note = Aib
  MOD_RES 40: note = AminoEthoxyEthoxyAcetic Acyl-MaleimidoPropionic Acyl
HAEGTFTSDYSIXLDKIAQXAFVQWLIAGGPSSGAPPPSK

SEQ ID NO: 273 - protein, 40 aa, synthetic construct
  SITE 2: note = D-alanine
  MOD_RES 13: note = Aib
  MOD_RES 20: note = Aib
  MOD_RES 40: note = AminoEthoxyEthoxyAcetic Acyl-MaleimidoPropionic Acyl
YAEGTFTSDYSIXLDKIAQXAFVQWLIAGGPSSGAPPPSK

SEQ ID NO: 274 - protein, 41 aa, synthetic construct
  MOD_RES 2: note = Aib
  MOD_RES 13: note = Aib
  MOD_RES 20: note = Aib
  MOD_RES 40: note = AminoEthoxyEthoxyAcetic Acid // 8-amino-3,6-dioxa-octanoic acid
  MOD_RES 41: note = AminoEthoxyEthoxyAcetic Acyl-MaleimidoPropionic Acyl
YXEGTFTSDYSIXLDKIAQXAFVQWLIAGGPSSGAPPPSXK

SEQ ID NO: 275 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
HAEGTFTSDYAKYLDARRAXEFVQWLIAGGPSSGAPPPSK

SEQ ID NO: 276 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
YAEGTFTSDYAKYLDARRAXEFVQWLIAGGPSSGAPPPSK

SEQ ID NO: 277 - protein, 40 aa, synthetic construct
  MOD_RES 2: note = Aib
  MOD_RES 40: note = AminoEthoxyEthoxyAcetic Acyl-MaleimidoPropionic Acyl
  MOD_RES 20: note = Aib
YXEGTFTSDYAKYLDARRAXEFVQWLIAGGPSSGAPPPSK

SEQ ID NO: 278 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
HAEGTFTSDYAKYLDKIAQXAFVQWLIAGGPSSGAPPPSK

SEQ ID NO: 279 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
YAEGTFTSDYAKYLDKIAQXAFVQWLIAGGPSSGAPPPSK

SEQ ID NO: 280 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
YXEGTFTSDYAKYLDKIAQXAFVQWLIAGGPSSGAPPPSK

SEQ ID NO: 281 - protein, 40 aa, synthetic construct
  MOD_RES 2: note = Aib
  MOD_RES 40: note = AminoEthoxyEthoxyAcetic Acyl-MaleimidoPropionic Acyl
YXQGTFTSDYAKYLDARRAKEFIAWLVNGGPSSGAPPPSK

SEQ ID NO: 282 - protein, 40 aa, synthetic construct
  SITE 2: note = D-alanine
  MOD_RES 40: note = AminoEthoxyEthoxyAcetic Acyl-MaleimidoPropionic Acyl
YAQGTFTSDYAKYLDARRAKEFIAWLVNGGPSSGAPPPSK

SEQ ID NO: 283 - protein, 40 aa, synthetic construct
  MOD_RES 2: note = Aib
  MOD_RES 13: note = Alpha-Methyl-Leucine
  MOD_RES 40: note = AminoEthoxyEthoxyAcetic Acyl-MaleimidoPropionic Acyl
YXQGTFTSDYSIXLDKRRAKEFIAWLVNGGPSSGAPPPSK

SEQ ID NO: 284 - protein, 40 aa, synthetic construct
  SITE 2: note = D-alanine
  MOD_RES 13: note = Alpha-Methyl-Leucine
  MOD_RES 40: note = AminoEthoxyEthoxyAcetic Acyl-MaleimidoPropionic Acyl
YAQGTFTSDYSIXLDKRRAKEFIAWLVNGGPSSGAPPPSK

SEQ ID NO: 285 - protein, 40 aa, synthetic construct
  SITE 2: note = D-alanine
  MOD_RES 13: note = Alpha-Methyl-Leucine
  MOD_RES 40: note = AminoEthoxyEthoxyAcetic Acyl-MaleimidoPropionic Acyl
HAQGTFTSDYSIXLDKRRAKEFIAWLVNGGPSSGAPPPSK

SEQ ID NO: 286 - protein, 40 aa, synthetic construct
  MOD_RES 2: note = Aib
  MOD_RES 13: note = Alpha-Methyl-Leucine
  MOD_RES 20: note = Aib
  MOD_RES 40: note = AminoEthoxyEthoxyAcetic Acyl-MaleimidoPropionic Acyl
YXQGTFTSDYSIXLDKIAQXAFIAWLVNGGPSSGAPPPSK

SEQ ID NO: 287 - protein, 40 aa, synthetic construct
  SITE 2: note = D-alanine
  MOD_RES 13: note = Alpha-Methyl-Leucine
  MOD_RES 20: note = Aib
  MOD_RES 40: note = AminoEthoxyEthoxyAcetic Acyl-MaleimidoPropionic Acyl
YAQGTFTSDYSIXLDKIAQXAFIAWLVNGGPSSGAPPPSK

SEQ ID NO: 288 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
  MOD_RES 13: note = Alpha-Methyl-Leucine
HAQGTFTSDYSIXLDKIAQXAFIAWLVNGGPSSGAPPPSK

SEQ ID NO: 289 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
  MOD_RES 13: note = Alpha-Methyl-Leucine
YAQGTFTSDYSIXLDKIAQXAFIEYLLEGGPSSGAPPPSK

SEQ ID NO: 290 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
  MOD_RES 13: note = Alpha-Methyl-Leucine
HAQGTFTSDYSIXLDKIAQXAFIEYLLEGGPSSGAPPPSK

SEQ ID NO: 291 - protein, 41 aa, synthetic construct
  MOD_RES 2: note = Aib
  MOD_RES 20: note = Aib
  MOD_RES 13: note = Alpha-Methyl-Leucine
  MOD_RES 40: note = AminoEthoxyEthoxyAcetic Acid // 8-amino-3,6-dioxa-octanoic acid
  MOD_RES 41: note = AminoEthoxyEthoxyAcetic Acyl-MaleimidoPropionic Acyl
YXQGTFTSDYSIXLDKIAQXAFIEYLLEGGPSSGAPPPSXK

SEQ ID NO: 292 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFIEYLLEGGPSSGAPPPSK

SEQ ID NO: 293 - protein, 40 aa, synthetic construct
  MOD_RES 40: note = AminoEthoxyEthoxyAcetic Acyl-MaleimidoPropionic Acyl
  MOD_RES 2: note = Aib
HXQGTFTSDYAKYLDARRAKEFIEYLLEGGPSSGAPPPSK

SEQ ID NO: 294 - protein, 40 aa, synthetic construct
  MOD_RES 2: note = Aib
  MOD_RES 40: note = AminoEthoxyEthoxyAcetic Acyl-MaleimidoPropionic Acyl
YXQGTFTSDYAKYLDARRAKEFIEYLLEGGPSSGAPPPSK

SEQ ID NO: 295 - protein, 40 aa, synthetic construct
  SITE 2: note = D-alanine
  MOD_RES 40: note = AminoEthoxyEthoxyAcetic Acyl-MaleimidoPropionic Acyl
YAQGTFTSDYAKYLDARRAKEFIEYLLEGGPSSGAPPPSK

SEQ ID NO: 296 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
HAQGTFTSDYAKYLDKIAQXAFIEYLLEGGPSSGAPPPSK

SEQ ID NO: 297 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
HXQGTFTSDYAKYLDKIAQXAFIEYLLEGGPSSGAPPPSK

SEQ ID NO: 298 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
YXQGTFTSDYAKYLDKIAQXAFIEYLLEGGPSSGAPPPSK

SEQ ID NO: 299 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
YAQGTFTSDYAKYLDKIAQXAFIEYLLEGGPSSGAPPPSK

SEQ ID NO: 300 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
  MOD_RES 13: note = Alpha-Methyl-Leucine
HXQGTFTSDYSIXLDKIAQXAFIEYLLEGGPSSGAPPPSK

SEQ ID NO: 301 - protein, 40 aa, synthetic construct; identical to SEQ ID NO: 223
  SITE 2: note = D-alanine
  MOD_RES 40: note = AminoEthoxyEthoxyAcetic Acyl-8-AminoOctanoic Acyl-AminoEthoxyEthoxyAcetic Acyl-MaleimidoPropionic Acyl

SEQ ID NO: 302 - protein, 39 aa, synthetic construct; identical to SEQ ID NO: 178
  SITE 2: note = D-alanine
  MOD_RES 17: note = AminoEthoxyEthoxyAcetic Acyl-MaleimidoPropionic Acyl

SEQ ID NO: 303 - protein, 39 aa, synthetic construct; identical to SEQ ID NO: 178
  SITE 2: note = D-alanine
  MOD_RES 17: note = AminoEthoxyEthoxyAcetic Acyl-AminoEthoxyEthoxyAcetic Acyl-MaleimidoPropionic Acyl

SEQ ID NO: 304 - protein, 39 aa, synthetic construct; identical to SEQ ID NO: 178
  SITE 2: note = D-alanine
  MOD_RES 17: note = AminoEthoxyEthoxyAcetic Acyl-AminoEthoxyEthoxyAcetic Acyl-8-AminoOctanoic Acyl-MaleimidoPropionic Acyl

SEQ ID NO: 305 - protein, 39 aa, synthetic construct; identical to SEQ ID NO: 178
  SITE 2: note = D-alanine
  MOD_RES 17: note = AminoEthoxyEthoxyAcetic Acyl-8-AminoOctanoic Acyl-MaleimidoPropionic Acyl

SEQ ID NO: 306 - protein, 39 aa, synthetic construct; identical to SEQ ID NO: 127
  SITE 2: note = D-alanine
  MOD_RES 20: note = AminoEthoxyEthoxyAcetic Acyl-AminoEthoxyEthoxyAcetic Acyl-MaleimidoPropionic Acyl

SEQ ID NO: 307 - protein, 39 aa, synthetic construct; identical to SEQ ID NO: 127
  SITE 2: note = D-alanine
  MOD_RES 20: note = AminoEthoxyEthoxyAcetic Acyl-AminoEthoxyEthoxyAcetic Acyl-8-AminoOctanoic Acyl-MaleimidoPropionic Acyl

SEQ ID NO: 308 - protein, 39 aa, synthetic construct; identical to SEQ ID NO: 127
  SITE 2: note = D-alanine
  MOD_RES 20: note = AminoEthoxyEthoxyAcetic Acyl-8-AminoOctanoic Acyl-MaleimidoPropionic Acyl

SEQ ID NO: 309 - protein, 40 aa, synthetic construct; identical to SEQ ID NO: 223
  SITE 2: note = D-alanine
  MOD_RES 40: note = AminoEthoxyEthoxyAcetic Acyl-AminoEthoxyEthoxyAcetic Acyl-gamma-glutamic acid 1-Amido Eicosanedioic acyl // 2-[(20-oxoicosanoyl)amino]-5 -[2-[2-[2-[2-[2-(carboxymethoxy)ethoxy]ethylamino]-2-oxoet hoxy]ethoxy]ethylamino]-5-oxopentanoic acyl

SEQ ID NO: 310 - protein, 40 aa, synthetic construct; identical to SEQ ID NO: 223
  SITE 2: note = D-alanine
  MOD_RES 40: note = AminoEthoxyEthoxyAcetic Acyl-gamma-glutamic acid 1-Amido Eicosanedioic acyl // 2-[(20-oxoicosanoyl)amino]-5 -[2-[2-[2-[2-[2-(carboxymethoxy)ethoxy]ethylamino]-2-oxoet hoxy]ethoxy]ethylamino]-5-oxopentanoic acyl

SEQ ID NO: 311 - protein, 40 aa, synthetic construct; identical to SEQ ID NO: 223
  SITE 2: note = D-alanine
  MOD_RES 40: note = AminoEthoxyEthoxyAcetic Acyl-AminoEthoxyEthoxyAcetic Acyl-gamma-glutamic acid 1-Amido 19-Phosphatidyl Nonadecanoic acyl

SEQ ID NO: 312 - protein, 40 aa, synthetic construct
  SITE 2: note = D-alanine
  MOD_RES 40: note = PolyEthylene Glycol
HAQGTFTSDYAKYLDARRAKEFIAWLVNGGPSSGAPPPSC

SEQ ID NO: 313 - protein, 39 aa, synthetic construct
  SITE 2: note = D-alanine
  MOD_RES 17: note = AminoEthoxyEthoxyAcetic Acyl-AminoEthoxyEthoxyAcetic Acyl-gamma-glutamic acid 1-Amido Eicosanedioic acyl // 2-[(20-oxoicosanoyl)amino]-5 -[2-[2-[2-[2-[2-(carboxymethoxy)ethoxy]ethylamino]-2-oxoet hoxy]ethoxy]ethylamino]-5-oxopentanoic acyl
HAQGTFTSDYAKYLDAKRAKEFIAWLVNGGPSSGAPPPS

SEQ ID NO: 314 - protein, 39 aa, synthetic construct; identical to SEQ ID NO: 313
  SITE 2: note = D-alanine
  MOD_RES 17: note = AminoEthoxyEthoxyAcetic Acyl-AminoEthoxyEthoxyAcetic Acyl-gamma-glutamic acid 1-Amido Eicosanedioic acyl // 2-[(20-oxoicosanoyl)amino]-5 -[2-[2-[2-[2-[2-(carboxymethoxy)ethoxy]ethylamino]-2-oxoet hoxy]ethoxy]ethylamino]-5-oxopentanoic acyl

SEQ ID NO: 315 - protein, 39 aa, synthetic construct; identical to SEQ ID NO: 313
  SITE 2: note = D-alanine
  MOD_RES 17: note = AminoEthoxyEthoxyAcetic Acyl-AminoEthoxyEthoxyAcetic Acyl-gamma-glutamic acid 1-Amido 19-Phosphatidyl Nonadecanoic acyl //

SEQ ID NO: 316 - protein, 39 aa, synthetic construct; identical to SEQ ID NO: 313
  SITE 2: note = D-alanine
  MOD_RES 17: note = N6-[1-O-(17-carboxyheptadecyl)-D-glucopyranuronoyl]

SEQ ID NO: 317 - protein, 39 aa, synthetic construct; identical to SEQ ID NO: 179
  MOD_RES 2: note = Aib
  MOD_RES 20: note = Aib
  MOD_RES 17: note = AminoEthoxyEthoxyAcetic Acyl-AminoEthoxyEthoxyAcetic Acyl-gamma-glutamic acid 1-Amido Eicosanedioic acyl // 2-[(20-oxoicosanoyl)amino]-5 -[2-[2-[2-[2-[2-(carboxymethoxy)ethoxy]ethylamino]-2-oxoet hoxy]ethoxy]ethylamino]-5-oxopentanoic acyl
  MOD_RES 13: note = Alpha-Methyl-Leucine

SEQ ID NO: 318 - protein, 33 aa, synthetic construct
HADGSFSDEMNTILDNLAARDFINWLIQTKITD

SEQ ID NO: 319 - protein, 28 aa, synthetic construct
HSDAVFTDNYTRLRKQMAVKKYLNSILN

SEQ ID NO: 320 - protein, 37 aa, synthetic construct [sequence omitted]

SEQ ID NO: 321 - protein, 29 aa, synthetic construct
YADAIFTNSYRKVLGQLSARKLLQDIMSR

SEQ ID NO: 322 - protein, 40 aa, synthetic construct; identical to SEQ ID NO: 183
  VARIANT 1: note = X can be H, Y or F
  VARIANT 2: note = X can be G, D-Alanine, Aib or D-serine,Amino-cyclobutyl-1-carboxylic acid
  SITE 2: note = D-alanine or D-serine
  VARIANT 3: note = X can be A,R,N,D,E,Q,G,H,I,L,K,M,F,P,S,T,W,Y,D-alanine,Aib,D-serine or D-Lysine
  SITE 3: note = D-alanine,D-serine or D-Lysine
  MOD_RES 2: note = Aib or Amino-cyclobutyl-1-carboxylic acid
  MOD_RES 3: note = Aib
  VARIANT 10: note = X can be A,R,N,D,E,Q,G,H,I,L,K,M,F,P,S,T,W,Y,D-alanine,Aib,D-serine or D-Lysine
  SITE 10: note = D-alanine,D-serine or D-Lysine
  MOD_RES 10: note = Aib
  VARIANT 11: note = X can be A,R,N,D,E,Q,G,H,I,L,K,M,F,P,S,T,W,Y,D-alanine,Aib,D-serine or D-Lysine
  SITE 11: note = D-alanine,D-serine or D-Lysine
  MOD_RES 11: note = Aib
  VARIANT 12: note = X can be A,R,N,D,E,Q,G,H,I,L,K,M,F,P,S,T,W,Y,D-alanine,Aib,D-serine or D-Lysine
  SITE 12: note = D-alanine,D-serine or D-Lysine
  MOD_RES 12: note = Aib
  VARIANT 13: note = X can be Y, Aib, Q or Alpha-Methyl-Leucine
  MOD_RES 13: note = Aib or Alpha-Methyl-Leucine
  VARIANT 15: note = X can be D or E
  VARIANT 16: note = X can be A,R,N,D,E,Q,G,H,I,L,K,M,F,P,S,T,W,Y,D-alanine,Aib,D-serine or D-Lysine
  SITE 16: note = D-alanine,D-serine or D-Lysine
  MOD_RES 16: note = Aib
  VARIANT 17: note = X can be A,R,N,D,E,Q,G,H,I,L,K,M,F,P,S,T,W,Y,D-alanine,Aib,D-serine or D-Lysine
  SITE 17: note = D-alanine,D-serine or D-Lysine
  MOD_RES 17: note = Aib
  VARIANT 18: note = X can be A,R,N,D,E,Q,G,H,I,L,K,M,F,P,S,T,W,Y,D-alanine,Aib,D-serine or D-Lysine
  SITE 18: note = D-alanine,D-serine or D-Lysine
  MOD_RES 18: note = Aib
  VARIANT 19: note = X can be Q or A
  VARIANT 20: note = X can be Q, K ,R or Aib
  MOD_RES 20: note = Aib
  VARIANT 21: note = X can be D, E or A
  VARIANT 23: note = X can be V or I
  VARIANT 24: note = X can be Q, A, E or N
  VARIANT 28: note = X can be A,R,N,D,E,Q,G,H,I,L,K,M,F,P,S,T,W,Y,D-alanine,Aib,D-serine or D-Lysine
  SITE 28: note = D-alanine,D-serine or D-Lysine
  MOD_RES 28: note = Aib
  VARIANT 27: note = X can be A,R,N,D,E,Q,G,H,I,L,K,M,F,P,S,T,W,Y,D-alanine,Aib,D-serine or D-Lysine
  SITE 27: note = D-alanine,D-serine or D-Lysine
  MOD_RES 30: note = Aib
  VARIANT 30: note = X can be A,R,N,D,E,Q,G,H,I,L,K,M,F,P,S,T,W,Y,D-alanine,Aib,D-serine or D-Lysine
  SITE 30: note = D-alanine,D-serine or D-Lysine
  MOD_RES 27: note = Aib
  VARIANT 40: note = X can be K, AminoEthoxyEthoxyAcetic Acid // 8-amino-3,6-dioxa-octanoic acid or absent
  MOD_RES 40: note = AminoEthoxyEthoxyAcetic Acid // 8-amino-3,6-dioxa-octanoic acid

SEQ ID NO: 323 - protein, 39 aa, synthetic construct
  VARIANT 1: note = X can be H, Y or F
  VARIANT 2: note = X can be G, D-Alanine, Aib or D-serine,Amino-cyclobutyl-1-carboxylic acid
  SITE 2: note = D-alanine or D-serine
  MOD_RES 2: note = Aib or Amino-cyclobutyl-1-carboxylic acid
  VARIANT 3: note = X can be A,R,N,D,E,Q,G,H,I,L,K,M,F,P,S,T,W,Y,D-alanine,Aib,D-serine or D-Lysine
  SITE 3: note = D-alanine,D-serine or D-Lysine
  MOD_RES 3: note = Aib
  VARIANT 10: note = X can be A,R,N,D,E,Q,G,H,I,L,K,M,F,P,S,T,W,Y,D-alanine,Aib,D-serine or D-Lysine
  SITE 10: note = D-alanine,D-serine or D-Lysine
  MOD_RES 10: note = Aib
  VARIANT 11: note = X can be A,R,N,D,E,Q,G,H,I,L,K,M,F,P,S,T,W,Y,D-alanine,Aib,D-serine or D-Lysine
  SITE 11: note = D-alanine,D-serine or D-Lysine
  MOD_RES 11: note = Aib
  VARIANT 12: note = X can be A,R,N,D,E,Q,G,H,I,L,K,M,F,P,S,T,W,Y,D-alanine,Aib,D-serine or D-Lysine
  SITE 12: note = D-alanine,D-serine or D-Lysine
  MOD_RES 12: note = Aib
  VARIANT 13: note = X can be Y, Aib, Q or Alpha-Methyl-Leucine
  MOD_RES 13: note = Aib or Alpha-Methyl-Leucine
  VARIANT 15: note = X can be D or E
  VARIANT 16: note = X can be A,R,N,D,E,Q,G,H,I,L,K,M,F,P,S,T,W,Y,D-alanine,Aib,D-serine or D-Lysine
  SITE 16: note = D-alanine,D-serine or D-Lysine
  MOD_RES 2: note = Aib
  MOD_RES 16: note = Aib
  VARIANT 17: note = X can be A,R,N,D,E,Q,G,H,I,L,K,M,F,P,S,T,W,Y,D-alanine,Aib,D-serine or D-Lysine
  SITE 17: note = D-alanine,D-serine or D-Lysine
  MOD_RES 17: note = Aib
  VARIANT 18: note = X can be A,R,N,D,E,Q,G,H,I,L,K,M,F,P,S,T,W,Y,D-alanine,Aib,D-serine or D-Lysine
  SITE 18: note = D-alanine,D-serine or D-Lysine
  MOD_RES 18: note = Aib
  VARIANT 19: note = X can be Q or A
  VARIANT 20: note = X can be Q, K ,R or Aib
  MOD_RES 20: note = Aib
  VARIANT 21: note = X can be D, E or A
  VARIANT 23: note = X can be V or I
  VARIANT 24: note = X can be Q, A, E or N
  VARIANT 28: note = X can be A,R,N,D,E,Q,G,H,I,L,K,M,F,P,S,T,W,Y,D-alanine,Aib,D-serine or D-Lysine
  SITE 28: note = D-alanine,D-serine or D-Lysine
  MOD_RES 28: note = Aib
  VARIANT 27: note = X can be A,R,N,D,E,Q,G,H,I,L,K,M,F,P,S,T,W,Y,D-alanine,Aib,D-serine or D-Lysine
  MOD_RES 30: note = Aib
  VARIANT 30: note = X can be A,R,N,D,E,Q,G,H,I,L,K,M,F,P,S,T,W,Y,D-alanine,Aib,D-serine or D-Lysine
  SITE 30: note = D-alanine,D-serine or D-Lysine
  MOD_RES 27: note = Aib
XXXGTFTSDXXXXLXXXXXXXFXXWLXXGXPSSGAPPPS

SEQ ID NO: 324 - protein, 11 aa, synthetic construct
  VARIANT 1: note = X can be A,R,N,D,E,Q,G,H,I,L,K,M,F,P,S,T,W,Y,D-alanine,Aib,D-serine or D-Lysine
  SITE 1: note = D-alanine,D-serine or D-Lysine
  MOD_RES 1: note = Aib
  VARIANT 11: note = X can be K, AminoEthoxyEthoxyAcetic Acid // 8-amino-3,6-dioxa-octanoic acid or absent
  MOD_RES 11: note = AminoEthoxyEthoxyAcetic Acid // 8-amino-3,6-dioxa-octanoic acid
XPSSGAPPPSX

SEQ ID NO: 325 - protein, 12 aa, synthetic construct
  VARIANT 1: note = X can be A,R,N,D,E,Q,G,H,I,L,K,M,F,P,S,T,W,Y,D-alanine,Aib,D-serine or D-Lysine
  SITE 1: note = D-alanine,D-serine or D-Lysine
  MOD_RES 1: note = Aib
  VARIANT 2: note = X can be Q or A
  VARIANT 3: note = X can be Q, K ,R or Aib
  MOD_RES 3: note = Aib
  VARIANT 4: note = X can be D, E or A
  VARIANT 6: note = X can be V or I
  VARIANT 7: note = X can be Q, A, E or N
  VARIANT 10: note = X can be A,R,N,D,E,Q,G,H,I,L,K,M,F,P,S,T,W,Y,D-alanine,Aib,D-serine or D-Lysine
  SITE 10: note = D-alanine,D-serine or D-Lysine
  MOD_RES 10: note = Aib
  VARIANT 11: note = X can be A,R,N,D,E,Q,G,H,I,L,K,M,F,P,S,T,W,Y,D-alanine,Aib,D-serine or D-Lysine
  SITE 11: note = D-alanine,D-serine or D-Lysine
  MOD_RES 11: note = Aib
XXXXFXXWLXXG

SEQ ID NO: 326 - protein, 17 aa, synthetic construct
  VARIANT 1: note = X can be H, Y or F
  VARIANT 2: note = X can be G, D-Alanine, Aib or D-serine,Amino-cyclobutyl-1-carboxylic acid
  SITE 2: note = D-alanine or D-serine
  MOD_RES 2: note = Aib or Amino-cyclobutyl-1-carboxylic acid
  VARIANT 3: note = X can be A,R,N,D,E,Q,G,H,I,L,K,M,F,P,S,T,W,Y,D-alanine,Aib,D-serine or D-Lysine
  SITE 3: note = D-alanine,D-serine or D-Lysine
  MOD_RES 3: note = Aib
  VARIANT 10: note = X can be A,R,N,D,E,Q,G,H,I,L,K,M,F,P,S,T,W,Y,D-alanine,Aib,D-serine or D-Lysine
  SITE 10: note = D-alanine,D-serine or D-Lysine
  MOD_RES 10: note = Aib
  VARIANT 11: note = X can be A,R,N,D,E,Q,G,H,I,L,K,M,F,P,S,T,W,Y,D-alanine,Aib,D-serine or D-Lysine
  SITE 11: note = D-alanine,D-serine or D-Lysine
  MOD_RES 11: note = Aib
  VARIANT 12: note = X can be A,R,N,D,E,Q,G,H,I,L,K,M,F,P,S,T,W,Y,D-alanine,Aib,D-serine or D-Lysine
  SITE 12: note = D-alanine,D-serine or D-Lysine
  MOD_RES 12: note = Aib
  VARIANT 13: note = X can be Y, Aib, Q or Alpha-Methyl-Leucine
  MOD_RES 13: note = Aib or Alpha-Methyl-Leucine
  VARIANT 15: note = X can be D or E
  VARIANT 16: note = X can be A,R,N,D,E,Q,G,H,I,L,K,M,F,P,S,T,W,Y,D-alanine,Aib,D-serine or D-Lysine
  SITE 16: note = D-alanine,D-serine or D-Lysine
  MOD_RES 16: note = Aib
  VARIANT 17: note = X can be A,R,N,D,E,Q,G,H,I,L,K,M,F,P,S,T,W,Y,D-alanine,Aib,D-serine or D-Lysine
  SITE 17: note = D-alanine,D-serine or D-Lysine
  MOD_RES 17: note = Aib
XXXGTFTSDXXXXLXXX

SEQ ID NO: 327 - protein, 11 aa, synthetic construct
  VARIANT 1: note = X can be A,R,N,D,E,Q,G,H,I,L,K,M,F,P,S,T,W,Y,D-alanine,Aib,D-serine or D-Lysine
  SITE 1: note = D-alanine,D-serine or D-Lysine
  MOD_RES 1: note = Aib
XPSSGAPPPSK

SEQ ID NO: 328 - protein, 23 aa, synthetic construct
  VARIANT 1: note = X can be A,R,N,D,E,Q,G,H,I,L,K,M,F,P,S,T,W,Y,D-alanine,Aib,D-serine or D-Lysine
  SITE 1: note = D-alanine,D-serine or D-Lysine
  MOD_RES 1: note = Aib
  VARIANT 2: note = X can be Q or A
  VARIANT 3: note = X can be Q, K ,R or Aib
  MOD_RES 3: note = Aib
  VARIANT 21: note = X can be D, E or A
  VARIANT 6: note = X can be V or I
  VARIANT 7: note = X can be Q, A, E or N
  VARIANT 10: note = X can be A,R,N,D,E,Q,G,H,I,L,K,M,F,P,S,T,W,Y,D-alanine,Aib,D-serine or D-Lysine
  SITE 10: note = D-alanine,D-serine or D-Lysine
  MOD_RES 10: note = Aib
  VARIANT 11: note = X can be A,R,N,D,E,Q,G,H,I,L,K,M,F,P,S,T,W,Y,D-alanine,Aib,D-serine or D-Lysine
  SITE 11: note = D-alanine,D-serine or D-Lysine
  MOD_RES 11: note = Aib
  VARIANT 13: note = X can be A,R,N,D,E,Q,G,H,I,L,K,M,F,P,S,T,W,Y,D-alanine,Aib,D-serine or D-Lysine
  SITE 13: note = D-alanine,D-serine or D-Lysine
  MOD_RES 13: note = Aib
  VARIANT 23: note = X can be K, AminoEthoxyEthoxyAcetic Acid // 8-amino-3,6-dioxa-octanoic acid or absent
  MOD_RES 23: note = AminoEthoxyEthoxyAcetic Acid // 8-amino-3,6-dioxa-octanoic acid
  VARIANT 4: note = X can be D, E or A
XXXXFXXWLXXGXPSSGAPPPSX

SEQ ID NO: 329 - protein, 40 aa, synthetic construct; identical to SEQ ID NO: 183
  VARIANT 1: note = X can be H, Y or F
  VARIANT 2: note = X can be G, D-Alanine, Aib or D-serine,Amino-cyclobutyl-1-carboxylic acid
  SITE 2: note = D-alanine or D-serine
  VARIANT 3: note = X can be A,R,N,D,E,Q,G,H,I,L,K,M,F,P,S,T,W,Y,D-alanine,Aib,D-serine or D-Lysine
  SITE 3: note = D-alanine,D-serine or D-Lysine
  MOD_RES 2: note = Aib or Amino-cyclobutyl-1-carboxylic acid
  MOD_RES 3: note = Aib
  VARIANT 10: note = X can be A,R,N,D,E,Q,G,H,I,L,K,M,F,P,S,T,W,Y,D-alanine,Aib,D-serine or D-Lysine
  SITE 10: note = D-alanine,D-serine or D-Lysine
  MOD_RES 10: note = Aib
  VARIANT 11: note = X can be A,R,N,D,E,Q,G,H,I,L,K,M,F,P,S,T,W,Y,D-alanine,Aib,D-serine or D-Lysine
  SITE 11: note = D-alanine,D-serine or D-Lysine
  MOD_RES 11: note = Aib
  VARIANT 12: note = X can be A,R,N,D,E,Q,G,H,I,L,K,M,F,P,S,T,W,Y,D-alanine,Aib,D-serine or D-Lysine
  SITE 12: note = D-alanine,D-serine or D-Lysine
  MOD_RES 12: note = Aib
  VARIANT 13: note = X can be Y, Aib, Q or Alpha-Methyl-Leucine
  MOD_RES 13: note = Aib or Alpha-Methyl-Leucine
  VARIANT 15: note = X can be D or E
  VARIANT 16: note = X can be A,R,N,D,E,Q,G,H,I,L,K,M,F,P,S,T,W,Y,D-alanine,Aib,D-serine or D-Lysine
  SITE 16: note = D-alanine,D-serine or D-Lysine
  MOD_RES 16: note = Aib
  VARIANT 17: note = X can be A,R,N,D,E,Q,G,H,I,L,K,M,F,P,S,T,W,Y,D-alanine,Aib,D-serine or D-Lysine
  SITE 17: note = D-alanine,D-serine or D-Lysine
  MOD_RES 17: note = Aib
  VARIANT 18: note = X can be A,R,N,D,E,Q,G,H,I,L,K,M,F,P,S,T,W,Y,D-alanine,Aib,D-serine or D-Lysin
  SITE 18: note = D-alanine,D-serine or D-Lysine
  MOD_RES 18: note = Aib
  VARIANT 19: note = X can be Q or A
  VARIANT 20: note = X can be Q, K ,R or Aib
  MOD_RES 20: note = Aib
  VARIANT 21: note = X can be D, E or A
  VARIANT 23: note = X can be V or I
  VARIANT 24: note = X can be Q, A, E or N
  VARIANT 28: note = X can be A,R,N,D,E,Q,G,H,I,L,K,M,F,P,S,T,W,Y,D-alanine,Aib,D-serine or D-Lysine
  SITE 28: note = D-alanine,D-serine or D-Lysine
  MOD_RES 28: note = Aib
  VARIANT 27: note = X can be A,R,N,D,E,Q,G,H,I,L,K,M,F,P,S,T,W,Y,D-alanine,Aib,D-serine or D-Lysine
  SITE 27: note = D-alanine,D-serine or D-Lysine
  MOD_RES 30: note = Aib
  VARIANT 30: note = X can be A,R,N,D,E,Q,G,H,I,L,K,M,F,P,S,T,W,Y,D-alanine,Aib,D-serine or D-Lysine
  SITE 30: note = D-alanine,D-serine or D-Lysine
  MOD_RES 27: note = Aib
  VARIANT 40: note = X can be K, AminoEthoxyEthoxyAcetic Acid // 8-amino-3,6-dioxa-octanoic acid or absent
  MOD_RES 40: note = AminoEthoxyEthoxyAcetic Acid // 8-amino-3,6-dioxa-octanoic acid

SEQ ID NO: 330 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDVSSYLEGQAAKEFIAWLVKGRPSSGAPPPSK

SEQ ID NO: 331 - protein, 40 aa, synthetic construct; identical to SEQ ID NO: 330
  MOD_RES 40: note = AminoEthoxyEthoxyAcetic Acyl-MaleimidoPropionic Acyl

SEQ ID NO: 332 - protein, 31 aa, synthetic construct
  SITE 2: note = D-alanine
  MOD_RES 31: note = AminoEthoxyEthoxyAcetic Acyl-MaleimidoPropionic Acyl
HAEGTFTSDVSSYLEGQAAKEFIAWLVNGGK

SEQ ID NO: 333 - protein, 31 aa, synthetic construct
  SITE 2: note = D-alanine
  MOD_RES 31: note = AminoEthoxyEthoxyAcetic Acyl-MaleimidoPropionic Acyl
HAEGTFTSDINKVLDTIAAKEFIAWLVKGRK

SEQ ID NO: 334 - protein, 31 aa, synthetic construct
  SITE 2: note = D-alanine
  MOD_RES 31: note = AminoEthoxyEthoxyAcetic Acyl-MaleimidoPropionic Acyl
HAEGTFTSDAEKAKEAEKAKEFIAWLVKGRK

SEQ ID NO: 335 - protein, 31 aa, synthetic construct
  SITE 2: note = D-alanine
  MOD_RES 31: note = AminoEthoxyEthoxyAcetic Acyl-MaleimidoPropionic Acyl
  MOD_RES 10: note = Aib
  MOD_RES 13: note = Aib
  MOD_RES 16: note = Aib
HAEGTFTSDXEKXKEXEKAKEFIAWLVKGRK

SEQ ID NO: 336 - protein, 40 aa, synthetic construct
  SITE 2: note = D-alanine
  MOD_RES 10: note = Aib
  MOD_RES 13: note = Aib
  MOD_RES 16: note = Aib
  MOD_RES 19: note = Aib
  MOD_RES 40: note = AminoEthoxyEthoxyAcetic Acyl-MaleimidoPropionic Acyl
HAEGTFTSDXESXKEXEKXKEFIAWLVKGRPSSGAPPPSK

SEQ ID NO: 337 - protein, 40 aa, synthetic construct
  SITE 2: note = D-alanine
  MOD_RES 11: note = Aib
  MOD_RES 14: note = Aib
  MOD_RES 18: note = Aib
  MOD_RES 40: note = AminoEthoxyEthoxyAcetic Acyl-MaleimidoPropionic Acyl
HAEGTFTSDVXSYXEGQXAKEFIAWLVKGRPSSGAPPPSK

SEQ ID NO: 338 - protein, 39 aa, synthetic construct
  SITE 2: note = D-alanine
  MOD_RES 17: note = AminoEthoxyEthoxyAcetic Acyl-MaleimidoPropionic Acyl
HAEGTFTSDVSSYLEGKAAKEFIAWLVKGRPSSGAPPPS

SEQ ID NO: 339 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDVSSYLEGQAAKEFIAWLVKGRLLLLLLLLLK

SEQ ID NO: 340 - protein, 42 aa, synthetic construct
  SITE 2: note = D-alanine
HAEGTFTSDVSSYLEGQAAKEFIAWLVKGRPEAPTDPEAPTD

SEQ ID NO: 341 - protein, 40 aa, synthetic construct
  MOD_RES 40: note = AminoEthoxyEthoxyAcetic Acyl-MaleimidoPropionic Acyl
HGEGTFTSDLSKQMEEEAVRLFIEWLKNGGGGGGGGGGGK

SEQ ID NO: 342 - protein, 31 aa, synthetic construct; identical to SEQ ID NO: 1
  SITE 2: note = D-alanine
  MOD_RES 31: note = (AminoEthoxyEthoxyAcetic Acyl)4-3-MaleimidoPropionic Acyl

SEQ ID NO: 343 - protein, 30 aa, synthetic construct
  SITE 2: note = D-alanine
  MOD_RES 17: note = AminoEthoxyEthoxyAcetic Acyl-MaleimidoPropionic Acyl
HAEGTFTSDVSSYLEGKAAKEFIAWLVKGR

SEQ ID NO: 344 - protein, 31 aa, synthetic construct; identical to SEQ ID NO: 1
  SITE 2: note = D-alanine
  MOD_RES 31: note = 3-MaleimidoPropionic Acid // 3-(2,5-Dioxo-2,5-dihydro-1H-pyrrol-1-yl)propanoic Acid

SEQ ID NO: 345 - protein, 37 aa, synthetic construct
HSQGTFTSDYSKYLDSRRAQDFVQWLMNTKRNKNNIA

SEQ ID NO: 346 - protein, 41 aa, synthetic construct
  SITE 2: note = D-alanine
HAEGTFTSDVSSYLEGQAAKEFIAWLVKGPEAPTDPEAPTD

SEQ ID NO: 347 - protein, 31 aa, synthetic construct; identical to SEQ ID NO: 1
  SITE 2: note = D-alanine
  MOD_RES 31: note = (AminoEthoxyEthoxyAcetic Acyl)4-3-MaleimidoPropionic Acyl

SEQ ID NO: 348 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDINKVLDIIAAKEFIAWLVKGRPSSGAPPPSK

SEQ ID NO: 349 - protein, 31 aa, synthetic construct
  SITE 2: note = D-alanine
  MOD_RES 31: note = AminoEthoxyEthoxyAcetic Acyl-MaleimidoPropionic Acyl
HAEGTFTSDINKVLDIIAAKEFIAWLVKGRK

SEQ ID NO: 350 - protein, 40 aa, synthetic construct; identical to SEQ ID NO: 2
  MOD_RES 40: note = 3-MaleimidoPropionic Acid // 3-(2,5-Dioxo-2,5-dihydro-1H-pyrrol-1-yl)propanoic Acid

SEQ ID NO: 351 - protein, 40 aa, synthetic construct
  MOD_RES 2: note = Aib
  MOD_RES 40: note = AminoEthoxyEthoxyAcetic Acyl-MaleimidoPropionic Acyl
HXEGTFTSDYSKYLDKIRAQEFVAWLMNGGPSSGAPPPSK

SEQ ID NO: 352 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDKIAAQDFVAWLLNGGPSSGAPPPSK

SEQ ID NO: 353 - protein, 40 aa, synthetic construct
  MOD_RES 40: note = AminoEthoxyEthoxyAcetic Acyl-MaleimidoPropionic Acyl
  MOD_RES 2: note = Aib
HXQGTFTSDYAKYLDKIAAQDFVAWLLDGGPSSGAPPPSK

SEQ ID NO: 354 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDKIAAQDFVAWLLDGGPSSGAPPPSK

SEQ ID NO: 355 - protein, 40 aa, synthetic construct
  MOD_RES 2: note = Aib
  MOD_RES 40: note = AminoEthoxyEthoxyAcetic Acyl-AminoEthoxyEthoxyAcetic Acyl-MaleimidoPropionic Acyl
HXQGTFTSDYSKYLDKIAAQDFVAYLLDGGPSSGAPPPSK

SEQ ID NO: 356 - protein, 40 aa, synthetic construct
  MOD_RES 40: note = AminoEthoxyEthoxyAcetic Acyl-AminoEthoxyEthoxyAcetic Acyl-MaleimidoPropionic Acyl
  MOD_RES 2: note = Aib
HXQGTFTSDLSKYLDEIAVQDFIEWLLDGGPSSGAPPPSK

SEQ ID NO: 357 - protein, 37 aa, synthetic construct
HSQGTFTSDYSKYLDSRRAQDFVQWLMNTKRNRNNIA

SEQ ID NO: 358 - protein, 34 aa, synthetic construct
IKPEAPGEDASPEELNRYYASLRHYLNLVTRQRY

SEQ ID NO: 359 - protein, 31 aa, synthetic construct
  MOD_RES 2: note = Aib
HXEGTFTSDVSSYLEGQALRHYINWLTRQRY

SEQ ID NO: 360 - protein, 45 aa, synthetic construct
HGEGTFTSDLSKQMEEEAVRLFIEWLKNGGPSSRHYLNLVTRQRY

SEQ ID NO: 361 - protein, 51 aa, synthetic construct
  SITE 2: note = D-Serine
HXQGTFTSDLSKYLEEEAVREFIAWLKNGGPSSGAPPPSRHYLNLVTRQRY

SEQ ID NO: 362 - protein, 39 aa, synthetic construct
  SITE 2: note = D-Serine
HXQGTYTNDVSKYXDSRRAQDFIEWLKNGGPSSGAPPPS

SEQ ID NO: 363 - protein, 40 aa, synthetic construct
  SITE 2: note = D-Serine
HXQGTYTNDVSKYKDSRRAQDFIEWLKNGGPSSGAPPPSC

SEQ ID NO: 364 - protein, 40 aa, synthetic construct
  SITE 2: note = D-Serine
HXQGTFTSDLSKQKDSRRAQDFIEWLKNGGPSSGAPPPSC

SEQ ID NO: 365 - protein, 40 aa, synthetic construct
  SITE 2: note = D-Serine
HXQGTYTNDVSKYXDSRRAQDFIEWLKNGGPSSGAPPPSC

SEQ ID NO: 366 - protein, 34 aa, synthetic construct
PKPEAPGKDASPEEWNRYYADLRHYLNWLTRQRY

SEQ ID NO: 367 - protein, 34 aa, synthetic construct
IKPEAPGEDASPEELNRYYASLRHYLNWVTRQXY

SEQ ID NO: 368 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
YXEGTFTSDYSIYLDKQAAXAFVQWLIAGGPSSGAPPPSK

SEQ ID NO: 369 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
YXEGTFTSDYSIYLDKQAAXAFVQWLLAGGPSSGAPPPSK

SEQ ID NO: 370 - protein, 40 aa, synthetic construct
  MOD_RES 40: note = AminoEthoxyEthoxyAcetic Acyl-MaleimidoPropionic Acyl
  MOD_RES 2: note = Aib
  MOD_RES 20: note = Aib
YXEGTFTSDYSIYLDKQAAXEFVQWLIAGGPSSGAPPPSK

SEQ ID NO: 371 - protein, 40 aa, synthetic construct
  SITE 2: note = D-alanine
  MOD_RES 40: note = AminoEthoxyEthoxyAcetic Acyl-MaleimidoPropionic Acyl
  MOD_RES 20: note = Aib
HAEGTFTSDYSIYLDKQAAXEFVQWLLAGGPSSGAPPPSK

SEQ ID NO: 372 - protein, 40 aa, synthetic construct
  MOD_RES 2: note = D-alanine
  MOD_RES 40: note = AminoEthoxyEthoxyAcetic Acyl-MaleimidoPropionic Acyl
  MOD_RES 20: note = Aib
YAEGTFTSDYSIYLDKQAAXEFVQWLLAGGPSSGAPPPSK

SEQ ID NO: 373 - protein, 40 aa, synthetic construct
  MOD_RES 2: note = Aib
  MOD_RES 20: note = Aib
  MOD_RES 40: note = AminoEthoxyEthoxyAcetic Acyl-MaleimidoPropionic Acyl
HXEGTFTSDYSIYLDKQAAXEFVQWLLAGGPSSGAPPPSK

SEQ ID NO: 374 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
HAEGTFTSDYSIYLDKQAAXAFVQWLIAGGPSSGAPPPSK

SEQ ID NO: 375 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
HAEGTFTSDYSIYLDKQAAXAFVQWLLAGGPSSGAPPPSK

SEQ ID NO: 376 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
HAEGTFTSDYSIYLDKQAAXEFVQWLIAGGPSSGAPPPSK

SEQ ID NO: 377 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
YAEGTFTSDYSIYLDKQAAXAFVQWLIAGGPSSGAPPPSK

SEQ ID NO: 378 - protein, 40 aa, synthetic construct
  SITE 2: note = D-alanine
  MOD_RES 20: note = Aib
  MOD_RES 40: note = AminoEthoxyEthoxyAcetic Acyl-MaleimidoPropionic Acyl
YAEGTFTSDYSIYLDKQAAXAFVQWLLAGGPSSGAPPPSK

SEQ ID NO: 379 - protein, 40 aa, synthetic construct
  MOD_RES 20: note = Aib
  SITE 2: note = D-alanine
  MOD_RES 40: note = AminoEthoxyEthoxyAcetic Acyl-MaleimidoPropionic Acyl
YAEGTFTSDYSIYLDKQAAXEFVQWLIAGGPSSGAPPPSK

SEQ ID NO: 380 - protein, 40 aa, synthetic construct
  MOD_RES 2: note = Aib
  MOD_RES 40: note = AminoEthoxyEthoxyAcetic Acyl-MaleimidoPropionic Acyl
YXEGTFTSDYAKYLDARRAKEFVQWLLAGGPSSGAPPPSK

SEQ ID NO: 381 - protein, 40 aa, synthetic construct
  MOD_RES 2: note = Aib
  MOD_RES 40: note = AminoEthoxyEthoxyAcetic Acyl-MaleimidoPropionic Acyl
YXEGTFTSDYSKYLDARRAKEFVQWLLAGGPSSGAPPPSK

SEQ ID NO: 382 - protein, 40 aa, synthetic construct
  MOD_RES 2: note = Aib
  MOD_RES 40: note = AminoEthoxyEthoxyAcetic Acyl-MaleimidoPropionic Acyl
YXEGTFTSDYSKYLDKRRAKEFVQWLLAGGPSSGAPPPSK

SEQ ID NO: 383 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFVQWLLAGGPSSGAPPPSK

SEQ ID NO: 384 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYSKYLDARRAKEFVQWLLAGGPSSGAPPPSK

SEQ ID NO: 385 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYSKYLDKRRAKEFVQWLLAGGPSSGAPPPSK

SEQ ID NO: 386 - protein, 40 aa, synthetic construct
  SITE 2: note = D-alanine
  MOD_RES 40: note = AminoEthoxyEthoxyAcetic Acyl-MaleimidoPropionic Acyl
YAEGTFTSDYAKYLDARRAKEFVQWLLAGGPSSGAPPPSK

SEQ ID NO: 387 - protein, 40 aa, synthetic construct
  SITE 2: note = D-alanine
  MOD_RES 40: note = AminoEthoxyEthoxyAcetic Acyl-MaleimidoPropionic Acyl
YAEGTFTSDYSKYLDARRAKEFVQWLLAGGPSSGAPPPSK

SEQ ID NO: 388 - protein, 40 aa, synthetic construct
  SITE 2: note = D-alanine
  MOD_RES 40: note = AminoEthoxyEthoxyAcetic Acyl-MaleimidoPropionic Acyl
YAEGTFTSDYSKYLDKRRAKEFVQWLLAGGPSSGAPPPSK

SEQ ID NO: 389 - protein, 40 aa, synthetic construct
  MOD_RES 40: note = AminoEthoxyEthoxyAcetic Acyl-MaleimidoPropionic Acyl
  MOD_RES 2: note = Aib
HXEGTFTSDYAKYLDARRAKEFVQWLLAGGPSSGAPPPSK

SEQ ID NO: 390 - protein, 40 aa, synthetic construct
  MOD_RES 2: note = Aib
  MOD_RES 40: note = AminoEthoxyEthoxyAcetic Acyl-MaleimidoPropionic Acyl
HXEGTFTSDYSKYLDARRAKEFVQWLLAGGPSSGAPPPSK

SEQ ID NO: 391 - protein, 40 aa, synthetic construct
  MOD_RES 2: note = Aib
  MOD_RES 40: note = AminoEthoxyEthoxyAcetic Acyl-MaleimidoPropionic Acyl
HXEGTFTSDYSKYLDKRRAKEFVQWLLAGGPSSGAPPPSK

SEQ ID NO: 392 - protein, 40 aa, synthetic construct
  MOD_RES 2: note = Aib
  MOD_RES 40: note = AminoEthoxyEthoxyAcetic Acyl-MaleimidoPropionic Acyl
YXEGTFTSDYAKYLDARRAKAFVQWLIAGGPSSGAPPPSK

SEQ ID NO: 393 - protein, 40 aa, synthetic construct
  MOD_RES 2: note = Aib
  MOD_RES 40: note = AminoEthoxyEthoxyAcetic Acyl-MaleimidoPropionic Acyl
YXEGTFTSDYSKYLDARRAKAFVQWLIAGGPSSGAPPPSK

SEQ ID NO: 394 - protein, 40 aa, synthetic construct
  MOD_RES 2: note = Aib
  MOD_RES 40: note = AminoEthoxyEthoxyAcetic Acyl-MaleimidoPropionic Acyl
YXEGTFTSDYSKYLDKRRAKAFVQWLIAGGPSSGAPPPSK

SEQ ID NO: 395 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKAFVQWLIAGGPSSGAPPPSK

SEQ ID NO: 396 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYSKYLDARRAKAFVQWLIAGGPSSGAPPPSK

SEQ ID NO: 397 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYSKYLDKRRAKAFVQWLIAGGPSSGAPPPSK

SEQ ID NO: 398 - protein, 40 aa, synthetic construct
  SITE 2: note = D-alanine
  MOD_RES 40: note = AminoEthoxyEthoxyAcetic Acyl-MaleimidoPropionic Acyl
YAEGTFTSDYAKYLDARRAKAFVQWLIAGGPSSGAPPPSK

SEQ ID NO: 399 - protein, 40 aa, synthetic construct
  SITE 2: note = D-alanine
  MOD_RES 40: note = AminoEthoxyEthoxyAcetic Acyl-MaleimidoPropionic Acyl
YAEGTFTSDYSKYLDARRAKAFVQWLIAGGPSSGAPPPSK

SEQ ID NO: 400 - protein, 40 aa, synthetic construct
  SITE 2: note = D-alanine
  MOD_RES 40: note = AminoEthoxyEthoxyAcetic Acyl-MaleimidoPropionic Acyl
YAEGTFTSDYSKYLDKRRAKAFVQWLIAGGPSSGAPPPSK

SEQ ID NO: 401 - protein, 40 aa, synthetic construct
  MOD_RES 2: note = Aib
  MOD_RES 40: note = AminoEthoxyEthoxyAcetic Acyl-MaleimidoPropionic Acyl
HXEGTFTSDYAKYLDARRAKAFVQWLIAGGPSSGAPPPSK

SEQ ID NO: 402 - protein, 40 aa, synthetic construct
  MOD_RES 2: note = Aib
  MOD_RES 40: note = AminoEthoxyEthoxyAcetic Acyl-MaleimidoPropionic Acyl
HXEGTFTSDYSKYLDARRAKAFVQWLIAGGPSSGAPPPSK

SEQ ID NO: 403 - protein, 40 aa, synthetic construct
  MOD_RES 2: note = Aib
  MOD_RES 40: note = AminoEthoxyEthoxyAcetic Acyl-MaleimidoPropionic Acyl
HXEGTFTSDYSKYLDKRRAKAFVQWLIAGGPSSGAPPPSK

SEQ ID NO: 404 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDLSKQLESKAAQDFIEWLKAGGPSSGAPPPSK

SEQ ID NO: 405 - protein, 40 aa, synthetic construct
  SITE 2: note = D-serine
  MOD_RES 40: note = AminoEthoxyEthoxyAcetic Acyl-MaleimidoPropionic Acyl
HSQGTFTSDYAKYLDARRAKEFIEWLKAGGPSSGAPPPSK

SEQ ID NO: 406 - protein, 40 aa, synthetic construct
  SITE 2: note = D-serine
  MOD_RES 40: note = AminoEthoxyEthoxyAcetic Acyl-MaleimidoPropionic Acyl
HSQGTFTSDYSKYLDARRAKEFIEWLKAGGPSSGAPPPSK

SEQ ID NO: 407 - protein, 40 aa, synthetic construct
  SITE 2: note = D-serine
  MOD_RES 40: note = AminoEthoxyEthoxyAcetic Acyl-MaleimidoPropionic Acyl
HSQGTFTSDYAKYLDSRRAKEFIEWLKAGGPSSGAPPPSK

SEQ ID NO: 408 - protein, 40 aa, synthetic construct
  SITE 2: note = D-serine
  MOD_RES 40: note = AminoEthoxyEthoxyAcetic Acyl-MaleimidoPropionic Acyl
HSQGTFTSDYSKYLDSRRAKEFIEWLKAGGPSSGAPPPSK

SEQ ID NO: 409 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDSRRAKEFIEWLKAGGPSSGAPPPSK

SEQ ID NO: 410 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFIEWLKAGGPSSGAPPPSK

SEQ ID NO: 411 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDLSKQLESKAAQDFIAWLVNGGPSSGAPPPSK

SEQ ID NO: 412 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKDFIEWLKAGGPSSGAPPPSK

SEQ ID NO: 413 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAQEFIEWLKAGGPSSGAPPPSK

SEQ ID NO: 414 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDSRAAKEFIEWLKAGGPSSGAPPPSK

SEQ ID NO: 415 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDSKRAKEFIEWLKAGGPSSGAPPPSK

SEQ ID NO: 416 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLEARRAKEFIEWLKAGGPSSGAPPPSK

SEQ ID NO: 417 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKQLEARRAKEFIEWLKAGGPSSGAPPPSK

SEQ ID NO: 418 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDERAAQAFIEYLLEGGPSSGAPPPSK

SEQ ID NO: 419 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDERAAQDFVQWLLDGGPSSGAPPPSK

SEQ ID NO: 420 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDERAAQAFIEYLLEGGPSSGAPPPSK

SEQ ID NO: 421 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDARRAKEFVQWLLDGGPSSGAPPPSK

SEQ ID NO: 422 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDARRAKEFIEYLLEGGPSSGAPPPSK

SEQ ID NO: 423 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYAKYLDARRAKEFVQWLLDGGPSSGAPPPSK

SEQ ID NO: 424 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAKEFVQWLLDGGPSSGAPPPSK

SEQ ID NO: 425 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFVQWLLDGGPSSGAPPPSK

SEQ ID NO: 426 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAKEFIEYLLEGGPSSGAPPPSK

SEQ ID NO: 427 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDERAAQAFIEYLLDGGPSSGAPPPSK

SEQ ID NO: 428 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDERAAQDFVQWLLEGGPSSGAPPPSK

SEQ ID NO: 429 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDERAAQAFIEYLLDGGPSSGAPPPSK

SEQ ID NO: 430 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDARRAKEFVQWLLEGGPSSGAPPPSK

SEQ ID NO: 431 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDARRAKEFIEYLLDGGPSSGAPPPSK

SEQ ID NO: 432 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYAKYLDARRAKEFVQWLLEGGPSSGAPPPSK

SEQ ID NO: 433 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYAKYLDARRAKEFIEYLLDGGPSSGAPPPSK

SEQ ID NO: 434 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAKEFVQWLLEGGPSSGAPPPSK

SEQ ID NO: 435 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFVQWLLEGGPSSGAPPPSK

SEQ ID NO: 436 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAKEFIEYLLDGGPSSGAPPPSK

SEQ ID NO: 437 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFIEYLLDGGPSSGAPPPSK

SEQ ID NO: 438 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDERAAQAFIEWLLEGGPSSGAPPPSK

SEQ ID NO: 439 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDERAAQDFVQYLLDGGPSSGAPPPSK

SEQ ID NO: 440 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDERAAQAFIEWLLEGGPSSGAPPPSK

SEQ ID NO: 441 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDARRAKEFVQYLLDGGPSSGAPPPSK

SEQ ID NO: 442 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDARRAKEFIEWLLEGGPSSGAPPPSK

SEQ ID NO: 443 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYAKYLDARRAKEFVQYLLDGGPSSGAPPPSK

SEQ ID NO: 444 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYAKYLDARRAKEFIEWLLEGGPSSGAPPPSK

SEQ ID NO: 445 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAKEFVQYLLDGGPSSGAPPPSK

SEQ ID NO: 446 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFVQYLLDGGPSSGAPPPSK

SEQ ID NO: 447 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAKEFIEWLLEGGPSSGAPPPSK

SEQ ID NO: 448 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFIEWLLEGGPSSGAPPPSK

SEQ ID NO: 449 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDERAAQAFVQYLLEGGPSSGAPPPSK

SEQ ID NO: 450 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDERAAQDFIEWLLDGGPSSGAPPPSK

SEQ ID NO: 451 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDERAAQAFVQYLLEGGPSSGAPPPSK

SEQ ID NO: 452 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDARRAKEFIEWLLDGGPSSGAPPPSK

SEQ ID NO: 453 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDARRAKEFVQYLLEGGPSSGAPPPSK

SEQ ID NO: 454 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYAKYLDARRAKEFIEWLLDGGPSSGAPPPSK

SEQ ID NO: 455 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYAKYLDARRAKEFVQYLLEGGPSSGAPPPSK

SEQ ID NO: 456 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAKEFIEWLLDGGPSSGAPPPSK

SEQ ID NO: 457 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFIEWLLDGGPSSGAPPPSK

SEQ ID NO: 458 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYSKYLDARRAKEFVQYLLEGGPSSGAPPPSK

SEQ ID NO: 459 - protein, 40 aa, synthetic construct
  SITE 2: note = D-alanine
  MOD_RES 40: note = AminoEthoxyEthoxyAcetic Acyl-MaleimidoPropionic Acyl
HAQGTFTSDYAKYLDARRAKEFVQYLLEGGPSSGAPPPSK

SEQ ID NO: 460 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFVQWLVNGGPSSGAPPPSK

SEQ ID NO: 461 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFIAWLLDGGPSSGAPPPSK

SEQ ID NO: 462 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFIEYLVNGGPSSGAPPPSK

SEQ ID NO: 463 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFIAYLLEGGPSSGAPPPSK

SEQ ID NO: 464 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFIEWLVNGGPSSGAPPPSK

SEQ ID NO: 465 - protein, 40 aa, synthetic construct
  SITE 2: note = D-alanine
  MOD_RES 40: note = AminoEthoxyEthoxyAcetic Acyl-MaleimidoPropionic Acyl
HAEGTFTSDYAKYLDARRAKEFVQWLKAGGPSSGAPPPSK

SEQ ID NO: 466 - protein, 40 aa, synthetic construct
  SITE 2: note = D-alanine
  MOD_RES 40: note = AminoEthoxyEthoxyAcetic Acyl-AminoEthoxyEthoxyAcetic Acyl-MaleimidoPropionic Acyl
YAEGTFTSDYAIYLDAQAQQDFVQWLLAGGPSSGAPPPSK

SEQ ID NO: 467 - protein, 40 aa, synthetic construct
  SITE 2: note = D-alanine
  MOD_RES 40: note = AminoEthoxyEthoxyAcetic Acyl-AminoEthoxyEthoxyAcetic Acyl-MaleimidoPropionic Acyl
YAEGTFTSDYSIYLDKIAQQDFVQWLLAGGPSSGAPPPSK

SEQ ID NO: 468 - protein, 40 aa, synthetic construct
  MOD_RES 2: note = Aib
  MOD_RES 40: note = AminoEthoxyEthoxyAcetic Acyl-MaleimidoPropionic Acyl
HXQGTFTSDYSKYLDERAAQDFVQWLLDGGPSSGAPPPSK

SEQ ID NO: 469 - protein, 40 aa, synthetic construct; identical to SEQ ID NO: 355
  MOD_RES 2: note = Aib
  MOD_RES 40: note = AminoEthoxyEthoxyAcetic Acyl-AminoEthoxyEthoxyAcetic Acyl-MaleimidoPropionic Acyl

SEQ ID NO: 470 - protein, 40 aa, synthetic construct; identical to SEQ ID NO: 467
  SITE 2: note = D-alanine
  MOD_RES 40: note = AminoEthoxyEthoxyAcetic Acyl-MaleimidoPropionic Acyl

SEQ ID NO: 471 - protein, 39 aa, synthetic construct; identical to SEQ ID NO: 125
  MOD_RES 2: note = Aib
  MOD_RES 13: note = Aib
  MOD_RES 20: note = AminoEthoxyEthoxyAcetic Acyl-AminoEthoxyEthoxyAcetic Acyl-MaleimidoPropionic Acyl

(19) United States
(12) Patent Application Publication
(10) Pub. No.: US 2026/0183374 A1
(43) Pub. Date: Jul. 2, 2026

(54) Albumin Bound Macromolecule Tri-Agonist Activating GLP 1/GIP/Glucagon Receptors And Methods Therefor

(71) Applicant: AlbuNext, LLC, El Segundo, CA (US)

(72) Inventors: Patrick Soon-Shiong, Culver City, CA (US); Martin Robitaille, Saint-Colomban (CA); Bing Song, El Segundo, CA (US)

(73) Assignee: AlbuNext, LLC, El Segundo, CA (US)

(21) Appl. No.: 19/447,763

(22) Filed: Jan. 13, 2026

Related U.S. Application Data
(63) Continuation of application No. 18/753,928, filed on Jun. 25, 2024, now Pat. No. 12,551,541.
(60) Provisional application No. 63/523,324, filed on Jun. 26, 2023.
(60) Provisional application No. 63/619,584, filed on Jan. 10, 2024.
(60) Provisional application No. 63/624,692, filed on Jan. 24, 2024.
(60) Provisional application No. 63/625,169, filed on Jan. 25, 2024.
(60) Provisional application No. 63/551,315, filed on Feb. 8, 2024.
(60) Provisional application No. 63/551,334, filed on Feb. 8, 2024.
(60) Provisional application No. 63/551,370, filed on Feb. 8, 2024.
(60) Provisional application No. 63/561,187, filed on Mar. 4, 2024.
(60) Provisional application No. 63/564,426, filed on Mar. 12, 2024.
(60) Provisional application No. 63/564,941, filed on Mar. 13, 2024.
(60) Provisional application No. 63/566,870, filed on Mar. 18, 2024.
(60) Provisional application No. 63/567,402, filed on Mar. 19, 2024.
(60) Provisional application No. 63/568,988, filed on Mar. 22, 2024.
(60) Provisional application No. 63/634,379, filed on Apr. 15, 2024.
(60) Provisional application No. 63/631,777, filed on Apr. 30, 2024.
(60) Provisional application No. 63/641,782, filed on May 2, 2024.
(60) Provisional application No. 63/650,095, filed on May 21, 2024.

(51) Int. Cl. A61K 39/00; A61K 38/00; A61K 38/38; A61P 3/10; C07K 14/47; C07K 14/765; C07K 16/28

(52) U.S. Cl. CPC A61K 39/0005; A61K 38/385; A61P 3/10; C07K 14/4722; C07K 14/765; C07K 16/28; A61K 38/00; A61K 2039/505; C07K 2317/75; C07K 2317/92; C07K 2319/31

(57) ABSTRACT

A pharmaceutical composition comprises a GPCR agonist fusion protein in which a GPCR agonist peptide is covalently coupled to albumin via a linker in a manner that is resistant to a retro-Michael addition. Advantageously, compositions presented herein avoid decoupling of the agonist form the albumin while retaining the agonist in a steric relationship to the albumin that allows for effective binding and activation of the GPCR while also enabling gp60-mediated transcytosis and FcRn-mediated albumin recycling. These properties enable ultra-low dosages for the GPCR agonist fusion protein to give a therapeutic effect while substantially reducing or even entirely avoiding adverse effects otherwise commonly associated with unbound agonists. Moreover, these properties also enable transport of the GPCR agonist fusion protein across the blood brain barrier and therefore allow treatment of neural disorders.

This application is a continuation of allowed US patent application with the Ser. No. 18/753,928, filed Jun. 25, 2024, which claims priority to US Provisional patent applications with the Ser. Nos. 63/523,324, filed Jun. 26, 2023, 63/619,584, filed Jan. 10, 2024, 63/624,692, filed Jan. 24, 2024, 63/625,169, filed Jan. 25, 2024, 63/551,315, filed Feb. 8, 2024, 63/551,334, filed Feb. 8, 2024, 63/551,370, filed Feb. 8, 2024, 63/561,187, filed Mar. 4, 2024, 63/564,426, filed Mar. 12, 2024, 63/564,941, filed Mar. 13, 2024, 63/566,870, filed Mar. 18, 2024, 63/567,402, filed Mar. 19, 2024, 63/568,988, filed Mar. 22, 2024, 63/631,777, filed Apr. 9, 2024, 63/634,379, filed Apr. 15, 2024, 63/641,782, filed May 2, 2024, and 63/650,095, filed May 21, 2024, each of which are incorporated by reference herein.

### SEQUENCE LISTING

The content of the XML file of the sequence listing named 1026900063US-M, which is 1,005,176 bytes in size was created on Aug. 7, 2024, and electronically submitted via Patent Center along with the present application, and is incorporated by reference in its entirety.

### FIELD OF THE INVENTION

The field of the invention is pharmaceutical composition and methods, particularly as they relate to agonists, di-agonists, and tri-agonists of glucagon-like peptide 1 (GLP-1) receptors, glucose-dependent insulinotropic polypeptide (GIP) receptors, and/or glucagon receptors.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Obesity, a global health crisis, is escalating at an alarming rate. As of 2020, 41.9% of adults in the U.S. were classified as obese. Globally, over 1 billion people, including 650 million adults, are affected by obesity. This condition is associated with a multitude of health complications, including hyperglycemia, type 2 diabetes, hypertension, dyslipidemia, obstructive sleep apnea, atherosclerosis, osteoarthritis, urinary incontinence, non-alcoholic steatohepatitis, cardiovascular diseases, certain cancers, and premature mortality. Furthermore, obesity negatively impacts both physical and mental health, leading to a diminished quality of life and reduced cardiorespiratory fitness, which in turn heightens the risk of cardiovascular diseases and overall mortality.

Similarly, diabetes mellitus has become an increasingly common illness with 537 million adults are affected worldwide in 2021 with a prediction of 783 million by 2045. Of these patients, approximately 90% have type 2 (or non-insulin dependent) diabetes. Often related to obesity, this disease is characterized by increased blood glucose levels, decreased insulin action, and impaired glucose tolerance. Treatment usually includes a diet, weight loss, and exercise regimen as well as the administration of anti-diabetic agents including metformin to decrease hepatic glucose output, sulfonylurea and metformin to increase insulin secretion, and/or thiazolidinediones to enhance insulin sensitivity.

While these drug treatment options are often effective, the disease may progress in spite of treatment, and patients may eventually require daily administration of insulin. Hence, new therapeutic strategies in the form of insulinotropic peptides have been developed for the treatment of Type 2 diabetes, including analogs of peptides that control blood glucose levels, such as glucagon-like peptide 1 (GLP-1), gastric inhibitory polypeptide (GIP), Glucagon, and Amylin.

GLP-1 is a naturally occurring hormone that is released by L cells in the lower intestine in response to nutrient ingestion. In addition to having potent insulinotropic effects, GLP-1 has been shown to suppress glucagon, stimulate pancreatic β-cell proliferation, inhibit gastric emptying, and decrease gastrointestinal (GI) motility, all actions that contribute to the glucose-dependent postprandial maintenance of normal blood glucose levels and regulation of appetite. Because of these pleiotropic effects, GLP-1 analogs make attractive candidates for the treatment of Type 2 diabetes.

One major drawback to the use of native GLP-1 is the extremely short half-life of several minutes, due to cleavage by the ubiquitous proteolytic enzyme dipeptidyl peptidase IV at an N-terminal alanine residue. In contrast to the short-lived GLP-1 peptide, exendin-4 (a 39 amino acid peptide agonist of the glucagon-like peptide 1 (GLP-1) receptor) is resistant to dipeptidyl peptidase IV degradation and has a circulating half-life of approximately 33 minutes in humans. Exendin-4 induces similar physiological effects as GLP-1, through the GLP-1 receptor, and is more potent than native GLP-1. Synthetic exendin-4 (Byetta) has been used as an adjunctive therapy with oral antidiabetic agents in the treatment of non-insulin dependent diabetes mellitus (T2DM). In an approach to further extend half-life of exendin-4, the agonist was encapsulated in poly-(D-L-Lactide-Co-Glycolide) microspheres (Bydureon).

The need for even longer active formulations led to the development of recombinant proteins such as the fusion protein described in CA 2434237 (not marketed) and the fusion protein albiglutide (TANZEUM™, Glaxosmithkline, discontinued), as described in U.S. Pat. No. 7,141,547. Albiglutide is an albumin fusion protein of GLP-1 in which a tandem repeat of Gly8 GLP-1 is directly fused to the N-terminus of human serum albumin. Here, the tandem repeat was intended to improve affinity for the GLP-1 receptor by creating a longer distance between albumin and the distal GLP-1 peptide. While the half-life was extended to 6-8 days, making it suitable for once weekly dosing, the potency of albiglutide was significantly reduced (GLP-1 receptor affinity of albiglutide is 20 nM compared with 0.02 nM for exenatide), most likely due to a combination of the Gly8 modification and the direct covalent fusion with the albumin. When administered as a monotherapy, the decrease in HbA1c from baseline at the 30 mg weekly dose was −0.7% at week 52. At the 50 mg weekly dose, the change from baseline of HbA1c was −0.9%. Despite these high doses of GLP-1 at 30 and 50 mg, the change in HbA1c from baseline never achieved a decrease≥1%. Based on these findings, it was taught by Knudsen et al (Knudsen L B, Lau J. The Discovery and Development of Liraglutide and Semaglutide. Front Endocrinol (Lausanne). 2019 Apr. 12; 10:155. doi: 10.3389/fendo.2019.00155. PMID: 31031702; PMCID: PMC6474072.) that a GLP-1 agonist, when fused to albumin, will not produce an effective composition, under the premise that GLP-1 agonists could bind to albumin or to the GLP-1 receptor, but not to both. Specifically, the industry taught against irreversible binding to albumin and instead implemented the idea “was to build on reversible binding to albumin as a solution for the systemic protraction of GLP-1 analogs. The main challenge identified in earlier studies was that strong binding to albumin had a negative impact on the potency of compounds for the GLP-1R, due to competition between binding to albumin and binding to the receptor). The theory was that only the free fraction in the plasma that was not bound to albumin would be available to activate the GLP-1R. Therefore, the stronger the affinity to albumin the smaller the free and active circulating fraction of the GLP-1 peptide. This phenomenon had previously been observed with liraglutide analogs, where there was a clear trend for longer fatty acids, with improved affinity for albumin, to be associated with diminished potency for the GLP-1R”.

On the basis of this assumption, a modified approach was adopted that made use of reversible non-covalent binding of GLP-1 analogs to albumin. In this approach, GLP-1 agonists were acylated to so facilitate non-covalent binding of the acylated GLP-1 agonists to the seven fatty acid binding sites in albumin, resulting in extended half-lives of these compositions. Examples for such approaches include VICTOZA™ (liraglutide, Novo Nordisk, albumin with modified GLP-1 agonist), OZEMPIC™/WEGOVY™ (semaglutide, Novo Nordisk, albumin with modified GLP-1 agonist), MOUNJARO™/ZEPBOUND™ (tirzepatide, Eli Lilly, albumin with modified GLP-1/GIP dual agonist), Pemvidutide (AltImmune, albumin with modified GLP-1/Glucagon dual agonist), efinopegdutide (Merck, albumin with modified GLP-1/Glucagon dual agonist), and subroutine (Zealand, Boehringer, albumin with modified GLP-1/Glucagon dual agonist). While at least some of these formulations provided an increased stability and serum half-life, dissociation of the modified agonist from the albumin carrier is nevertheless required for receptor activation, leading once more to renal clearance of the agonist. Moreover, numerous adverse effects such as nausea, vomiting, and diarrhea are common with formulations having reversible and non-covalent binding of GLP-1 analogs. To increase tolerability of such compositions, it is routinely recommended to titrate a subject up to a tolerated therapeutic dosage, which can take several weeks.

In still further known compositions, exendin-4 is covalently bound via a linker to Cys₃₄ of albumin as is described in WO 2007/053946, WO 2009/075859, and WO 2011/109787, and CA 2501421 and CA 2550050 teach further insulinotropic agents with a linker that can be coupled to albumin. While conceptually attractive, similar difficulties as observed with the acylated GLP-1 agonists described above remain, as the covalent bond at the Cys₃₄ of albumin is in such compounds generally subject to a retro-Michael addition reaction and so liberates the formerly bound exendin-4, leading once more to fast renal clearance and potential adverse effects due to the unbound GLP-1 agonist.

Thus, even though various compositions and methods of GPCR agonists, and especially GLP-1, GIP, and glucagon receptor agonists, are known in the art, all or almost all of them suffer from several drawbacks. In particular, small agonist peptides such as GLP-1 are quickly degraded by endopeptidases and as such require frequent administration. Likewise, small agonist peptide analogs such as exendin-4 are more stable, but still undergo fast renal clearance, once more necessitating frequent administration. On the other hand, GLP-1 albumin direct fusion proteins have significantly longer serum half-life times, but due to steric hindrance with the target receptors, require very high dosages. Alternatively, fatty acid-modified GLP-1 and GLP-1 analogs can be non-covalently bound to hydrophobic pockets in albumin, which requires their dissociation from albumin first to activate their target receptor. Unfortunately, dissociated free fatty acid-modified GLP-1 and analogs thereof lead to adverse effects. Moreover, such albumin-associated fatty acid-modified GLP-1 and GLP-1 analogs will not penetrate the blood brain barrier (BBB). Similarly, where linker-bound exendin-4 is coupled to Cys₃₄ in albumin via Michael addition, free exendin-4 is once more encountered due to a retro-Michael addition reaction, leading once more to fast Exendin-4 clearance and adverse effects due to unbound agonists.

Therefore, there is still a need to provide improved compositions in which a GPCR agonist has upon administration a high stability in plasma/serum, retains agonist activity at low concentrations while being bound to a carrier, and in which the GPCR agonist can upon administration readily move from the blood compartment into a target tissue or even pass the blood brain barrier into neural tissues.

### SUMMARY OF THE INVENTION

The inventive subject matter is directed to various compositions and methods of GPCR agonists that have upon administration a high stability in plasma/serum, that retain agonist activity while being bound to a carrier, and in which the GPCR agonist can upon administration readily move from the blood compartment into a target tissue or even pass the blood brain barrier into neural tissues. Moreover, owing to their particular chemical structure, compounds presented herein can be administered at an ultra-low dose with high therapeutic efficacy without titration and with minima adverse effects such as nausea, vomiting, and/or diarrhea. Indeed, the compositions presented herein have a heretofore unrealized high therapeutic index in their class.

In one aspect of the inventive subject matter, the inventors contemplate a method of treating a subject that includes a step of administering a therapeutically effective amount of a pharmaceutical composition to the subject in need thereof, wherein the pharmaceutical composition comprises a plurality of fusion proteins in each of which a Class B GPCR agonist peptide is, via a linker, covalently bound to a Cys34 amino acid of an albumin. In such methods, the majority of the linkers with the GPCR agonist peptides is bound to the Cys34 amino acid in a retro-Michael resistant manner while the fusion protein has a conformation that allows binding of the GPCR agonist to a GPCR receptor to thereby activate the GPCR receptor.

In some embodiments, the therapeutically effective amount is equal or less than 800 mcg, or equal or less than 250 mcg, or even equal or less than 150 mcg of the GPCR agonist peptide, which is typically administered by subcutaneous injection or intranasal administration once weekly. In further embodiments, at last 70%, or at least 90% of the linkers with the GPCR agonist peptides are bound to the Cys34 amino acid in the retro-Michael resistant manner, and/or the albumin is recombinant human albumin. Moreover, it should be appreciated that administration of the therapeutically effective amount may be performed without titration. In yet further embodiments, the albumin may be a conformationally modified albumin, and/or the pharmaceutical composition may further comprise additional albumin having an unreacted Cys34 group.

In particularly contemplated methods, the fusion protein is retro-Michael resistant albenatide (AB-013-AEEA-succinimide (SEQ ID NO:2)-albumin), retro-Michael resistant albenatidex (AB-014-AEEA-succinimide (SEQ ID NO:185)-albumin), retro-Michael resistant albugiptide (AB-029-AEEA-succinimide (SEQ ID NO:7)-albumin), or retro-Michael resistant albutide (AB-044-AEEA-succinimide (SEQ ID NO:223)-albumin). Viewed from a different perspective, the GPCR agonist peptide may be a GLP-1 agonist peptide, a GLP-1/GIP dual agonist peptide, or a GLP-1/GIP/GcG tri-agonist peptide.

For example, the subject in such method may be diagnosed with a metabolic disorder such as NIDDM (non-insulin dependent diabetes mellitus), NASH (non-alcoholic steatohepatosis/steatohepatitis), chronic kidney disease, obesity cachexia, sarcopenia, diabetic kidney disease, diabetic foot ulcer, and diabetic retinopathy. Or the subject may be diagnosed with a neurodegenerative disease such as Parkinson's Disease, Alzheimer's Disease, peripheral neuropathy, multiple sclerosis, and amyotrophic lateral sclerosis (ALS). In still further contemplated examples, the subject may be diagnosed with an autoimmune or inflammatory disease such as Chron's disease, irritable bowel syndrome (IBS), ulcerative colitis, and polycystic ovary syndrome.

In another aspect of the inventive subject matter, the inventors also contemplate a method of treating a disease in a subject that includes a step of administering a therapeutically effective amount of a pharmaceutical composition to the subject in need thereof, wherein the pharmaceutical composition comprises fusion proteins in which a Class B GPCR agonist peptide is, via a linker, covalently coupled to a Cys34 amino acid in an albumin. In such methods, it is contemplated that the fusion proteins have a conformation that allows gp60-mediated transcytosis and neonatal Fc receptor (FcRn) recycling, thereby enabling transport of substantially all of the fusion protein across a blood brain barrier while the GPCR agonist peptide remains bound to the linker and the albumin.

For example, particularly contemplated neurodegenerative diseases include Parkinson's Disease, Alzheimer's Disease, peripheral neuropathy, multiple sclerosis, and amyotrophic lateral sclerosis (ALS). Moreover, it is generally preferred that the therapeutically effective amount in such methods will be equal or less than 1,000 mcg, or equal or less than 250 mcg of the GPCR agonist peptide administered once weekly. As will be readily appreciated, the pharmaceutical composition may be administered by subcutaneous injection or intranasal administration.

In further contemplated embodiments, the albumin is recombinant human albumin, which may be conformationally modified albumin. Alternatively, or additionally, the pharmaceutical composition may further comprise albumin having an unreacted Cys34 group. Particularly preferred fusion proteins include retro-Michael resistant albenatide (AB-013-AEEA-succinimide (SEQ ID NO:2)-albumin), retro-Michael resistant albenatidex (AB-014-AEEA-succinimide (SEQ ID NO:185)-albumin), retro-Michael resistant albugiptide (AB-029-AEEA-succinimide (SEQ ID NO:7)-albumin), and retro-Michael resistant albutide (AB-044-AEEA-succinimide (SEQ ID NO:223)-albumin).

In a still further aspect of the inventive subject matter, the inventors contemplate a method of treating a metabolic or neurodegenerative disorder in a subject that includes a step of administering a therapeutically effective amount of a pharmaceutical composition to the subject in need thereof, wherein the pharmaceutical composition comprises a fusion protein in which a Class GPCR agonist peptide is, via a linker, covalently coupled to a Cys34 amino acid in a conformationally modified albumin, and wherein the therapeutically effective amount of the Class B GPCR agonist peptide is equal or less than 800 mcg.

In some embodiments of such methods, the subject is diagnosed with a metabolic disorder (e.g., NIDDM (non-insulin dependent diabetes mellitus), NASH (non-alcoholic steatohepatosis/steatohepatitis), chronic kidney disease, obesity cachexia, sarcopenia, diabetic kidney disease, diabetic foot ulcer, or diabetic retinopathy), a neurodegenerative disease (e.g., Parkinson's Disease, Alzheimer's Disease, peripheral neuropathy, multiple sclerosis, or amyotrophic lateral sclerosis (ALS)), or an autoimmune or inflammatory disease (e.g., Chron's disease, irritable bowel syndrome (IBS), ulcerative colitis, or polycystic ovary syndrome).

Moreover, it is contemplated that the GPCR agonist peptide may be a GLP-1 agonist peptide, a GLP-1/GIP dual agonist peptide, or a GLP-1/GIP/GcG tri-agonist peptide. However, especially preferred fusion proteins include retro-Michael resistant albenatide (AB-013-AEEA-succinimide (SEQ ID NO:2)-albumin), retro-Michael resistant albenatidex (AB-014-AEEA-succinimide (SEQ ID NO:185)-albumin), retro-Michael resistant albugiptide (AB-029-AEEA-succinimide (SEQ ID NO:7)-albumin), and retro-Michael resistant albutide (AB-044-AEEA-succinimide (SEQ ID NO:223)-albumin).

In yet another aspect of the inventive subject matter, the inventors also contemplate a method of reducing an adverse effect of administration of a Class B GPCR agonist peptide in a subject. Such methods will typically include a step of administering a pharmaceutical composition to the subject in need thereof in which the GPCR agonist peptide is covalently bound to a Cys34 amino acid of an albumin in a stereopreferred or stereoselective configuration, and wherein the pharmaceutical composition is substantially free of unbound GLP-1 agonist peptide.

Among other options, it is contemplated that the GPCR agonist peptide is a GLP-1 agonist peptide, a GLP-1/GIP dual agonist peptide, or a GLP-1/GIP/GcG tri-agonist peptide. Most typically, the adverse effect is at least one of nausea, vomiting, and diarrhea, and the composition is administered to provide equal or less than 800 mcg, or equal or less than 250 mcg of the GPCR agonist peptide once weekly. As noted above, especially preferred fusion proteins include retro-Michael resistant albenatide (AB-013-AEEA-succinimide (SEQ ID NO: 2)-albumin), retro-Michael resistant albenatidex (AB-014-AEEA-succinimide (SEQ ID NO: 185)-albumin), retro-Michael resistant albugiptide (AB-029-AEEA-succinimide (SEQ ID NO: 7)-albumin), and retro-Michael resistant albutide (AB-044-AEEA-succinimide (SEQ ID NO: 223)-albumin).

In some contemplated embodiments, the albumin is conformationally modified, wherein the pharmaceutical composition has a pH of pH<7.0, while in other embodiments the pharmaceutical composition further comprises albumin having an unreacted Cys34 group, and wherein the pharmaceutical composition has a pH of pH<7.0.

In view of the above the inventors therefore also contemplate a method of treating a subject diagnosed with a disease that includes a step of administering a therapeutically effective amount of a pharmaceutical composition to the subject in need thereof, wherein the pharmaceutical composition comprises (a) a retro-Michael resistant albenatide (AB-013-AEEA-succinimide (SEQ ID NO:2)-albumin); (b) a retro-Michael resistant albenatidex (AB-014-AEEA-succinimide (SEQ ID NO:185)-albumin); (c) a retro-Michael resistant albugiptide (AB-029-AEEA-succinimide (SEQ ID NO:7)-albumin); or (d) a retro-Michael resistant albutide (AB-044-AEEA-succinimide (SEQ ID NO:223)-albumin). Most typically, the disease is NIDDM (non-insulin dependent diabetes mellitus), NASH (non-alcoholic steatohepatosis/steatohepatitis), or obesity.

In preferred embodiments, the therapeutically effective amount is equal or less than 400 mcg, or equal or less than 200 mcg of the GPCR agonist peptide administered once weekly. Most typically the pharmaceutical composition will be administered by subcutaneous injection or intranasal administration. Moreover, it is contemplated that the pharmaceutical composition has a pH of pH<7.0 (e.g., pH of between 4.0 and 6.0), and/or that the albumin in the albenatide, albenatidex, albugiptide, or albutide is a conformationally modified albumin.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 depicts exemplary results for EC₅₀ values in CHO cells and blood glucose changes in mice, demonstrating significant half-life of selected tested compounds and biological activity by the tri-agonist receptor of the albumin bound peptide.

FIG. 2 depicts exemplary results for EC₅₀ values in CHO cells, demonstrating biological signaling activity by the tri-agonist receptor of selected albumin bound peptides with different anchoring sites.

FIG. 3 depicts exemplary results for blood glucose changes in diabetic mice, demonstrating significant half-life and biological activity of selected tested compounds.

FIG. 4 depicts exemplary results for blood glucose changes in diabetic mice, demonstrating significant half-life and biological activity of further selected tested compounds with different anchoring sites.

FIG. 5 depicts exemplary results for blood glucose changes in diabetic mice, demonstrating significant half-life and biological activity of still further selected tested compounds with different anchoring sites and demonstrating that different amino acid modifications in the tri-agonist macromolecule peptide result in different potencies and biological activity of blood glucose change.

FIG. 6 depicts exemplary results for blood glucose changes in diabetic mice, demonstrating significant half-life and biological activity of additional selected tested compounds with different anchoring sites.

FIG. 7 depicts exemplary results for blood glucose changes in diabetic mice, demonstrating that despite being reactive to three receptors, the half-life of the GLP-1/GIP/glucagon tri-agonist peptide has equivalent half-life to a single agonist HSA bound molecules.

FIG. 8 depicts exemplary results for EC₅₀ data in CHO cells and blood glucose changes in diabetic mice, demonstrating that specific amino acid sequences are necessary for binding affinity and biological activity of albumin bound macromolecule peptide with tri-agonist receptor activity. No biological of CJC-1922-HSa with EC₅₀ of 489.5 nM.

FIG. 9 depicts exemplary results for in vivo physiological activity (food and water consumption) and blood glucose changes in diabetic mice for various covalently albumin bound peptides with tri-agonist activity.

FIG. 10 depicts further exemplary results for in vivo physiological activity (food and water consumption) and blood glucose changes in diabetic mice for various covalently albumin bound peptides with tri-agonist activity

FIG. 11 depicts exemplary results for blood glucose changes in diabetic mice, showing prolonged half-life of albumin bound macromolecules with capabilities to activate multiple receptors (GLP-1/GIP/Glucagon).

FIG. 12 depicts an exemplary pharmacokinetics profile of ultra-low dose AB-013 in Type 2 diabetic human subjects with prolonged and flattened profile in the plasma, showing that the pharmacokinetic profile is dose-proportional.

FIG. 13 depicts exemplary results for is an ultra-low dose of fully-bound exendin-4 (AB-013: Albenatide) with low and flattened pk profile.

FIG. 14 depicts exemplary results for further pharmacokinetic data for AB-013 that are consistent with previous pharmacokinetic data, once more showing a low and flattened plasma profile at low concentrations over several weeks.

FIG. 15 depicts exemplary results for Type 2 diabetic patients at various dosage regimens establishing that a 130 mcg (0.13 mg) dose has therapeutic effect on lowering fasting plasma glucose vs. placebo.

FIG. 16 depicts exemplary results for Type 2 diabetic patients at various dosage regimens establishing that a 130 mcg (0.13 mg) dose has therapeutic effect on lowering HbA1c vs. Placebo.

FIG. 17 depicts exemplary results for Type 2 diabetic patients at various dosage regimens establishing that a 130 mcg (0.13 mg) dose has a high therapeutic index with minimized GI adverse events.

FIG. 18 depicts exemplary results establishing that a 0.13 mg ultra-low dose of AB-013 could achieve a therapeutic effect in T2D subjects on metformin. Here, an ultra-low dose of 0.13 mg of albenatide resulted in a HbA1c reduction by week 5 with reduction of >1% by Week 9 and sustained this reduction to the end of the study at Week 17, which confirms the hypothesis that an albumin-bound Exendin-4 binds to the GLP-1 receptor with therapeutic effect at an ultralow dose (0.13 mg compared to 2.4 mg of semaglutide)).

FIG. 19 depicts exemplary results for GI Adverse Effect (Nausea, Vomiting, Diarrhea) at 0.13 mg and 0.20 mg Versus Placebo in T2D Subjects. Here, GI adverse events were less than <5% by week 9 (vomiting, nausea, and diarrhea) in subjects receiving 0.13 mg without titration. At this ultra-low dose, HbA1c decreased>1%. Thus, highest therapeutic index achieved with demonstration that even at this ultra-low dose, a therapeutic activity is observed.

FIG. 20 depicts exemplary results for subjects with T2D on Metformin to demonstrate the efficacy of the lowest dose of AB-013 to reduce HbA1c over 52 weeks.

FIG. 21 depicts exemplary results for subjects with T2D (naïve) to demonstrate the efficacy of the lowest dose of AB-013 to reduce HbA1c over 52 weeks.

FIG. 22 depicts exemplary results for GI Adverse Events (Vomiting, Nausea, Diarrhea) over 24 weeks at the ultra-low dose of 0.13 mg without titration, establishing that the GI adverse events are minimal with less than 2% by week 4 for vomiting, nausea, and diarrhea.

FIG. 23 depicts exemplary results for additional compounds tested in mice with blood glucose change over time (0-6 hrs and 0-72 hrs).

FIG. 24 depicts exemplary results for additional compounds tested in mice with blood glucose change over time (0-6 hrs and 0-48 hrs).

FIG. 25 depicts exemplary results for additional compounds tested in mice with blood glucose change over time (0-6 hrs and 0-48 hrs).

FIG. 26 depicts exemplary results for additional compounds tested in mice with blood glucose change over time (0-6 hrs and 0-72 hrs).

FIG. 27 depicts exemplary results for additional compounds tested in mice with blood glucose change over time (0-6 hrs and 0-72 hrs).

FIG. 28 depicts exemplary results for additional compounds tested in mice with blood glucose change over time (0-48).

FIG. 29 depicts exemplary results for additional compounds tested in mice with blood glucose change over time (0-24 hrs and 0-48 hrs).

FIG. 30 depicts exemplary results for additional compounds tested in mice with blood glucose change over time (0-48 hrs).

FIG. 31 depicts exemplary results for additional compounds tested in mice with blood glucose change over time (0-48 hrs).

FIG. 32 is an exemplary dosage comparison of known GLP-1 agonist compounds and AB-013.

FIG. 33 is an exemplary comparison of AB-013 with semaglutide, illustrating higher therapeutic index and other benefits.

FIGS. 34A and 34B are graphs depicting exemplary results for average body weight changes of mice after dosing with test compounds and control.

FIG. 35 is a graph depicting exemplary results for cumulative food consumption of mice after dosing with test compounds and controls.

FIG. 36 is a graph depicting exemplary results for cumulative water consumption of mice after dosing with test compounds and controls.

FIG. 37 is a graph depicting exemplary results for average blood glucose of mice after dosing with test compounds and controls.

FIG. 38 is a graph depicting exemplary results for liver weight of mice after dosing with test compounds and controls.

FIG. 39 is a graph depicting exemplary results for AST values of mice at the end of the study after dosing with test compounds and controls.

FIG. 40 is a graph depicting exemplary results for ALT values of mice at the end of the study after dosing with test compounds and controls.

FIG. 41 is a is a graph depicting exemplary results for total cholesterol of mice at the end of the study after dosing with test compounds and controls.

FIG. 42 is a is a graph depicting exemplary results for LDL cholesterol of mice at the end of the study after dosing with test compounds and controls.

FIG. 43 is a graph depicting exemplary results for serum IL-la of mice after dosing with test compounds and controls.

FIG. 44 is a graph depicting exemplary results for serum IL-6 of mice after dosing with test compounds and controls.

FIG. 45 are exemplary photomicrographs of liver sections from mice treated with test compounds and controls.

### DETAILED DESCRIPTION

The inventors have discovered therapeutic compositions and methods in which various GPCR agonist peptides, and especially incretins/insulinotropic agents such as GLP-1, GIP, Glucagon (GcG), Amylin, NPY2, Neuropeptide Y, and PYY, and derivatives, analogs, and fragments thereof, can be delivered as a fusion protein to a subject in need thereof at a heretofore unrealized high therapeutic index in their class. Indeed, owing to their particular chemical structure, contemplated compounds and compositions allow for ultra-low dosages at once weekly administration with minimal adverse effects such as nausea, vomiting, and/or diarrhea, and without the need for titration to a tolerated dose. In this context, it should be appreciated that the term ‘fusion protein’ in conjunction with the compounds presented herein includes proteins in which a first peptide portion (e.g., agonist peptide) is covalently bound to a second peptide portion (e.g., albumin) via a linker moiety, where the linker may be a third peptide portion or a non-peptide portion (e.g., AEEA-MPA). Thus, the term ‘fusion protein’ in conjunction with the compounds presented herein may also be referred to as ‘bioconjugate’ or ‘conjugate’

In this context it should be appreciated that as the GPCR agonist peptides remain at all times covalently bound to albumin, numerous shortfalls of currently known agonist peptide formulations such as very high dosages and adverse effects can be avoided. In addition, it should be recognized that such irreversible coupling also allows the fusion protein to readily move from the blood compartment to a target tissue and even pass the blood brain barrier (via gp60 transcytosis and FcRn recycling) into neural tissues, thereby increasing half-live time and reducing required dosage and adverse effects.

In contrast, currently known GLP-1 agonists are formulated as unbound agonists, as PEG-conjugates, as fusion proteins in which the agonist and a protein carrier form a contiguous polypeptide backbone, or as fatty acid derivatives that are non-covalently bound to albumin via hydrophobic interaction with the hydrophobic binding pockets in albumin. Representative compounds for such agonist compositions include Semaglutide (NovoNordisk, GLP-1 agonist), Tirzepatide (Eli Lilly, Dual Agonist) Retatrutide (Eli Lilly, Tri-Agonist), Danuglipron (Pfizer, Abandoned), Albiglutide (GSK, Abandoned), Lixisenatide (Sanofi/Zealand, Abandoned), Efinopegdutide (Merck, GLP-1, Glucagon agonist), Cafraglutide (Amgen, GLP-1 agonist), Survodutide (Boehringer Ingelheim, Glucagon/GLP-1 dual agonist), Pemvidutide (Altimmune), Pegapamodutide (OPKO Health, Mid-stage trials).

Unfortunately, these formulations share numerous common drawbacks, such as the need to titrate a subject to a maximum tolerated or therapeutically effective dose, and a variety of gastrointestinal side effects such as nausea, vomiting, and diarrhea. For example, currently approved products (e.g., semaglutide and tirzepatide) are associated with high incidence of nausea and vomiting, often resulting in patient discontinuance of drug. Likewise, pre-clinical and clinical experience with free exendin-4 (e.g., exenatide) exhibited early onset and high incidence of vomiting as was the case with free unbound GLP-1. More particularly, free unbound exendin-4 (exenatide), even at low doses of 5 mcg and 10 mcg was reported to result in 44% incidence of nausea, and at 100 mcg resulted in violent adverse events including projectile vomiting. In other studies, free unbound exendin-4 at 0.1 μmol/kg infused resulted in nausea and vomiting, and at 0.16 μmol/kg per minute, free unbound GLP-1 resulted in vomiting within eight minutes, consistent with high peak levels at time of administration.

In contrast, the inventors have now discovered that fusion proteins can be prepared by use of a linker that irreversibly and covalently connects albumin to a Class B GPCR agonist peptide. For example, in some preferred embodiments, one end of the linker will be covalently bound to a reactive group at the C-terminus of the agonist peptide (e.g., via an amide bond) while the other end of the linker is covalently bound to the albumin at Cys₃₄ via a retro-Michael resistant thioether bond in which the carbon atom of the coupling group in the linker has a stereospecific configuration that renders the bond retro-Michael resistant within the context of the hydrophobic pocket in which the Cys₃₄ is located. In other preferred embodiments, one end of the linker will be covalently bound to a reactive group in a side chain of an amino acid at an intermediate position in the agonist peptide (e.g., via an amide bond) and the other end of the linker will be covalently bound to the albumin at Cys₃₄ via a retro-Michael resistant thioether bond in which the carbon atom of the coupling group in the linker has a stereospecific configuration that renders the bond retro-Michael resistant within the context of the hydrophobic pocket in which the Cys₃₄ is located. In this context it should be appreciated that suitable linkers will provide a rotational degree of freedom and a steric distance between the agonist peptide and the albumin so as to enable binding of the agonist peptide to the target receptor and activation of the target receptor while remining covalently bound to the albumin.

Advantageously, contemplated retro-Michael resistant albumin-bound insulinotropic agonists will transcytose rapidly to the tissue microenvironment of the pancreas and the brain, leaving the plasma compartment through the gp60/caveolin-1/caveoli pathway with long-acting agonist via FcRn rescue. As such, contemplated compounds and compositions can take advantage of cell mediated transport, thereby allowing for ultra-low dose and ultra-low plasma concentration to so enable a therapeutic effect with a high therapeutic index and a substantially lower GI adverse event profile.

As will be appreciated, especially contemplated peptide agonists suitable for use in conjunction with the teachings presented herein will particularly include GPCR agonists (and especially Class B GPCR agonists) and all derivatives, analogs, and fragments thereof. Therefore, and among other contemplated peptide agonists, GLP-1, GIP, Glucagon (GcG), Amylin, NPY2, Neuropeptide Y, and PYY, and derivatives, analogs, and fragments are especially preferred. It should furthermore be appreciated that the peptide agonists contemplated herein may have binding and activation specificity towards a single receptor (mono-agonist), two receptors (dual-agonist), or three receptors (tri-agonist). In this context, it should be particularly appreciated that contrary to conventional wisdom, compounds can be produced in which the ligand is covalently and irreversibly bound to the albumin, and in which the same ligand can bind to and activate two, or even three distinct G-protein coupled receptors (GLP-1, GIP, and/or GcG receptors). Consequently, it should be recognized that the biological activity of fusion proteins containing such peptide agonists may be tailored towards specific uses (e.g., insulinotropic, appetite suppressant, etc.).

In one exemplary fusion protein, retro-Michael resistant albenatide (acting as a GLP-1 agonist) comprises exendin-4 (SEQ ID NO:3) to which on the C-terminus a modified lysine is covalently coupled, wherein the modified lysine contains a linker with a coupling group. In this example, the modified lysine forms a peptide bond with the C-terminal serine of exendin-4, and the modified lysine further includes an AEEA-MPA group that is covalently bound to the epsilon amino group of the lysine via an amide bond. Upon reaction of the MPA coupling group with the thiol group of Cys₃₄ in the albumin in a stereopreferred or stereoselective manner as described in more detail below, the retro-Michael resistant albenatide (AB-013-AEEA-succinimide-albumin). Of course, it should be appreciated that various alternative linker moieties may be used, and suitable alternative linkers with coupling groups include MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, and (AEEEA) 2-OA-(bromo) MPA (with MPA denoting maleimidopropionic acid, and with OA denoting 8-aminooctanoate). Where desired, the modified lysine may also be coupled to the C-terminal serine via a single AEEA group. In such case, the modified lysine may once more include coupling groups such as MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, or (AEEEA) 2-OA-(bromo) MPA.

In another exemplary fusion protein, and following the same approach, a retro-Michael resistant albenatidex (AB-014-AEEA-succinimide (SEQ ID NO:185)-albumin) (acting as a GLP-1 agonist) comprises a chimeric GLP-1/exendin-4 portion to which on the C-terminus a modified lysine is covalently coupled, wherein the modified lysine contains a linker with a coupling group. In this example, the modified lysine forms a peptide bond with the C-terminal serine of chimeric peptide, and the modified lysine further includes an AEEA-MPA group that is covalently bound to the epsilon amino group of the lysine via an amide bond as is shown in SEQ ID NO.185. Upon reaction of the MPA coupling group with the thiol group of Cys86 in the albumin in a stereopreferred or stereoselective manner as described in more detail below, the retro-Michael resistant albenatidex is formed (AB-014-AEEA-succinimide-albumin). As noted above, it should be appreciated that various alternative linker moieties may be used, and suitable alternative linkers with coupling groups include MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA)₂-OA-MPA, and (AEEEA)₂-OA-(bromo) MPA (with MPA denoting maleimidopropionic acid, and with OA denoting 8-aminooctanoate). Where desired, the modified lysine may also be coupled to the C-terminal serine via a single AEEA group. In such case, the modified lysine may once more include coupling groups such as MPA, (bromo) MPA, AEEA, AEEA-MPA, AEEA-(bromo) MPA, (AEEA)₂-MPA, (AEEA)₂-(bromo) MPA, AEEEA-OA-MPA, AEEEA-OA-(bromo) MPA, AEEA-OA-AEEA-MPA, AEEA-OA-AEEA-(bromo) MPA, (AEEEA)₂-OA-MPA, or (AEEEA)₂-OA-(bromo) MPA.

In still another exemplary fusion protein, and once more following the same approach, a retro-Michael resistant albugiptide (AB-029-AEEA-succinimide (SEQ ID NO:7)-albumin) (acting as a GIP/GLP-1 dual agonist) comprises a modified exendin-4 portion to which on the C-terminus a modified lysine is covalently coupled, wherein the modified lysine contains a linker with a coupling group. In this example, the modified lysine forms a peptide bond with the C-terminal serine of the modified exendin-4, and the modified lysine further includes an AEEA-MPA group that is covalently bound to the epsilon amino group of the lysine via an amide bond as is shown in SEQ ID NO:7. Once more, upon reaction of the MPA coupling group with the thiol group of Cys₃₄ in the albumin in a stereopreferred or stereoselective manner as described in more detail below, the retro-Michael resistant albugiptide is formed (AB-029-AEEA-succinimide-albumin). As already noted above, it should be appreciated that various alternative linker moieties may be used, and suitable alternative linkers with coupling groups include MPA, (bromo) MPA, AEEA, AEEA-MPA, AEEA-(bromo) MPA, (AEEA)₂-MPA, (AEEA)₂-(bromo) MPA, AEEEA-OA-MPA, AEEEA-OA-(bromo) MPA, AEEA-OA-AEEA-MPA, AEEA-OA-AEEA-(bromo) MPA, (AEEEA)₂-OA-MPA, and (AEEEA)₂-OA-(bromo) MPA (with MPA denoting maleimidopropionic acid, and with OA denoting 8-aminooctanoate). Where desired, the modified lysine may also be coupled to the C-terminal serine via a single AEEA group. In such case, the modified lysine may once more include coupling groups such as MPA, (bromo) MPA, AEEA, AEEA-MPA, AEEA-(bromo) MPA, (AEEA)₂-MPA, (AEEA)₂-(bromo) MPA, AEEEA-OA-MPA, AEEEA-OA-(bromo) MPA, AEEA-OA-AEEA-MPA, AEEA-OA-AEEA-(bromo) MPA, (AEEEA)₂-OA-MPA, or (AEEEA)₂-OA-(bromo) MPA.

In a still further exemplary fusion protein, and yet again following the same approach, a retro-Michael resistant albutide (AB-044-AEEA-succinimide (SEQ ID NO:223)-albumin) (acting as a GIP/GLP-1/GcG tri-agonist) comprises a modified exendin-4 portion to which on the C-terminus a modified lysine is covalently coupled, wherein the modified lysine contains a linker with a coupling group. In this example, the modified lysine forms a peptide bond with the C-terminal serine of the modified exendin-4, and the modified lysine further includes an AEEA-MPA group that is covalently bound to the epsilon amino group of the lysine via an amide bond as is shown in SEQ ID NO:223. Once more, upon reaction of the MPA coupling group with the thiol group of Cys₃₄ in the albumin in a stereopreferred or stereoselective manner as described in more detail below, the retro-Michael resistant albutide albutide (AB-044-AEEA-succinimide (SEQ ID NO:223)-albumin) is formed. Additionally, a closely related retro-Michael resistant fusion protein, AB-045-AEEA-succinimide (SEQ ID NO:69)-albumin, is also particularly contemplated. Once more, it should be appreciated that various alternative linker moieties may be used, and suitable alternative linkers with coupling groups include MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, and (AEEEA) 2-OA-(bromo) MPA (with MPA denoting maleimidopropionic acid, and with OA denoting 8-aminooctanoate). Where desired, the modified lysine may also be coupled to the C-terminal serine via a single AEEA group. In such case, the modified lysine may once more include coupling groups such as MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, and (AEEEA) 2-OA-(bromo) MPA.

As will be readily appreciated, contemplated fusion proteins need not be limited to those shown above, and a number of other GPCR agonist peptides are also suitable for use herein, so long as the alternative GPCR agonist peptide is covalently bound via a linker to the albumin in a retro-Michael resistant manner. For example, especially suitable agonist peptides are provided in Table 1 below in which the amino acids and modifying characters are as provided in the sequence listing of this application. Moreover, it should be appreciated that (1) sequences in the table without linker modifications may also include a linker modification as described herein, particularly where the linker modification is covalently attached to an epsilon amino group of a lysine, and suitable linker modifications include MPA, (bromo) MPA, AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, and (AEEEA) 2-OA-(bromo) MPA; (2) any specific linker modification shown in the table below (e.g., AEEA-MPA) as indicated by a particular modifying character may be replaced with any other linker modification contemplated herein (e.g., AEEA-AEEA-MPA); (3) the C-terminal amino acid may be covalently attached to a solid phase (e.g., modified Ramage resin), preferably via a spacer group; (4) any sequence in the table below may be covalently coupled via a linker as described herein to albumin at Cys₃₄, preferably in a retro-Michael resistant manner (e.g., in a stereopreferred or stereoselective manner); and (5) any sequence in the table below having a linker modification is also contemplated as an agonist peptide sequence without the linker modification.

TABLE 1

| SEQ | |
|---|---|
| ID | |
| NO: | SEQUENCE |
| 1 | HaEGTFTSDVSSYLEGQAAKEFIAWLVKGRK* |
| 2 | HGEGTFTSDLSKQMEEEAVRLFIEWLKNGGPSSGAPPPSK* (Albenatide - |
| | AB013 when bound to Cys₃₄ in retro-Michael resistant/ |
| | stereopreferred or stereoselective manner via |
| | maleimide reactive group) |
| 3 | HGEGTFTSDLSKQMEEEAVRLFIEWLKNGGPSSGAPPPS |
| 4 | HAEGTFTSDVSSYLEGQAAKEFIAWLVKGR |
| 5 | YAEGTFISDYSIAMDKIHQQDFVNWLLAQKGKKNDWKHNITQ |
| 6 | HSQGTFTSDYSKYLDSRRAQDFVQWLMNT |
| 7 | HaEGTFTSDVSSYLEGQAAKEFIAWLVNGGPSSGAPPPSK* (Albugiptide - |
| | AB-029 when bound to Cys₃₄ in retro-Michael resistant/ |
| | stereopreferred or stereoselective manner via maleimide |
| | reactive group) |
| 8 | HaEGTFTSDYAKYLDARRAKEFIAWLVKGRPSSGAPPPSK* |
| 9 | HGEGTFTSDLSKQMEEEAVRLFIEWLKNGGPK*SGAPPPS |
| 10 | HaEGTFTSDVSK*YLEGQAAKEFIAWLVKGRPSSGAPPPS |
| 11 | HaEGTFTSDVSSYLEK*QAAKEFIAWLVKGRPSSGAPPPS |
| 12 | HGEGTFTSDLSKQMEEEAVRLFIEWLKNGGPK*SGAPPPS |
| 13 | HaEGTFTSDINKVLDTIAAKEFIAWLVKGRPSSGAPPPSK* |
| 14 | YAibEGTFTSDYSIYLDKQAAAibEFVQWLLAGGPSSGAPPPSK″ |
| 15 | YGGGTFTSDSFFYLELSHAKDFINWLQLGaPSSGAPPPSK* |
| 16 | HAibAibGTFTSDEMNYLDDWMQAibAFVNWLVAibGIPSSGAPPPSK* |
| 17 | FAibPGTFTSDGHNYLDWQDAKEFIQWLGWGVPSSGAPPPSK* |
| 18 | YAibMGTFTSDPQIAibLEMKEQAibDFINWLNDGFPSSGAPPPSK* |
| 19 | HaPGTFTSDNHEAibLDTYRQAibDFINWLDTGVPSSGAPPPSK* |
| 20 | YaNGTFTSDIWAYLDSSFAQDFVAWLYIGKPSSGAPPPSK* |
| 21 | HAibDGTFTSDFEHYLEDAVQKAFIAWLSTGVPSSGAPPPSK* |
| 22 | FGDGTFTSDLRGAibLEIMPQAibAFVNWLTSGKPSSGAPPPSK* |
| 23 | HaaGTFTSDsMIYLDNAHQQEFIQWLFNGPPSSGAPPPSK* |
| 24 | HaIGTFTSDsTAibYLEaYKQAibDFVQWLHSGGPSSGAPPPSK* |
| 25 | YsWGTFTSDaGYYLDMsFQQAFIQWLKAGaPSSGAPPPSK* |
| 26 | YGRGTFTSDWIYAibLDTaPQKEFVEWLHsGAPSSGAPPPSK* |
| 27 | YaMGTFTSDSEEAibLEMVYQQAFVAWLPVGGPSSGAPPPSK* |
| 28 | FssGTFTSDWDWAibLDIAibIAAibEFINWLVYGYPSSGAPPPSK* |
| 29 | FAibGGTFTSDDMRYLEPKGQQAFIQWLWVGQPSSGAPPPSK* |
| 30 | YAibSGTFTSDVETYLDLLIAKEFIAWLGAGAibPSSGAPPPSK* |
| 31 | HGRGTFTSDHQFAibLDSMIQAibEFIQWLHYGIPSSGAPPPSK* |
| 32 | YaRGTFTSDFINAibLESGVAKAFVNWLQEGaPSSGAPPPSK* |
| 33 | HsFGTFTSDGWMYLDMNEQKAFVAWLYaGaPSSGAPPPSK* |
| 34 | FsRGTFTSDHYRAibLDQSKAAibEFIEWLESGNPSSGAPPPSK* |
| 35 | FsGGTFTSDHHRYLDQIPQKDFIQWLEFGKPSSGAPPPSK* |
| 36 | FaQGTFTSDaNMAibLDMFaQKAFINWLFQGGPSSGAPPPSK* |
| 37 | FaAGTFTSDVTaAibLEVAibQQKAFIAWLNLGQPSSGAPPPSK* |
| 38 | HaIGTFTSDHMAibYLDsDPAQDFIEWLMPGSPSSGAPPPSK* |
| 39 | HAibNGTFTSDIIHYLDPIVQKAFIEWLNGGAPSSGAPPPSK* |
| 40 | HGEGTFTSDMAibQAibLDREIAKEFVEWLFFGYPSSGAPPPSK* |
| 41 | HsPGTFTSDPDsAibLDENHQQAFVAWLNsGMPSSGAPPPSK* |
| 42 | FsaGTFTSDsLDYLEssEQAibEFINWLAAGVPSSGAPPPSK* |
| 43 | YaAibGTFTSDKDPYLEAibTYAAibEFVAWLVsGRPSSGAPPPSK* |
| 44 | FGRGTFTSDAVMAibLDAMQAKAFIEWLWIGIPSSGAPPPSK* |
| 45 | HaaGTFTSDVDYAibLEWKIQQDFIEWLSTGLPSSGAPPPSK* |
| 46 | YsFGTFTSDIAGYLDLEAibAAibAFVQWLAHGKPSSGAPPPSK* |
| 47 | HsHGTFTSDEVQYLDMDGQQEFINWLDLGYPSSGAPPPSK* |
| 48 | HsRGTFTSDLQGAibLEYADQAibDFVNWLAibTGFPSSGAPPPSK* |
| 49 | HGFGTFTSDGIDAibLEaITAKEFIAWLaIGKPSSGAPPPSK* |
| 50 | HAibTGTFTSDIalAibLDYKGAAibAFVQWLDAGEPSSGAPPPSK* |
| 51 | HsLGTFTSDLAibMAibLDVNIAAibDFIAWLMIGYPSSGAPPPSK* |
| 52 | YGDGTFTSDKHRAibLEISQQKEFVQWLLsGLPSSGAPPPSK* |
| 53 | FaaGTFTSDQTTAibLDAGSAKDFVNWLVAibGAibPSSGAPPPSK* |
| 54 | YAibGGTFTSDEPPYLDAWTQAibEFVEWLVRGWPSSGAPPPSK* |
| 55 | HaGGTFTSDGSRAibLDIaTQQDFIEWLLEGHPSSGAPPPSK* |
| 56 | HsSGTFTSDPLKYLDHGNQQAFVNWLISGSPSSGAPPPSK* |
| 57 | HAibTGTFTSDISFYLEEYVAQEFVAWLQAGsPSSGAPPPSK* |
| 58 | HsaGTFTSDaYEAibLEPAibDQKAFIAWLWHGLPSSGAPPPSK* |
| 59 | YsRGTFTSDATLAibLEGSKQKAFINWLLEGIPSSGAPPPSK* |
| 60 | FGWGTFTSDLEKAibLELARAQAFVEWLKVGSPSSGAPPPSK* |
| 61 | FAibMGTFTSDPYPYLEWEaAQAFINWLsGGSPSSGAPPPSK* |
| 62 | HaAibGTFTSDMPTAibLDHIQQQDFVAWLVQGIPSSGAPPPSK* |
| 63 | FasGTFTSDHSDAibLELKAQQEFVNWLRNGRPSSGAPPPSK* |
| 64 | HGQGTFTSDYSKYLDARRAQDFVEWLKNGGPSSGAPPPSK* |
| 65 | HAibQGTFTSDYSKAibLDKRRAKDFVEWLKNGGPSSGAPPPSK* |
| 66 | HAibQGTFTSDYSKYLDKRRAKDFVEWLKNGGPSSGAPPPSK* |
| 67 | YAibQGTFTSDYSImelLDKIAQAibAFIEYLLEGGPSSGAPPPSK* |
| 68 | YAibEGTFTSDYSIAibLDKIAQKAFVQWLIAGGPSSGAPPPSK* |
| 69 | HaEGTFTSDYAKYLDARRAKEFIAWLVNGGPSSGAPPPSK* |
| 70 | YAibEGTFTSDYSIYLDKQAAAibEFVQWLLAGGPSSGAPPPSK* |
| 71 | HAibHGTFTSDLSKLLEEQRQAibEFIEWLKAaGPPPSAibKPPPK* |
| 72 | HaMGTFTSDRHWYLDMSHQK*AFVQWLAYGNPSSGAPPPS |
| 73 | YasGTFTSDWGRYLELLIQK*EFVNWLIIGAPSSGAPPPS |
| 74 | HGKGTFTSDsAIAibLEVsaAK*AFIEWLaHGDPSSGAPPPS |
| 75 | HasGTFTSDILEAibLDQAAAK*EFVEWLaHGFPSSGAPPPS |
| 76 | HaYGTFTSDYMAibAibLDFLQQK*DFVAWLFMGVPSSGAPPPS |
| 77 | HAibsGTFTSDAibAYYLDNTTAK*DFIQWLDAGPPSSGAPPPS |
| 78 | FAibKGTFTSDDaVYLEAibHRAK*EFVEWLFGGsPSSGAPPPS |
| 79 | YAibHGTFTSDLTVAibLDEIKAK*EFVEWLAibVGPPSSGAPPPS |
| 80 | FsWGTFTSDFPaAibLEAibMKAK*DFVEWLLDGNPSSGAPPPS |
| 81 | HAibKGTFTSDDADAibLEWYRQK*AFVQWLPsGIPSSGAPPPS |
| 82 | YAibWGTFTSDSNSAibLEAKMQK*EFVNWLsQGFPSSGAPPPS |
| 83 | FaTGTFTSDDADYLELFsQK*AFIAWLDNGVPSSGAPPPS |
| 84 | YSYGTFTSDWDAAibLESAibMQK*EFVQWLFYGQPSSGAPPPS |
| 85 | YaNGTFTSDTNsAibLDSTQAK*EFVAWLVQGDPSSGAPPPS |
| 86 | FAibIGTFTSDKQAYLEHPRQK*AFVAWLDVGYPSSGAPPPS |
| 87 | HAibRGTFTSDKFIAibLEYHNAK*EFVAWLYKGsPSSGAPPPS |
| 88 | FGHGTFTSDSIWYLENYSQK*EFIEWLEKGPPSSGAPPPS |
| 89 | HsaGTFTSDIRAibYLEIMLQK*EFVEWLaEGVPSSGAPPPS |
| 90 | HAibFGTFTSDMLNYLEENsQK*EFVNWLQLGMPSSGAPPPS |
| 91 | FGHGTFTSDIWIYLEVQTAK*DFINWLSWGEPSSGAPPPS |
| 92 | HsGGTFTSDSGPYLDKTDQK*AFINWLPIGNPSSGAPPPS |
| 93 | YsIGTFTSDHsPAibLDHLFAK*DFVEWLENGDPSSGAPPPS |
| 94 | FGYGTFTSDKEGAibLEQsAibAK*EFIQWLPHGPPSSGAPPPS |
| 95 | FsWGTFTSDFAibSAibLDTTRAK*DFVEWLIRGNPSSGAPPPS |
| 96 | HAibPGTFTSDHEKYLEMVsAK*DFIAWLRAibGDPSSGAPPPS |
| 97 | YaAGTFTSDTIaAibLDNaAAK*DFIAWLIQGYPSSGAPPPS |
| 98 | YAibLGTFTSDTIHAibLEFEAQK*DFINWLKAGEPSSGAPPPS |
| 99 | YAibQGTFTSDAMIYLDTPDAK*EFIAWLAibIGGPSSGAPPPS |
| 100 | YGAGTFTSDHAibGAibLDIAibQQK*EFVQWLETGTPSSGAPPPS |
| 101 | YaVGTFTSDLSEYLEMNIAK*DFVQWLLVGGPSSGAPPPS |
| 102 | YGPGTFTSDRYsYLEQHMAK*EFIQWLGPGHPSSGAPPPS |
| 103 | YAibRGTFTSDMRVYLEETLQK*AFVQWLIaGDPSSGAPPPS |
| 104 | YsAibGTFTSDFMEAibLDKKIQK*DFIQWLWYGSPSSGAPPPS |
| 105 | FAibsGTFTSDELRYLEEMHQK*AFIQWLAibTGFPSSGAPPPS |
| 106 | YGPGTFTSDWAKYLEGRDAK*AFINWLsQGSPSSGAPPPS |
| 107 | HGVGTFTSDHHFAibLEIIMAK*AFIEWLWHGaPSSGAPPPS |
| 108 | HGIGTFTSDVPAYLDPAaAK*DFIQWLVSGGPSSGAPPPS |
| 109 | HsNGTFTSDRRKAibLEPPIAK*EFVQWLIAibGIPSSGAPPPS |
| 110 | FAibTGTFTSDTMaYLDYTHAK*DFIAWLIDGKPSSGAPPPS |
| 111 | YaTGTFTSDsVQYLEHPMQK*AFVAWLHTGRPSSGAPPPS |
| 112 | YAibTGTFTSDEEWYLDNWMQK*AFVQWLSNGAibPSSGAPPPS |
| 113 | YsIGTFTSDGNKAibLEAibGRAK*AFIAWLQYGRPSSGAPPPS |
| 114 | YsPGTFTSDMYVYLEPGDAK*EFVQWLKNGGPSSGAPPPS |
| 115 | FsIGTFTSDLMaAibLERAAQK*EFVNWLIIGsPSSGAPPPS |
| 116 | YAibNGTFTSDIEQYLEPMVQK*EFVQWLSPGNPSSGAPPPS |
| 117 | YGWGTFTSDSFsAibLDWMMAK*AFIQWLIVGIPSSGAPPPS |
| 118 | HsPGTFTSDAibTHYLDNDQQK*DFVNWLPEGWPSSGAPPPS |
| 119 | YGaGTFTSDNHTAibLEsFAAK*EFIQWLNSGQPSSGAPPPS |
| 120 | YaMGTFTSDRLRAibLDQFSAK*AFVNWLSWGEPSSGAPPPS |
| 121 | FAibaGTFTSDSLAibAibLDHHNQK*AFVEWLaPGLPSSGAPPPS |
| 122 | HGQGTFTSDYSKYLDK*RRAQDFVEWLKNGGPSSGAPPPS |
| 123 | HAibQGTFTSDYSKAibLDKRRAK*DFVEWLKNGGPSSGAPPPS |
| 124 | HAibQGTFTSDYSKYLDK*RRAQDFVEWLKNGGPSSGAPPPS |
| 125 | YAibEGTFTSDYSIAibLDKIAQK*AFVQWLIAGGPSSGAPPPS |
| 126 | YAibQGTFTSDYSImeILDKIAQK*AFIEYLLEGGPSSGAPPPS |
| 127 | HaEGTFTSDYAKYLDARRAK*EFIAWLVNGGPSSGAPPPS |
| 128 | YaWGTFTSDsKDYLEFK*WAAibEFIQWLDPGDPSSGAPPPS |
| 129 | FAibYGTFTSDINVAibLDIK*WAAibAFINWLPGGIPSSGAPPPS |
| 130 | FaaGTFTSDRQEAibLDVK*LAAibEFVQWLVSGDPSSGAPPPS |
| 131 | FaRGTFTSDSMQAibLETK*RQQEFVNWLsMGVPSSGAPPPS |
| 132 | YaHGTFTSDTNRAibLESK*AAAibEFIAWLEVGAPSSGAPPPS |
| 133 | FAibGGTFTSDFRsAibLDsK*IAAibEFINWLFKGFPSSGAPPPS |
| 134 | YAibNGTFTSDMMPYLEPK*HQKDFVEWLTSGDPSSGAPPPS |
| 135 | HaPGTFTSDIIHYLETK*IAAibEFIQWLKRGSPSSGAPPPS |
| 136 | FsVGTFTSDAibSAAibLDLK*IAAibEFINWLFaGFPSSGAPPPS |
| 137 | YAibaGTFTSDGNMYLDKK*HAKDFIEWLsSGPPSSGAPPPS |
| 138 | YaIGTFTSDPsHAibLEVK*NQKEFVQWLNRGIPSSGAPPPS |
| 139 | FsTGTFTSDFVEAibLEIK*EQKAFIEWLAQGGPSSGAPPPS |
| 140 | FGTGTFTSDFDGYLEDK*aAKAFIQWLsGGMPSSGAPPPS |
| 141 | HsWGTFTSDWYIAibLEEK*NAAibDFIAWLYAGYPSSGAPPPS |
| 142 | FGPGTFTSDKKFAibLELK*GAKAFIAWLAibAibGFPSSGAPPPS |
| 143 | HsNGTFTSDaNsYLEPK*AAAibDFVNWLDAibGWPSSGAPPPS |
| 144 | FsTGTFTSDPWVYLDSK*QQQAFVEWLHFGNPSSGAPPPS |
| 145 | FaEGTFTSDWYPAibLDLK*SAAibDFVQWLYGGPPSSGAPPPS |
| 146 | FGIGTFTSDTARAibLDDK*DQKEFVQWLNDGMPSSGAPPPS |
| 147 | FGYGTFTSDKSTYLDEK*IQAibDFVEWLNDGsPSSGAPPPS |
| 148 | HGSGTFTSDAAibPYLDQK*GAQDFIAWLDGGPPSSGAPPPS |
| 149 | HaIGTFTSDPPVAibLEQK*HQQEFVAWLDPGLPSSGAPPPS |
| 150 | YaYGTFTSDVNKYLDPK*TAAibAFVEWLaQGIPSSGAPPPS |
| 151 | FaIGTFTSDRREYLEPK*EQAibDFIEWLRDGGPSSGAPPPS |
| 152 | YGNGTFTSDAWIYLDDK*LQKEFVQWLRLGNPSSGAPPPS |
| 153 | YaPGTFTSDaaAAibLEDK*YQAibAFIAWLAibQGWPSSGAPPPS |
| 154 | YGaGTFTSDIRAYLDEK*WAKAFVAWLVsGMPSSGAPPPS |
| 155 | YGWGTFTSDPINAibLDLK*AibQKDFVNWLPMGAPSSGAPPPS |
| 156 | HsNGTFTSDsYaYLERK*DQKAFIEWLWSGPPSSGAPPPS |
| 157 | YsMGTFTSDQGKYLEAK*SAQAFINWLMAibGAPSSGAPPPS |
| 158 | YaFGTFTSDLTRAibLDAibK*TAKAFIEWLDaGMPSSGAPPPS |
| 159 | YAibKGTFTSDAibIYYLDWK*NAAibAFVNWLHIGIPSSGAPPPS |
| 160 | HAibQGTFTSDWsAibAibLEKK*VQAibEFVNWLAibTGDPSSGAPPPS |
| 161 | FssGTFTSDDRaAibLDSK*aAQAFVAWLAibAGTPSSGAPPPS |
| 162 | YaIGTFTSDMKIYLDEK*aAKAFVQWLANGsPSSGAPPPS |
| 163 | FGQGTFTSDYDQAibLDNK*PQAibDFIEWLMYGEPSSGAPPPS |
| 164 | FsMGTFTSDLDRAibLDWK*IQKDFVQWLVSGTPSSGAPPPS |
| 165 | YsVGTFTSDLHsYLDLK*GAQDFVNWLVWGQPSSGAPPPS |
| 166 | HAibIGTFTSDDVYYLDLK*PQKEFVEWLGLGSPSSGAPPPS |
| 167 | FGVGTFTSDVMIAibLDIK*EQKEFINWLQSGEPSSGAPPPS |
| 168 | HAibFGTFTSDHAibsYLEsK*MAQDFINWLIIGsPSSGAPPPS |
| 169 | HaGGTFTSDPAKYLDSK*VQQEFVAWLAFGAibPSSGAPPPS |
| 170 | YaNGTFTSDVsFAibLEYK*DQKDFIQWLAsGQPSSGAPPPS |
| 171 | FsVGTFTSDNRQYLDVK*NQAibDFIQWLPWGIPSSGAPPPS |
| 172 | HAibVGTFTSDKETYLDIK*KAAibDFVEWLSTGRPSSGAPPPS |
| 173 | FGWGTFTSDDFNAibLEYK*HAAibEFVNWLDVGQPSSGAPPPS |
| 174 | FaYGTFTSDQSYAibLDYK*TAKDFIQWLTKGRPSSGAPPPS |
| 175 | FGPGTFTSDDAibIYLEaK*NAQAFIEWLFSGQPSSGAPPPS |
| 176 | YAibIGTFTSDSsIAibLDSK*AQAibDFIQWLLFGQPSSGAPPPS |
| 177 | HsGGTFTSDPYGAibLEQK*MAKEFVEWLMTGKPSSGAPPPS |
| 178 | HaEGTFTSDYAKYLDAK*RAKEFIAWLVNGGPSSGAPPPS |
| 179 | YAibQGTFTSDYSImelLDKK*AQAibAFIEYLLEGGPSSGAPPPS |
| 180 | HGQGTFTSDYSKYLDK*RRAQDFVEWLKNGGPSSGAPPPS |
| 181 | HAibQGTFTSDYSKAibLDK*RRAQDFVEWLKNGGPSSGAPPPS |
| 182 | HAibQGTFTSDYSKYLDK*RRAQDFVEWLKNGGPSSGAPPPS |
| 183 | X1X2 X3GTFTSDX4X5 X6X7 LX8X9 X10X11X12X13 X14 |
| | FX15X16WLX17X18GX19PSSGAPPPSX20 |
| 184 | X1X2 X3GTFTSDX4X5 X6X7 LX8X9 X10X11X12X13 X14 |
| | FX15X16WLX17X18GX19PSSGAPPPSX20X21 |
| 185 | HAibEGTFTSDVSSYLEGQAAKEFIAWLVKGRK* (Albenatidex - |
| | AB-014 when bound to Cys₃₄ in retro-Michael |
| | resistant/stereopreferred or stereoselective |
| | manner via maleimidereactive group) |
| 186 | HAibEGTFTSDVSSYLEGQAAKEFIAWLVKGRK\hat{ } |
| 187 | HAibEGTFTSDVSSYLEGQAAKEFIAWLVKGRK″ |
| 188 | HAibEGTFTSDVSSYLEGQAAKEFIAWLVKGRK′ |
| 189 | HAibEGTFTSDVSSYLEGQAAKEFIAWLVKGR9K* |
| 190 | HGEGTFTSDLSKQMEEEAVRLFIEWLKNGGPSSGAPPPSK\hat{ } |
| 191 | HGEGTFTSDLSKQMEEEAVRLFIEWLKNGGPSSGAPPPSK″ |
| 192 | HGEGTFTSDLSKQMEEEAVRLFIEWLKNGGPSSGAPPPSK′ |
| 193 | HGEGTFTSDLSKQMEEEAVRLFIEWLKNGGPSSGAPPPS9K* |
| 194 | HsQGTFTSDYSKYLDSRRAQDFVQWLMNTGPSSGAPPPSK* |
| 195 | HsQGTFTSDYSKYLDSRRAQDFVQWLMNTGPSSGAPPPSK\hat{ } |
| 196 | HsQGTFTSDYSKYLDSRRAQDFVQWLMNTGPSSGAPPPSK″ |
| 197 | HsQGTFTSDYSKYLDSRRAQDFVQWLMNTGPSSGAPPPSK′ |
| 198 | HsQGTFTSDYSKYLDSRRAQDFVQWLMNTGPSSGAPPPS9K* |
| 199 | YaEGTFISDYSIAMDKIHQQDFVNWLLAQGPSSGAPPPSK* |
| 200 | YaEGTFISDYSIAMDKIHQQDFVNWLLAQGPSSGAPPPSK\hat{ } |
| 201 | YaEGTFISDYSIAMDKIHQQDFVNWLLAQGPSSGAPPPSK″ |
| 202 | YaEGTFISDYSIAMDKIHQQDFVNWLLAQGPSSGAPPPSK′ |
| 203 | YaEGTFISDYSIAMDKIHQQDFVNWLLAQGPSSGAPPPS9K* |
| 204 | HAEGTFTSDVSSYLEGQAAK*EFIAWLVRGR |
| 205 | HAibEGTFTSDVSSYLEGQAAK*EFIAWLVRGRG |
| 206 | HVEGTFTSDVSSYLEEQAAREFIK*WLVRGRG |
| 207 | HaEGTFTSDVSSYLEGQAAKEFIAWLVNGGPSSGAPPPSK\hat{ } |
| 208 | HaEGTFTSDVSSYLEGQAAKEFIAWLVNGGPSSGAPPPSK″ |
| 209 | HaEGTFTSDVSSYLEGQAAKEFIAWLVNGGPSSGAPPPSK′ |
| 210 | HaEGTFTSDVSSYLEGQAAKEFIAWLVNGGPSSGAPPPS9K* |
| 211 | HaEGTFTSDVASYLEGQAAKEFIAWLVNGGPSSGAPPPSK* |
| 212 | YAibEGTFTSDYSIYLDKQAAAibEFVNWLLAGGPSSGAPPPSK* |
| 213 | HAibQGTFTSDYSKYLDEKKAK*EFVEWLLEGGPSSG |
| 214 | HAibQGTFTSDYSKYLDEK*AAKEFIQWLLQT |
| 215 | HAibQGTFTSDYSKYLDEKRAKEFVQWLMNTC* |
| 216 | HsQGTFTSDK*SKYLDARAAQDFVQWLLDT |
| 217 | HAc4cQGTFTSDYSKYLDERAAKDFIK*WLESA |
| 218 | HsQGTFTSDLSKQK*ESKAAQDFIEWLKAGGPSSGAPPPS |
| 219 | HSQGTFTSDK*SEYLDSERARDFVAWLEAGG |
| 220 | HAibQGTFTSDK*SKYLDERAAQDFVQWLLDGGPSSGAPPPS |
| 221 | HSQGTFTSDK*SKYLDERRAQDFVQWLLDGGPSSGAPPPS |
| 222 | HAibHGTGTSDLSKLK*EEQRQAibEFIEWLKAaGPPSAibKPPPK |
| 223 | HaQGTFTSDYAKYLDARRAKEFIAWLVNGGPSSGAPPPSK* (Albutide - |
| | AB-044 when bound to Cys₃₄ in retro-Michael |
| | resistant/stereopreferred or stereoselective |
| | manner via maleimide reactive group) |
| 224 | HaQGTFTSDYAKYLDARRAKEFIAWLVNGGPSSGAPPPSK\hat{ } |
| 225 | HaQGTFTSDYAKYLDARRAKEFIAWLVNGGPSSGAPPPSK″ |
| 226 | HaQGTFTSDYAKYLDARRAKEFIAWLVNGGPSSGAPPPSK′ |
| 227 | HaEGTFTSDYAKYLDARRAKEFIAWLVNGGPSSGAPPPSK\hat{ } |
| 228 | HaEGTFTSDYAKYLDARRAKEFIAWLVNGGPSSGAPPPSK″ |
| 229 | HaEGTFTSDYAKYLDARRAKEFIAWLVNGGPSSGAPPPSK' |
| 230 | HaEGTFTSDYAKYLDARRAKEFIAWLVNGGPSSGAPPPS9K* |
| 231 | YAEGTFISDYSIAMDKIHQQDFVNWLLAQKGKKNDWKHNITQ |
| 232 | HaEGTFTSDVASYLEGQAAKEFIAWLVNGGPSSGAPPPS9K* |
| 233 | HAibEGTFTSDVSSYLEGQAAK\hat{ }EFIAWLVRGRG |
| 234 | HAibEGTFTSDVSSYLEGQAAKEFIAWLVRGRGK* |
| 235 | HAibEGTFTSDVSSYLEGQAAAibEFIAWLVRGRGK″ |
| 236 | YAibEGTFTSDYSIAibLDKIAQK″AFVQWLIAGGPSSGAPPPS |
| 237 | YAibEGTFTSDYSIAibLDKIAQK\hat{ }AFVQWLIAGGPSSGAPPPS |
| 238 | YAibEGTFTSDYSIYLDKQAAAibEFVQWLLAGGPSSGAPPPSK\hat{ } |
| 239 | YAibEGTFTSDYSIYLDKQAAAibEFVQWLLAGGPSSGAPPPSK# |
| 240 | HAibQGTFTSDYSKYLDEKKAK\hat{ }EFVEWLLEGGPSSG |
| 241 | HAibQGTFTSDYSKYLDEKKAK″EFVEWLLEGGPSSG |
| 242 | HAibQGTFTSDYSKYLDEKKAK#EFVEWLLEGGPSSG |
| 243 | HAibQGTFTSDYSKYLDEKKAKEFVEWLLEGGPSSGK* |
| 244 | HAibQGTFTSDYSKYLDEK\hat{ }AAKEFIQWLLQT |
| 245 | HAibQGTFTSDYSKYLDEK″AAKEFIQWLLQT |
| 246 | HsQGTFTSDLSKQK\hat{ }ESKAAQDFIEWLKAGGPSSGAPPPS |
| 247 | HsQGTFTSDLSKQK#ESKAAQDFIEWLKAGGPSSGAPPPS |
| 248 | HsQGTFTSDLSKQLESKAAQDFIEWLKAGGPSSGAPPPSK* |
| 249 | HsQGTFTSDLSKQLESKAAQDFIEWLKAGGPSSGAPPPSK″ |
| 250 | HSQGTFTSDLSEYLDSERARDFVAWLEAGGK** |
| 251 | YAibQGTFTSDYSIMeLLDKK\hat{ }AQAibAFIEYLLEGGPSSGAPPPS |
| 252 | YAibQGTFTSDYSIMeLLDKK″AQAibAFIEYLLEGGPSSGAPPPS |
| 253 | YAibQGTFTSDYSIMeLLDKK#AQAibAFIEYLLEGGPSSGAPPPS |
| 254 | YAibQGTFTSDYSIMeLLDKIAQAibAFIEYLLEGGPSSGAPPPSK\hat{ } |
| 255 | YAibQGTFTSDYSIMeLLDKIAQAibAFIEYLLEGGPSSGAPPPSK″ |
| 256 | YAibQGTFTSDYSIMeLLDKIAQAibAFIEYLLEGGPSSGAPPPSK# |
| 257 | HSQGTFTSDK\hat{ }SKYLDERRAQDFVQWLLDGGPSSGAPPPS |
| 258 | HSQGTFTSDK″SKYLDERRAQDFVQWLLDGGPSSGAPPPS |
| 259 | HSQGTFTSDYSKYLDERRAQDFVQWLLDGGPSSGAPPPSK* |
| 260 | HSQGTFTSDYSKYLDERRAQDFVQWLLDGGPSSGAPPPSK** |
| 261 | HAibHGTGTSDLSKLK\hat{ }EEQRQAibEFIEWLKAaGPPSAibKPPPK |
| 262 | HAibHGTGTSDLSKLLEEQRQAibEFIEWLKAaGPPSAibKPPPK* |
| 263 | HAibHGTGTSDLSKLLEEQRQAibEFIEWLKAaGPPSAibKPPP9K* |
| 264 | YaEGTFTSDYAKYLDARRAAibEFIAWLVNGGPSSGAPPPSK* |
| 265 | YAibEGTFTSDYAKYLDARRAAibEFIAWLVNGGPSSGAPPPSK* |
| 266 | YAibEGTFTSDYSIAibLDKRRAAibEFIAWLVNGGPSSGAPPPSK* |
| 267 | HaEGTFTSDYSIAibLDKRRAAibEFIAWLVNGGPSSGAPPPSK* |
| 268 | YaEGTFTSDYSIAibLDKRRAAibEFIAWLVNGGPSSGAPPPSK* |
| 269 | YAibEGTFTSDYSIAibLDKIAQAibAFIAWLVNGGPSSGAPPPSK* |
| 270 | HaEGTFTSDYSIAibLDKIAQAibAFIAWLVNGGPSSGAPPPSK* |
| 271 | YaEGTFTSDYSIAibLDKIAQAibAFIAWLVNGGPSSGAPPPSK* |
| 272 | HaEGTFTSDYSIAibLDKIAQAibAFVQWLIAGGPSSGAPPPSK* |
| 273 | YaEGTFTSDYSIAibLDKIAQAibAFVQWLIAGGPSSGAPPPSK* |
| 274 | YAibEGTFTSDYSIAibLDKIAQAibAFVQWLIAGGPSSGAPPPS9K* |
| 275 | HaEGTFTSDYAKYLDARRAAibEFVQWLIAGGPSSGAPPPSK* |
| 276 | YaEGTFTSDYAKYLDARRAAibEFVQWLIAGGPSSGAPPPSK* |
| 277 | YAibEGTFTSDYAKYLDARRAAibEFVQWLIAGGPSSGAPPPSK* |
| 278 | HaEGTFTSDYAKYLDKIAQAibAFVQWLIAGGPSSGAPPPSK* |
| 279 | YaEGTFTSDYAKYLDKIAQAibAFVQWLIAGGPSSGAPPPSK* |
| 280 | YAibEGTFTSDYAKYLDKIAQAibAFVQWLIAGGPSSGAPPPSK* |
| 281 | YAibQGTFTSDYAKYLDARRAKEFIAWLVNGGPSSGAPPPSK* |
| 282 | YaQGTFTSDYAKYLDARRAKEFIAWLVNGGPSSGAPPPSK* |
| 283 | YAibQGTFTSDYSIMeLLDKRRAKEFIAWLVNGGPSSGAPPPSK* |
| 284 | YaQGTFTSDYSIMeLLDKRRAKEFIAWLVNGGPSSGAPPPSK* |
| 285 | HaQGTFTSDYSIMeLLDKRRAKEFIAWLVNGGPSSGAPPPSK* |
| 286 | YAibQGTFTSDYSIMeLLDKIAQAibAFIAWLVNGGPSSGAPPPSK* |
| 287 | YaQGTFTSDYSIMeLLDKIAQAibAFIAWLVNGGPSSGAPPPSK* |
| 288 | HaQGTFTSDYSIMeLLDKIAQAibAFIAWLVNGGPSSGAPPPSK* |
| 289 | YaQGTFTSDYSIMeLLDKIAQAibAFIEYLLEGGPSSGAPPPSK* |
| 290 | HaQGTFTSDYSIMeLLDKIAQAibAFIEYLLEGGPSSGAPPPSK* |
| 291 | YAibQGTFTSDYSIMeLLDKIAQAibAFIEYLLEGGPSSGAPPPS9K* |
| 292 | HaQGTFTSDYAKYLDARRAKEFIEYLLEGGPSSGAPPPSK* |
| 293 | HAibQGTFTSDYAKYLDARRAKEFIEYLLEGGPSSGAPPPSK* |
| 294 | YAibQGTFTSDYAKYLDARRAKEFIEYLLEGGPSSGAPPPSK* |
| 295 | YaQGTFTSDYAKYLDARRAKEFIEYLLEGGPSSGAPPPSK* |
| 296 | HaQGTFTSDYAKYLDKIAQAibAFIEYLLEGGPSSGAPPPSK* |
| 297 | HAibQGTFTSDYAKYLDKIAQAibAFIEYLLEGGPSSGAPPPSK* |
| 298 | YAibQGTFTSDYAKYLDKIAQAibAFIEYLLEGGPSSGAPPPSK* |
| 299 | YaQGTFTSDYAKYLDKIAQAibAFIEYLLEGGPSSGAPPPSK* |
| 300 | HAibQGTFTSDYSIMeLLDKIAQAibAFIEYLLEGGPSSGAPPPSK* |
| 301 | HaQGTFTSDYAKYLDARRAKEFIAWLVNGGPSSGAPPPSK# |
| 302 | HaEGTFTSDYAKYLDAK*RAKEFIAWLVNGGPSSGAPPPS |
| 303 | HaEGTFTSDYAKYLDAK\hat{ }ARAKEFIAWLVNGGPSSGAPPPS |
| 304 | HaEGTFTSDYAKYLDAK&RAKEFIAWLVNGGPSSGAPPPS |
| 305 | HaEGTFTSDYAKYLDAK′RAKEFIAWLVNGGPSSGAPPPS |
| 306 | HaEGTFTSDYAKYLDARRAK\hat{ }EFIAWLVNGGPSSGAPPPS |
| 307 | HaEGTFTSDYAKYLDARRAK&EFIAWLVNGGPSSGAPPPS |
| 308 | HaEGTFTSDYAKYLDARRAK′EFIAWLVNGGPSSGAPPPS |
| 309 | HaQGTFTSDYAKYLDARRAKEFIAWLVNGGPSSGAPPPSK2 |
| 310 | HaQGTFTSDYAKYLDARRAKEFIAWLVNGGPSSGAPPPSK3 |
| 311 | HaQGTFTSDYAKYLDARRAKEFIAWLVNGGPSSGAPPPSK1 |
| 312 | HaQGTFTSDYAKYLDARRAKEFIAWLVNGGPSSGAPPPSC2 |
| 313 | HaQGTFTSDYAKYLDAK2RAKEFIAWLVNGGPSSGAPPPS |
| 314 | HaQGTFTSDYAKYLDAK5RAKEFIAWLVNGGPSSGAPPPS |
| 315 | HaQGTFTSDYAKYLDAK1RAKEFIAWLVNGGPSSGAPPPS |
| 316 | HaQGTFTSDYAKYLDAK4RAKEFIAWLVNGGPSSGAPPPS |
| 317 | YAibQGTFTSDYSILLDKk*AQAibAFIEYLLEGGPSSGAPPPS |
| 318 | HADGSFSDEMNTILDNLAARDFINWLIQTKITD |
| 319 | HSDAVFTDNYTRLRKQMAVKKYLNSILN |
| 320 | KCNTATCATQRLANFLVHSSNNFGAILSSTNVGSNTY |
| 321 | YADAIFTNSYRKVLGQLSARKLLQDIMSR |
| 322 | X1X2X3GTFTSDX4X5X6X7LX8X9X10X11X12X13X14FX15X16 |
| | WLX17X18GX19PSSGAPPPSX20 |
| 323 | X1X2X3GTFTSDX4X5X6X7LX8X9X10X11X12X13X14FX15X16 |
| | WLX17X18GX19PSSGAPPPS |
| 324 | X19PSSGAPPPSX20 |
| 325 | X11X12X13X14FX15X16WLX17X18G |
| 326 | X1X2X3GTFTSDX4X5X6X7LX8X9X10 |
| 327 | X19PSSGAPPPSK |
| 328 | X11X12X13X14FX15X16WLX17X18G-X19PSSGAPPPSX20 |
| 329 | X1X2X3GTFTSDX4X5X6X7LX8X9X10-X11X12X13X14FX15X16 |
| | WLX17X18G-X19PSSGAPPPSX20 |
| 330 | HaEGTFTSDVSSYLEGQAAKEFIAWLVKGRPSSGAPPPSK* |
| 331 | HAEGTFTSDVSSYLEGQAAKEFIAWLVKGRPSSGAPPPSK* |
| 332 | HaEGTFTSDVSSYLEGQAAKEFIAWLVNGGK* |
| 333 | HaEGTFTSDINKVLDTIAAKEFIAWLVKGRK* |
| 334 | HaEGTFTSDAEKAKEAEKAKEFIAWLVKGRK* |
| 335 | HaEGTFTSDAibEKAibKEAibEKAKEFIAWLVKGRK* |
| 336 | HaEGTFTSDAibESAibKEAibEKAibKEFIAWLVKGRPSSGAPPPSK* |
| 337 | HaEGTFTSDVAibSYAibEGQAibAKEFIAWLVKGRPSSGAPPPSK* |
| 338 | HaEGTFTSDVSSYLEGK*AAKEFIAWLVKGRPSSGAPPPS |
| 339 | HaEGTFTSDVSSYLEGQAAKEFIAWLVKGRLLLLLLLLLK* |
| 340 | HaEGTFTSDVSSYLEGQAAKEFIAWLVKGRPEAPTDPEAPTD |
| 341 | HGEGTFTSDLSKQMEEEAVRLFIEWLKNGGGGGGGGGGGK* |
| 342 | HaEGTFTSDVSSYLEGQAAKEFIAWLVKGRK% |
| 343 | HaEGTFTSDVSSYLEGK*AAKEFIAWLVKGR |
| 344 | HaEGTFTSDVSSYLEGQAAKEFIAWLVKGRK@ |
| 345 | HSQGTFTSDYSKYLDSRRAQDFVQWLMNTKRNKNNIA |
| 346 | HaEGTFTSDVSSYLEGQAAKEFIAWLVKGPEAPTDPEAPTD |
| 347 | HaEGTFTSDVSSYLEGQAAKEFIAWLVKGRK** |
| 348 | HaEGTFTSDINKVLDIIAAKEFIAWLVKGRPSSGAPPPSK* |
| 349 | HaEGTFTSDINKVLDIIAAKEFIAWLVKGRK* |
| 350 | HGEGTFTSDLSKQMEEEAVRLFIEWLKNGGPSSGAPPPSK** |
| 351 | HAibEGTFTSDYSKYLDKIRAQEFVAWLMNGGPSSGAPPPSK* |
| 352 | HAibQGTFTSDYSKYLDKIAAQDFVAWLLNGGPSSGAPPPSK* |
| 353 | HAibQGTFTSDYAKYLDKIAAQDFVAWLLDGGPSSGAPPPSK* |
| 354 | HAibQGTFTSDYSKYLDKIAAQDFVAWLLDGGPSSGAPPPSK* |
| 355 | HAibQGTFTSDYSKYLDKIAAQDFVAYLLDGGPSSGAPPPSK* |
| 356 | HAibQGTFTSDLSKYLDEIAVQDFIEWLLDGGPSSGAPPPSK* |
| 357 | HSQGTFTSDYSKYLDSRRAQDFVQWLMNTKRNRNNIA |
| 358 | IKPEAPGEDASPEELNRYYASLRHYLNLVTRQRY |
| 359 | HAibEGTFTSDVSSYLEGQALRHYINWLTRQRY |
| 360 | HGEGTFTSDLSKQMEEEAVRLFIEWLKNGGPSSRHYLNLVTRQRY |
| 361 | HdsQGTFTSDLSKYLEEEAVREFIAWLKNGGPSSGAPPPSRHYLNLVTRQRY |
| 362 | HdsQGTYTNDVSKYXDSRRAQDFIEWLKNGGPSSGAPPPS |
| 363 | HdsQGTYTNDVSKYKDSRRAQDFIEWLKNGGPSSGAPPPSC |
| 364 | HdsQGTFTSDLSKQKDSRRAQDFIEWLKNGGPSSGAPPPSC |
| 365 | HdsQGTYTNDVSKYXDSRRAQDFIEWLKNGGPSSGAPPPSC |
| 366 | PKPEAPGKDASPEEWNRYYADLRHYLNWLTRQRY |
| 367 | IKPEAPGEDASPEELNRYYASLRHYLNWVTRQXY |
| 368 | YAibEGTFTSDYSIYLDKQAAAibAFVQWLIAGGPSSGAPPPSK* |
| 369 | YAibEGTFTSDYSIYLDKQAAAibAFVQWLLAGGPSSGAPPPSK* |
| 370 | YAibEGTFTSDYSIYLDKQAAAibEFVQWLIAGGPSSGAPPPSK* |
| 371 | HaEGTFTSDYSIYLDKQAAAibEFVQWLLAGGPSSGAPPPSK* |
| 372 | YaEGTFTSDYSIYLDKQAAAibEFVQWLLAGGPSSGAPPPSK* |
| 373 | HAibEGTFTSDYSIYLDKQAAAibEFVQWLLAGGPSSGAPPPSK* |
| 374 | HaEGTFTSDYSIYLDKQAAAibAFVQWLIAGGPSSGAPPPSK* |
| 375 | HaEGTFTSDYSIYLDKQAAAibAFVQWLLAGGPSSGAPPPSK* |
| 376 | HaEGTFTSDYSIYLDKQAAAibEFVQWLIAGGPSSGAPPPSK* |
| 377 | YaEGTFTSDYSIYLDKQAAAibAFVQWLIAGGPSSGAPPPSK* |
| 378 | YaEGTFTSDYSIYLDKQAAAibAFVQWLLAGGPSSGAPPPSK* |
| 379 | YaEGTFTSDYSIYLDKQAAAibEFVQWLIAGGPSSGAPPPSK* |
| 380 | YAibEGTFTSDYAKYLDARRAKEFVQWLLAGGPSSGAPPPSK* |
| 381 | YAibEGTFTSDYSKYLDARRAKEFVQWLLAGGPSSGAPPPSK* |
| 382 | YAibEGTFTSDYSKYLDKRRAKEFVQWLLAGGPSSGAPPPSK* |
| 383 | HaEGTFTSDYAKYLDARRAKEFVQWLLAGGPSSGAPPPSK* |
| 384 | HaEGTFTSDYSKYLDARRAKEFVQWLLAGGPSSGAPPPSK* |
| 385 | HaEGTFTSDYSKYLDKRRAKEFVQWLLAGGPSSGAPPPSK* |
| 386 | YaEGTFTSDYAKYLDARRAKEFVQWLLAGGPSSGAPPPSK* |
| 387 | YaEGTFTSDYSKYLDARRAKEFVQWLLAGGPSSGAPPPSK* |
| 388 | YaEGTFTSDYSKYLDKRRAKEFVQWLLAGGPSSGAPPPSK* |
| 389 | HAibEGTFTSDYAKYLDARRAKEFVQWLLAGGPSSGAPPPSK* |
| 390 | HAibEGTFTSDYSKYLDARRAKEFVQWLLAGGPSSGAPPPSK* |
| 391 | HAibEGTFTSDYSKYLDKRRAKEFVQWLLAGGPSSGAPPPSK* |
| 392 | YAibEGTFTSDYAKYLDARRAKAFVQWLIAGGPSSGAPPPSK* |
| 393 | YAibEGTFTSDYSKYLDARRAKAFVQWLIAGGPSSGAPPPSK* |
| 394 | YAibEGTFTSDYSKYLDKRRAKAFVQWLIAGGPSSGAPPPSK* |
| 395 | HaEGTFTSDYAKYLDARRAKAFVQWLIAGGPSSGAPPPSK* |
| 396 | HaEGTFTSDYSKYLDARRAKAFVQWLIAGGPSSGAPPPSK* |
| 397 | HaEGTFTSDYSKYLDKRRAKAFVQWLIAGGPSSGAPPPSK* |
| 398 | YaEGTFTSDYAKYLDARRAKAFVQWLIAGGPSSGAPPPSK* |
| 399 | YaEGTFTSDYSKYLDARRAKAFVQWLIAGGPSSGAPPPSK* |
| 400 | YaEGTFTSDYSKYLDKRRAKAFVQWLIAGGPSSGAPPPSK* |
| 401 | HAibEGTFTSDYAKYLDARRAKAFVQWLIAGGPSSGAPPPSK* |
| 402 | HAibEGTFTSDYSKYLDARRAKAFVQWLIAGGPSSGAPPPSK* |
| 403 | HAibEGTFTSDYSKYLDKRRAKAFVQWLIAGGPSSGAPPPSK* |
| 404 | HaQGTFTSDLSKQLESKAAQDFIEWLKAGGPSSGAPPPSK* |
| 405 | HsQGTFTSDYAKYLDARRAKEFIEWLKAGGPSSGAPPPSK* |
| 406 | HsQGTFTSDYSKYLDARRAKEFIEWLKAGGPSSGAPPPSK* |
| 407 | HsQGTFTSDYAKYLDSRRAKEFIEWLKAGGPSSGAPPPSK* |
| 408 | HsQGTFTSDYSKYLDSRRAKEFIEWLKAGGPSSGAPPPSK* |
| 409 | HaQGTFTSDYSKYLDSRRAKEFIEWLKAGGPSSGAPPPSK* |
| 410 | HaQGTFTSDYAKYLDARRAKEFIEWLKAGGPSSGAPPPSK* |
| 411 | HaQGTFTSDLSKQLESKAAQDFIAWLVNGGPSSGAPPPSK* |
| 412 | HaQGTFTSDYAKYLDARRAKDFIEWLKAGGPSSGAPPPSK* |
| 413 | HaQGTFTSDYSKYLDARRAQEFIEWLKAGGPSSGAPPPSK* |
| 414 | HaQGTFTSDYAKYLDSRAAKEFIEWLKAGGPSSGAPPPSK* |
| 415 | HaQGTFTSDYSKYLDSKRAKEFIEWLKAGGPSSGAPPPSK* |
| 416 | HaQGTFTSDYAKYLEARRAKEFIEWLKAGGPSSGAPPPSK* |
| 417 | HaQGTFTSDYAKQLEARRAKEFIEWLKAGGPSSGAPPPSK* |
| 418 | HAibQGTFTSDYSKYLDERAAQAFIEYLLEGGPSSGAPPPSK* |
| 419 | HaQGTFTSDYSKYLDERAAQDFVQWLLDGGPSSGAPPPSK* |
| 420 | HaQGTFTSDYSKYLDERAAQAFIEYLLEGGPSSGAPPPSK* |
| 421 | HAibQGTFTSDYSKYLDARRAKEFVQWLLDGGPSSGAPPPSK* |
| 422 | HAibQGTFTSDYSKYLDARRAKEFIEYLLEGGPSSGAPPPSK* |
| 423 | HAibQGTFTSDYAKYLDARRAKEFVQWLLDGGPSSGAPPPSK* |
| 424 | HaQGTFTSDYSKYLDARRAKEFVQWLLDGGPSSGAPPPSK* |
| 425 | HaQGTFTSDYAKYLDARRAKEFVQWLLDGGPSSGAPPPSK* |
| 426 | HaQGTFTSDYSKYLDARRAKEFIEYLLEGGPSSGAPPPSK* |
| 427 | HAibQGTFTSDYSKYLDERAAQAFIEYLLDGGPSSGAPPPSK* |
| 428 | HaQGTFTSDYSKYLDERAAQDFVQWLLEGGPSSGAPPPSK* |
| 429 | HaQGTFTSDYSKYLDERAAQAFIEYLLDGGPSSGAPPPSK* |
| 430 | HAibQGTFTSDYSKYLDARRAKEFVQWLLEGGPSSGAPPPSK* |
| 431 | HAibQGTFTSDYSKYLDARRAKEFIEYLLDGGPSSGAPPPSK* |
| 432 | HAibQGTFTSDYAKYLDARRAKEFVQWLLEGGPSSGAPPPSK* |
| 433 | HAibQGTFTSDYAKYLDARRAKEFIEYLLDGGPSSGAPPPSK* |
| 434 | HaQGTFTSDYSKYLDARRAKEFVQWLLEGGPSSGAPPPSK* |
| 435 | HaQGTFTSDYAKYLDARRAKEFVQWLLEGGPSSGAPPPSK* |
| 436 | HaQGTFTSDYSKYLDARRAKEFIEYLLDGGPSSGAPPPSK* |
| 437 | HaQGTFTSDYAKYLDARRAKEFIEYLLDGGPSSGAPPPSK* |
| 438 | HAibQGTFTSDYSKYLDERAAQAFIEWLLEGGPSSGAPPPSK* |
| 439 | HaQGTFTSDYSKYLDERAAQDFVQYLLDGGPSSGAPPPSK* |
| 440 | HaQGTFTSDYSKYLDERAAQAFIEWLLEGGPSSGAPPPSK* |
| 441 | HAibQGTFTSDYSKYLDARRAKEFVQYLLDGGPSSGAPPPSK* |
| 442 | HAibQGTFTSDYSKYLDARRAKEFIEWLLEGGPSSGAPPPSK* |
| 443 | HAibQGTFTSDYAKYLDARRAKEFVQYLLDGGPSSGAPPPSK* |
| 444 | HAibQGTFTSDYAKYLDARRAKEFIEWLLEGGPSSGAPPPSK* |
| 445 | HaQGTFTSDYSKYLDARRAKEFVQYLLDGGPSSGAPPPSK* |
| 446 | HaQGTFTSDYAKYLDARRAKEFVQYLLDGGPSSGAPPPSK* |
| 447 | HaQGTFTSDYSKYLDARRAKEFIEWLLEGGPSSGAPPPSK* |
| 448 | HaQGTFTSDYAKYLDARRAKEFIEWLLEGGPSSGAPPPSK* |
| 449 | HAibQGTFTSDYSKYLDERAAQAFVQYLLEGGPSSGAPPPSK* |
| 450 | HaQGTFTSDYSKYLDERAAQDFIEWLLDGGPSSGAPPPSK* |
| 451 | HaQGTFTSDYSKYLDERAAQAFVQYLLEGGPSSGAPPPSK* |
| 452 | HAibQGTFTSDYSKYLDARRAKEFIEWLLDGGPSSGAPPPSK* |
| 453 | HAibQGTFTSDYSKYLDARRAKEFVQYLLEGGPSSGAPPPSK* |
| 454 | HAibQGTFTSDYAKYLDARRAKEFIEWLLDGGPSSGAPPPSK* |
| 455 | HAibQGTFTSDYAKYLDARRAKEFVQYLLEGGPSSGAPPPSK* |
| 456 | HaQGTFTSDYSKYLDARRAKEFIEWLLDGGPSSGAPPPSK* |
| 457 | HaQGTFTSDYAKYLDARRAKEFIEWLLDGGPSSGAPPPSK* |
| 458 | HaQGTFTSDYSKYLDARRAKEFVQYLLEGGPSSGAPPPSK* |
| 459 | HaQGTFTSDYAKYLDARRAKEFVQYLLEGGPSSGAPPPSK* |
| 460 | HaEGTFTSDYAKYLDARRAKEFVQWLVNGGPSSGAPPPSK* |
| 461 | HaEGTFTSDYAKYLDARRAKEFIAWLLDGGPSSGAPPPSK* |
| 462 | HaEGTFTSDYAKYLDARRAKEFIEYLVNGGPSSGAPPPSK* |
| 463 | HaEGTFTSDYAKYLDARRAKEFIAYLLEGGPSSGAPPPSK* |
| 464 | HaEGTFTSDYAKYLDARRAKEFIEWLVNGGPSSGAPPPSK* |
| 465 | HaEGTFTSDYAKYLDARRAKEFVQWLKAGGPSSGAPPPSK* |
| 466 | YaEGTFTSDYAIYLDAQAQQDFVQWLLAGGPSSGAPPPSK\hat{ } |
| 467 | YaEGTFTSDYSIYLDKIAQQDFVQWLLAGGPSSGAPPPSK\hat{ } |
| 468 | HAibQGTFTSDYSKYLDERAAQDFVQWLLDGGPSSGAPPPSK* |
| 469 | HAibQGTFTSDYSKYLDKIAAQDFVAYLLDGGPSSGAPPPSK\hat{ } |
| 470 | YaEGTFTSDYSIYLDKIAQQDFVQWLLAGGPSSGAPPPSK* |
| 471 | YAibEGTFTSDYSIAibLDKIAQK\hat{ }AFVQWLIAGGPSSGAPPPS |

With regard to retro-Michael resistance of contemplated fusion proteins presented herein, the inventors unexpectedly discovered that a retro-Michael resistant fusion protein can be prepared where a peptide (most typically a Class B GPCR agonist as shown herein) is attached at the C-terminus to a linker group (typically via an amide bond), which further includes a planar Michael acceptor group as a coupling group (e.g., maleimide or bromomaleimide group) that can then react with the Michael donor group (e.g., thiol group of Cys₃₄ in albumin) to so form a Michael addition conjugate. Suitable linkers will provide sufficient flexibility and steric distance such that the bound agonist peptide will be able to bind (typically in a two-step process) to the GPCR such that the GPCR will be activated.

Therefore, in at least some embodiments, the reactive group (e.g., maleimide group) that couples the linker to the Cys₃₄ thiol group of albumin and the group (e.g., amino group) that couples the linker to the agonist peptide will have a linear distance equivalent to at least 6 carbon-carbon bonds, or at least 8 carbon-carbon bonds, or at least 10 carbon-carbon bonds, or at least 12 carbon-carbon bonds, or at least 14 carbon-carbon bonds, or at least 18 carbon-carbon bonds, or at least 24 carbon-carbon bonds. As will also be readily appreciated, the linker may further include one or more non-carbon atoms to so form an amide bond, an ether bond, etc. Thus, in at least some embodiments preferred linkers will be hydrophilic linkers.

For example, contemplated linkers include AEEA, AEEA-MPA, AEEA-(bromo) MPA, (AEEA) 2-MPA, (AEEA) 2-(bromo) MPA, AEEEA-OA-MPA, AEEEA-OA-(bromo) MPA, AEEA-OA-AEEA-MPA, AEEA-OA-AEEA-(bromo) MPA, (AEEEA) 2-OA-MPA, or (AEEEA) 2-OA-(bromo) MPA in which AEEA denotes (amido (ethoxy) ethoxy acetic acyl), MPA denotes maleimidopropionic acid, and OA denotes 8-aminooctanoate.

In this context, it should be particularly appreciated that upon Michael reaction of a thiol group with a planar maleimide group, absent any stereochemically biasing factors, the reaction product includes a newly generated chiral carbon atom (to which the sulfur atom is covalently bound), which will result in a racemic product. Viewed from a different perspective, a Michael addition reaction product, absent any stereochemically biasing factors, will be an equal mixture of R- and S-configured chiral carbon atoms. Moreover, it should be appreciated that (as with any other chemical reaction) a Michael addition reaction is a reversible reaction unless other factors will hinder such reaction. Indeed, such retro-Michael addition reaction is well known in the context of antibody-drug conjugates where a drug can readily de-conjugate in a retro-Michael addition reaction where the drug was attached to an exposed thiol group.

In view of the above, the inventors have unexpectedly discovered that a peptide agonist with a linker having a maleimide or bromo maleimide coupling group can be coupled to Cys₃₄ of albumin in a stereoselective and retro-Michael resistant manner such that the peptide agonist remains covalently bound to the albumin without decoupling. More specifically, the inventors recognized that as the Cys₃₄ is located in a hydrophobic crevice that is 9.5-10 Å deep in which the walls of the crevice are populated by multiple amino acid side chains, the spatial organization surrounding the Cys₃₄ thiol group can have a substantial impact on the stereo-specificity of the newly created chiral center. Surprisingly, the inventors noticed that the spatial organization of the hydrophobic crevice can be modulated by changing the levels of sodium octanoate (or other fatty acids or hydrophobic ligands) present in the hydrophobic fatty acid binding pockets of albumin during the bioconjugation process. In particular, the inventors observed that where a Michael addition was performed when all or most hydrophobic fatty acid binding pockets were occupied by octanoate or other hydrophobic molecule, the reaction product was substantially a racemic mixture (i.e., a 50/50 mix of R- and S-configured chiral carbon in the conjugate), whereas stereospecificity of the reaction increased with decreasing octanoate levels in the binding pockets. Moreover, this unexpected finding was further accentuated where the pH during the conjugation reaction was below pH 7.0.

Without wishing to be bound by any theory or hypothesis, the inventors contemplate that fatty acid (or other hydrophobic ligand) binding to albumin changes the 3D conformation in such a manner that the thiol group at Cys₃₄ will be preferentially or be even exclusively available for a Michael addition reaction in only one plane of the maleimide ring and as such preferentially or even exclusively produce only one stereoisomer, resulting in a stereoselective chiral product. Moreover, and particularly at a lower pH (i.e., pH<7.0) the resulting micro-environment inside the “Cys₃₄ hydrophobic crevice” becomes prohibitive to the mechanistic steps required to support a retro-Michael reaction. Hence, unanticipatedly, the stereospecificity of the bioconjugation adduct and its corresponding microenvironment in conformationally modified albumin guard against retro-Michael bond Cys₃₄ sulfur-carbon (MPA) cleavage.

With respect to the removal of octanoate or other hydrophobic compounds from albumin it is contemplated that all methods that are able to reduce the octanoate or other hydrophobic compounds without denaturing the albumin are deemed suitable for use herein, and especially preferred methods include charcoal filtration, dialysis, ion exchange chromatography, etc. (see e.g., Biochim Biophys Acta 1970 Nov. 17; 221 (2): 376-8; Nature Communications Materials; (2020) 1:45). As will be readily appreciated, the albumin is preferably human serum albumin, which may be isolated and purified from human serum, or may be recombinantly produced using a bacterial, or more typically, yeast expression system. Thus, albumin contemplated herein may or may not be glycosylated. In this context is should be appreciated that commercially available highly purified natural or recombinant albumin is typically stabilized with exogenously added octanoate or other short or medium-chain fatty acid.

Moreover, it should be appreciated that the albumin can be entirely or only partially defatted such that at least one, or at least two, or at least three, or at least four, or at least five, or at least five, or at least six, or all hydrophobic fatty acid binding pockets of the albumin are free from a hydrophobic ligand to so form the conformationally modified albumin. The suitable degree of defatting will be at least in part determined by the desired degree of a stereoselective reaction product. Thus, and most typically, the albumin will be fully (all hydrophobic fatty acid binding pockets of the albumin are free from a hydrophobic ligand) or almost fully (at least four hydrophobic fatty acid binding pockets of the albumin are free from a hydrophobic ligand) defatted. Once defatted, the so prepared conformationally modified albumin is then reacted with the linker containing the coupling group (typically comprising a maleimide or bromo-maleimide group) following standard Michael addition reaction conditions, preferably at an equimolar ratio, and preferably at a pH that is pH<7.0 (e.g., pH between 6.0 and 6.9, or pH between 5.5 and 6.0, or pH between 5.0 and 5.5, or pH between 4.5 and 5.0, or pH between 4.0 and 4.5). However, excess albumin during the coupling reaction is also deemed suitable, such as a molar excess of 1.1-fold, or 1.2-fold, or 1.3-fold, or 1.4-fold, or 1.5-fold, and even more. Exemplary reaction conditions for coupling the linker to the Cys₃₄ of the conformationally modified albumin are described, for example at Endocrinology (2005) 146 3052-3058, Bioorg. Med. Chem. Lett. (2003) 13 3571-3575, and Bioconjug. Chem. (2005) 16 1000-1008, each of which are incorporated by reference herein. As such, these reactions may be used to reduce or even entirely avoid retro-Michael reactions in albumin with Michael conjugates, particularly where such conjugates have therapeutic use such as with the agonist peptides with linkers and reactive group as listed in SEQ ID NO:1-471.

In yet another unexpected finding, the inventors discovered that even when octanoate levels are at higher levels during conjugation, a retro-Michael resistant fusion protein can nevertheless be generated via a stereoselective translocation to an unreacted albumin having an unreacted Cys₃₄ group (which may or may not be conformationally modified) in a pharmaceutical formulation post standard purification. Alternatively, the conjugation ratio of peptide intermediate with a linker and MPA as a reactive coupling moiety to the albumin can be adjusted such as to provide an excess amount of unreacted albumin having an unreacted Cys₃₄ group (which may or may not be conformationally modified) when the purification step is an ultrafiltration or diafiltration step, leading to a formulation that also contains an excess amount of unreacted albumin.

In view of the above, it should therefore be recognized that albumin can be chemically modified with a coupling group at Cys₃₄ such that a sulfur atom of the Cys₃₄ is covalently bound to a chiral carbon atom of the coupling group, and such that the chiral atom has a favored stereochemical configuration (instead of a racemic carbon atom where the thiol group is not sterically constrained as discussed above). Indeed, it is contemplated (and has been observed) that the favored stereochemical configuration can be favored at a ratio of at least 60:40, or at least 70:30, or at least 80:20, or at least 85:15, or at least 90:10, or at least 95:5, particularly with increasing degree of defatting (i.e., at least one, or at least two, or at least three or at least four, or at least five, or at least six, or all hydrophobic binding pockets are not occupied by a non-covalently bound lipid).

Therefore, it is noted that the inventors discovered that a chemically modified albumin can be produced, where the albumin is modified at Cys₃₄ with a Michael addition conjugate, and where the Michael addition conjugate is resistant to a retro-Michael addition reaction. Indeed, it should be appreciated that it is now possible to prepare compositions in which a coupling group (carrying a (hydrophilic) linker and optionally a peptide coupled to the linker) can be covalently attached to Cys₃₄ of albumin in a stereopreferred (i.e., at least 55%, at least 60%, at least 75%, at least 80%, at least 85%, at least 90%, at least 95% of all chiral carbon atoms in C—S bonds in coupling groups have a configuration that is retro-Michael resistant) or even stereoselective (between 98 and 100% of all chiral carbon atoms in C—S bonds in coupling groups have a configuration that is retro-Michael resistant) manner, thereby rendering the molecular entity attached to the coupling group retro-Michael resistant.

Viewed from a different perspective, in an isotonic buffered (pH 7.0) solution containing contemplated retro-Michael resistant conjugates of albumin and agonist peptide, no more than 40%, or no more than 30%, or no more than 20%, or no more than 15%, or no more than 10%, or no more than 8%, or no more than 6%, or no more than 4%, or no more than 2%, of all bound agonist peptide will dissociate from the albumin in a retro-Michael reaction over a period of 24 hours at room temperature. Therefore, it should be appreciated that the majority of the linkers with covalently bound GPCR agonist peptides will be bound to the Cys₃₄ amino acid of albumin in a retro-Michael resistant manner. Thus, pharmaceutical compositions in which a GPCR agonist peptide is covalently bound to a Cys₃₄ amino acid of albumin in a stereopreferred or stereoselective configuration will be substantially free of unbound GLP-1 agonist peptide (e.g., no more than 10%, or no more than 7%, or no more than 5%, or no more than 3%, or no more than 2% of total agonist peptide in the composition are unbound).

As already noted above, such compositions can be prepared via reacting the coupling group and linker (with or without attached peptide or other molecular entity) to the Cys₃₄ of a conformationally modified albumin, by performing the Michael addition in the presence of free albumin in molar excess relative to the coupling group, and/or by adding free albumin to a Michael addition product that was produced by reacting Cys₃₄ of an albumin with a coupling group and linker (with or without attached peptide or other molecular entity such as a linker-bound GPCR agonist peptide). As will be readily appreciated, the ratio of free albumin to albumin with a ligand bound to Cys₃₄ can vary, and suitable ratios will typically be 1:5, or 1:4, or 1:3, or 1:2, or 1:1.5, or 1:1.2, or 1:1, or 1.2:1, or 1.5:1, or 2:1, or 3:1, or 4:1, or 5:1. In each of the cases where additional albumin is used, such albumin can be conformationally modified and/or conformationally unmodified albumin. Moreover, it should be recognized that once the coupling reaction of the linker (typically also containing the agonist peptide) with the albumin is concluded, the GPCR agonist fusion protein can then be purified using standard methods well known in the art. Moreover, and where desired, octanoate or hydrophobic agents may be added to the fusion protein to re-occupy the hydrophobic binding sites, thereby improving stability of the fusion protein.

Further aspects, examples, and details of synthesis of the compounds presented herein are described in Applicant's International application with the title “Albumin Bound Macromolecule Tri-Agonist Activating GLP-1/GIP/Glucagon Receptors”, filed on or about Jun. 25, 2024, incorporated by reference herein.

Notably, the compounds presented herein exhibited significant biological effects due to the specific binding to and activation of the GPCR such as the GLP-1 receptor, the GIP receptor, and/or the GcG receptor as is shown in more detail below. Moreover, it should be appreciated that by the appropriate choice of amino acids in the agonist peptide, the biological activity could be modulated such as to produce a mono-agonist (e.g., agonist activating only GLP-1 receptor), a dual agonist (e.g., agonist activating GLP-1 and GIP receptors), or a tri-agonist (e.g., agonist activating GLP-1, GIP, GcG receptors). Consequently, it should be appreciated that the compounds presented herein can be tailored to a specific therapeutic profile.

Viewed from a different perspective, the covalent binding of albumin to single or multi-agonist peptides, optionally combined with amino acid sequences which inhibit peptide degradation create therapeutic entities with reduced endocytic degradation intracellularly at any level and enable an order of magnitude higher potency of the agonist peptides compared to currently known peptide agonist compositions. Moreover, the covalent binding of albumin to single or multi-agonist peptides limits the degrees of rotational freedom and thereby enables maximum affinity binding of the agonist peptide to multiple agonist receptors such as the GLP-1 receptor, the GIP receptor, and/or the glucagon receptor, resulting in an increase glycemic control with increased food intake. Still further, HbA1c reduction, weight loss improvement, nausea reduction with increase food intake and balanced blood sugar decrease upon feeding reflect the activation of GLP-1, GIP and Glucagon in terms of each physiological activity.

Moreover, peptides with irreversible binding to albumin will enable transcytosis through the endothelial cell via gp60 receptor and Caveolin-1 to reach beta or alpha cells in the pancreas or small bowel tissue microenvironment, and binding to the various receptors on these cells involved in the incretin and glucose homeostasis and appetite pathway. In addition, irreversible binding also reduces lysosomal degradation and increases half-life via FcRn receptor transcytosis/recycling.

Therefore, it should be appreciated that the compounds and compositions presented herein can be used as pharmaceutical agents that are effective in the treatment of a disease or disorder that is responsive to activation of one or more specific GPCR such as the GLP-1, GIP, and/or GcG receptors. Thus, contemplated diseases or disorders suitable for treatment with contemplated compounds include various metabolic disorders, including NIDDM (non-insulin dependent diabetes mellitus), NASH (non-alcoholic steatohepatosis/steatohepatitis), chronic kidney disease, obesity, cachexia, sarcopenia, diabetic kidney disease, diabetic foot ulcer, and diabetic retinopathy. Moreover, contemplated diseases or disorders suitable for treatment with the compounds presented herein include select neurodegenerative diseases such as Parkinson's Disease, Alzheimer's Disease, peripheral neuropathy, multiple sclerosis, and ALS (amyotrophic lateral sclerosis). Still further contemplated diseases and disorders deemed to be responsive to treatment with contemplated compounds include autoimmune or inflammatory diseases such as Chron's disease, irritable bowel syndrome (IBS), ulcerative colitis (UC), and polycystic ovary syndrome (PCOS).

Moreover, it should be particularly appreciated that the treatments contemplated herein will even include treatments that are otherwise difficult to reach with peptide agonists or other protein-base drugs such as the cerebrovascular space that is typically protected by the blood brain barrier. As contemplated retro-Michael resistant compounds retain the therapeutic ligand bound to the albumin at all times, the ligand can be transported across cell membranes via gp60-mediated transcytosis and/or FcRn recycling. Therefore, target tissues suitable for treatment include all tissues that can import albumin and therefore not only include hepatic tissue, pancreatic tissue, and neural tissue, but also tumor tissues that exhibit higher expression and/or activity of gp60 and/or FcRn.

Contemplated pharmaceutical formulations will contain retro-Michael resistant fusion proteins presented herein in an amount such as to allow administration of between about 1 mg and 15 mg of the retro-Michael resistant fusion protein, equating to about 20 mcg and 1,000 mcg of the peptide agonist, preferably in a single dose. Therefore, suitable pharmaceutical formulations will typically contain the retro-Michael resistant fusion protein at a concentration of between 1-2 mg/mL, or 2-5 mg/mL, or 5-7.5 mg/mL, or 7.5-10 mg/mL, or 10-12 mg/mL, or 12-15 mg/mL, or 15-17 mg/mL, or 17-20 mg/mL, or 20-22 mg/mL, and in some cases even higher. Viewed from a different perspective, contemplated pharmaceutical formulations will contain the peptide agonist at a concentration of between 20-60 mcg/mL, or between 50-150 mcg/mL, or between 100-300 mcg/mL, or between 250-500 mcg/mL, or between 300-700 mcg/mL, or between 700 and 1,400 mcg/mL. Consequently, it should be noted that therapeutically effective doses can be administered in a single injection (e.g., subcutaneous) at a volume of equal or less than 5 mL, or equal or less than 2.5 mL, or equal or less than 1 mL, or equal or less than 0.75 mL, or equal or less than 0.5 mL, or equal or less than 0.25 mL, or equal or less than 0.1 mL, or equal or less than 0.075 mL, and even less.

Thus, it should also be recognized that the pharmaceutical formulations contemplated herein will be formulated for administration once weekly, but different schedules also contemplated such as twice weekly, four times weekly, and even daily. However, administration is more preferably no more than once weekly, and in some embodiments every 10 days, every other week, once monthly, etc. Still further, it should be appreciated that contemplated pharmaceutical compositions can be administered without the need for titration, particularly where the dosage administered is at or below 200 mcg of agonist peptide. However, titration to the therapeutically desired dosage is also contemplated and may include titration over at least 1 week or at least 2 weeks, or at least 3 weeks, or at least 4 weeks, or even longer. Moreover, it should be noted that the treatment using contemplated compositions may accompanied by other pharmaceutical treatments such as treatment with another blood glucose regulatory agent, including metformin or a thiazolidinedione drug.

Most preferably, contemplated pharmaceutical formulations will have a pH that is less than pH<7.0, such as pH<6.8, or pH<6.6, or pH<6.4, or pH<6.2. However, in preferred embodiments the pH will be between pH 4.0 and 6.0, such as between 4.0 and 4.4, or between 4.4 and 4.8, or between 4.8 and 5.2, or between 5.2 and 5.6, or between 5.6 and 6.0. Moreover, it is contemplated that the pharmaceutical formulation will typically include a buffer to maintain the pH, and exemplary buffers include a phosphate buffer, a citrate buffer, an acetate buffer, a succinate buffer, a histidine buffer, a glycine buffer, etc. In most embodiments, it is contemplated that the buffer strength will be at a strength of equal or less than 100 mM, or equal or less than 75 mM, or equal or less than 50 mM, or equal or less than 25 mM, or equal or less than 10 mM, or even less, but more than 1 mM. For example, suitable buffer strengths will be between 10-50 mM, or between 25-75 mM, or between 50-100 mM.

Furthermore, it should be appreciated that the formulations herein will be isotonic formulations containing a tonicity agent such as sodium chloride, calcium chloride, magnesium chloride, lactose, sorbitol, sucrose, mannitol, trehalose, raffinose, polyethylene glycol, hydroxyethyl starch, glycine, etc. Where desired, one or more surfactants may be included to reduce protein aggregation, control droplet size for spray formulations, etc., and contemplated surfactants include polysorbates (e.g. polysorbates 20 or 80); poloxamers (e.g. poloxamer 188 (pluronic F68)); Triton; sodium dodecyl sulfate (SDS); sodium laurel sulfate; sodium octyl glycoside, etc. In addition, and especially where the formulation is packed for multiple administrations, preservatives are also contemplated and include m-cresol, benzyl alcohol, methanol, ethanol, iso-propanol, butyl paraben, ethyl paraben, methyl paraben, phenol, glycerol, xylitol, resorcinol, cathechol, 2, 6-dimethylcyclohexanol, 2-methyl-2,4-pentadiol, dextran, polyvinylpyrrolidone, 2-chlorophenol, benzethonium chloride, merthiolate (thimersosal), benzoic acid, etc.

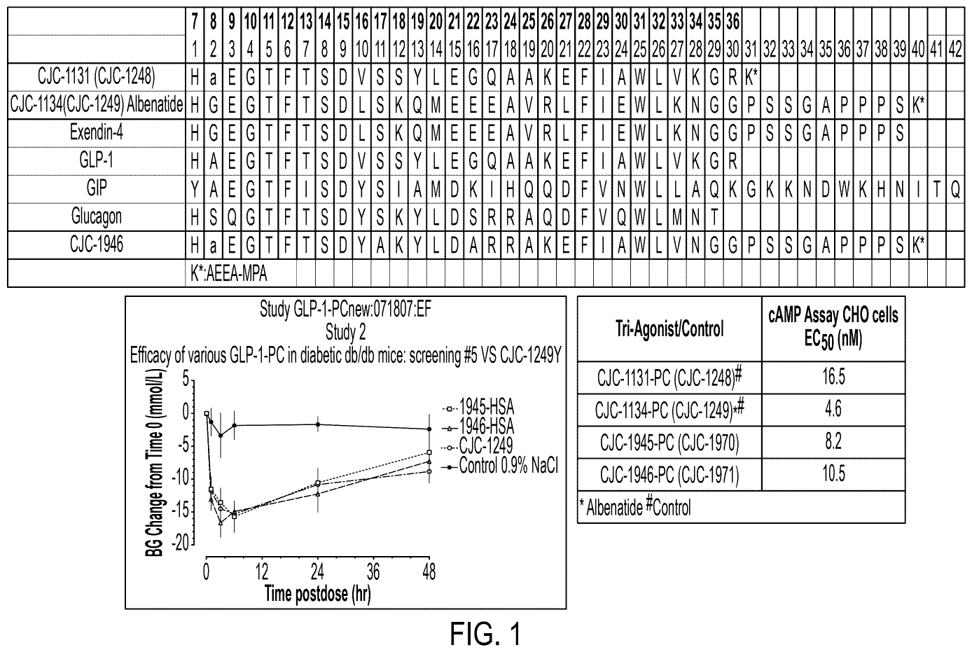

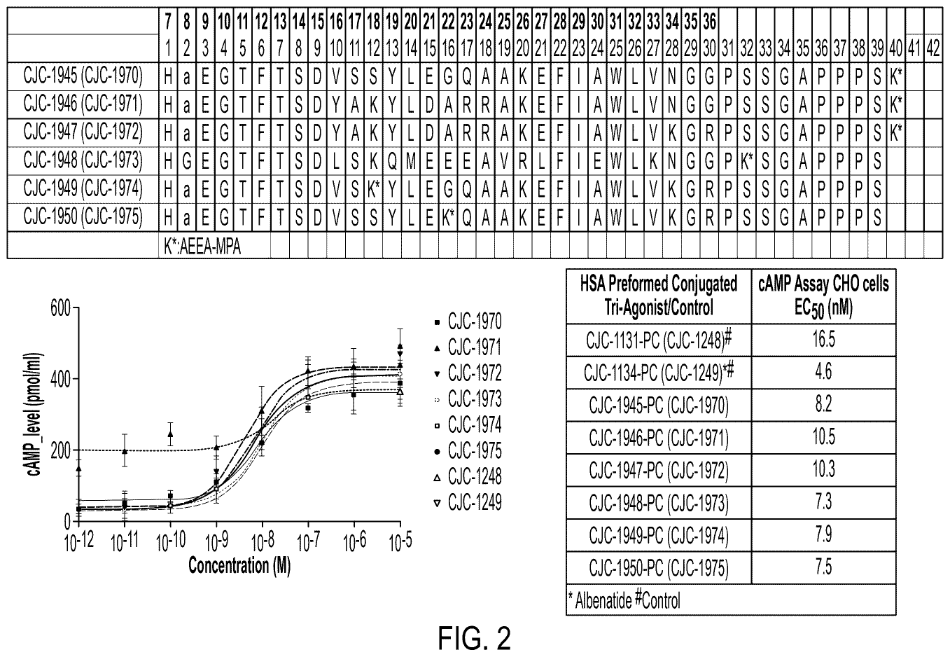

To demonstrate the in vitro and in vivo activity of the compounds presented herein, the inventors performed numerous experiments using selected compounds in vitro with CHO cells quantitating EC₅₀ for the tested compounds following protocols well known in the art (e.g., using cAMP Hunter CHO-K1 GLPIR Gs Cell Line, EUROFINS, Cat No: 95-0062C2), and diabetic db/db mice. Here, FIGS. 1-11 provide exemplary in vitro and in vivo data for selected retro-Michael resistant fusion proteins according to the inventive subject matter, demonstrating that the compounds presented herein can be tailored to exhibit GLP-1, GIP, and/or GcG mediated activity. For example, FIG. 1 depicts exemplary results for retro-Michael resistant albenatide (AB-013-AEEA-succinimide-albumin) CJC-1249 compared to other retro-Michael resistant albumin conjugates. As can be readily seen from the data in FIG. 1, Michael resistant albenatide and the other exemplary conjugates not only had nanomolar EC₅₀ values but also exhibited biological activity in diabetic mice reducing blood glucose over at least 48 hours. Such long reduction is indicative of substantially increased half-life of the tested retro-Michael resistant albumin conjugates while retaining substantial binding and glycemic control in vivo. Similarly, FIG. 2 depicts exemplary results for retro-Michael resistant albenatide (AB-013-AEEA-succinimide-albumin) (CJC-1249) compared to other retro-Michael resistant albumin conjugates. As can be once more seen from the data in FIG. 2, Michael resistant albenatide and the other exemplary conjugates had nanomolar EC₅₀ values with effective CAMP signaling. Here, it should be noted that the agonist peptide in some of the conjugates was covalently anchored to albumin via an AEEA linker at the C-terminus of the agonist peptide, while other conjugates were attached to the albumin via an AEEA linker at an intermediate position of the agonist peptide.

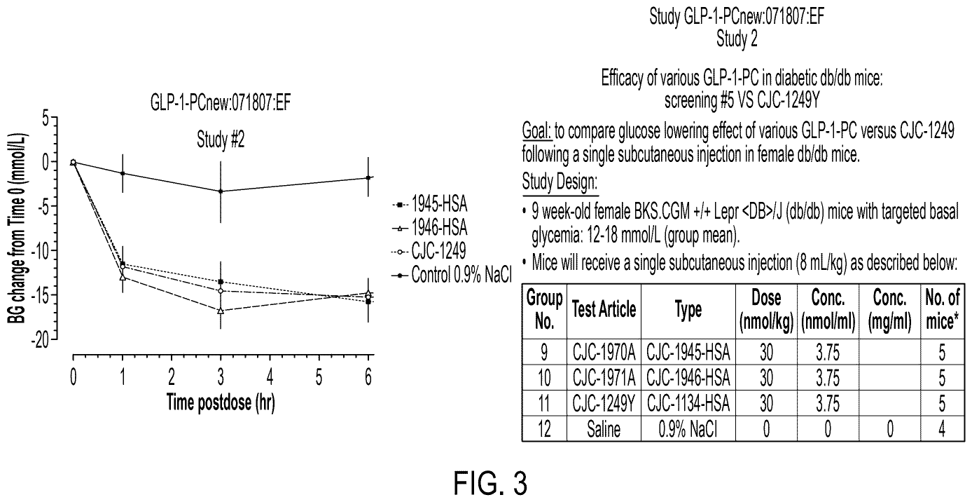

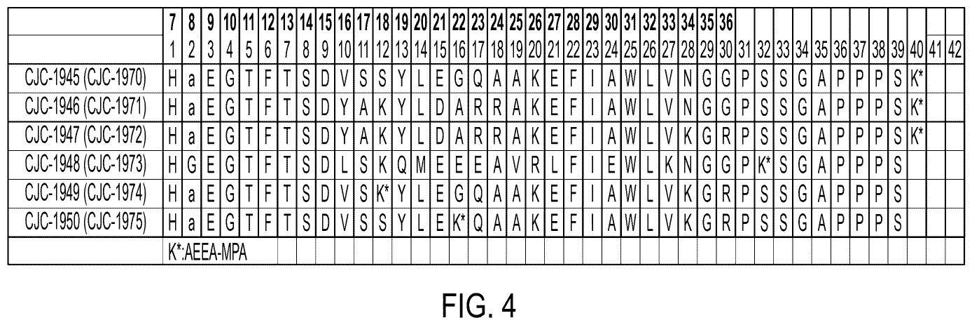

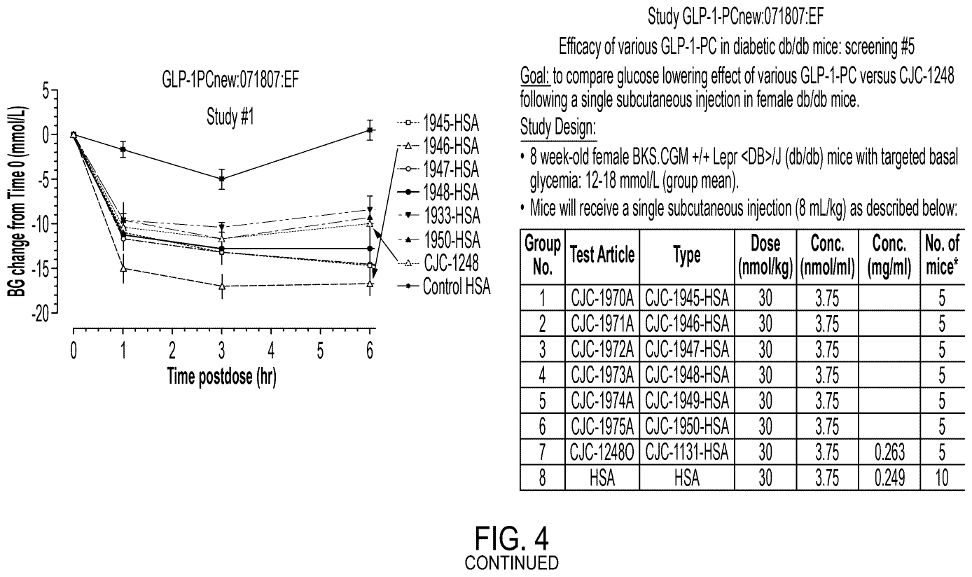

Biological activity was once more tested in vivo using diabetic db/db mice that received a single subcutaneous injection of various tested retro-Michael resistant albumin conjugates (including retro-Michael resistant albenatide) at a dose of 30 nmol/kg. Once more, as can be seen form the exemplary results in FIG. 3, all tested retro-Michael resistant albumin conjugates exhibited significant of blood glucose post injection compared to saline control. FIG. 4 provides additional exemplary data for blood glucose control in db/db mice using various retro-Michael resistant albumin conjugates. Similar to FIG. 2, it should be noted that the agonist peptide in some of the conjugates was covalently anchored to albumin via an AEEA linker at the C-terminus of the agonist peptide, while other conjugates were attached to the albumin via an AEEA linker at an intermediate position of the agonist peptide.

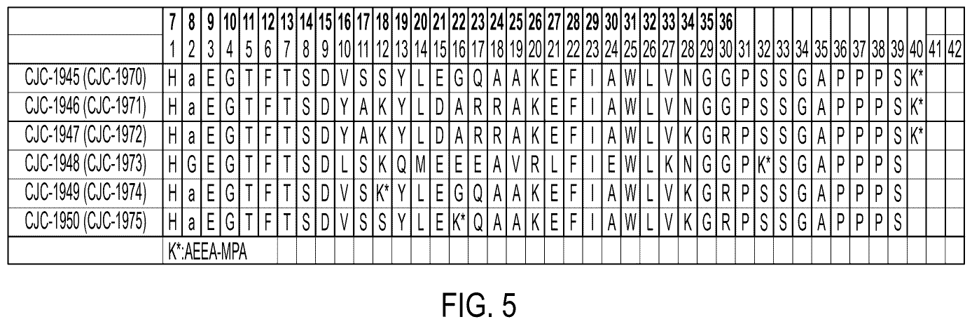

In still further experiments, the inventors tested whether different amino acid modifications would result in different biological potencies. To that end, biological activity was once more tested in vivo using diabetic db/db mice that received a single subcutaneous injection of various retro-Michael resistant tri-agonist albumin conjugates at a dose of 30 nmol/kg. As can be seen from FIG. 5, while all tested compounds other than blank human serum albumin control (albumin without agonist peptide) exerted significant reduction in blood glucose, some of the compounds were more effective than others over the tested period of 6 hours.

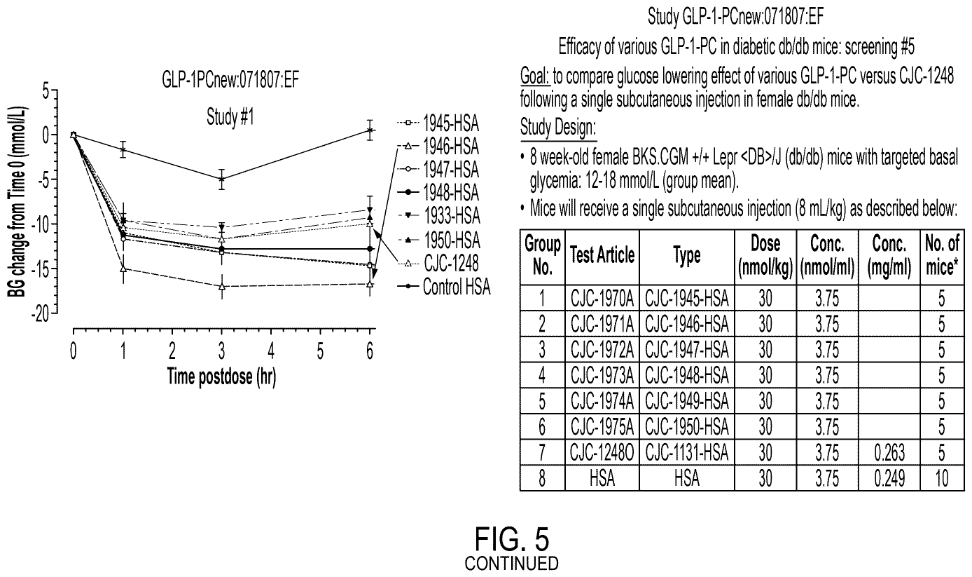

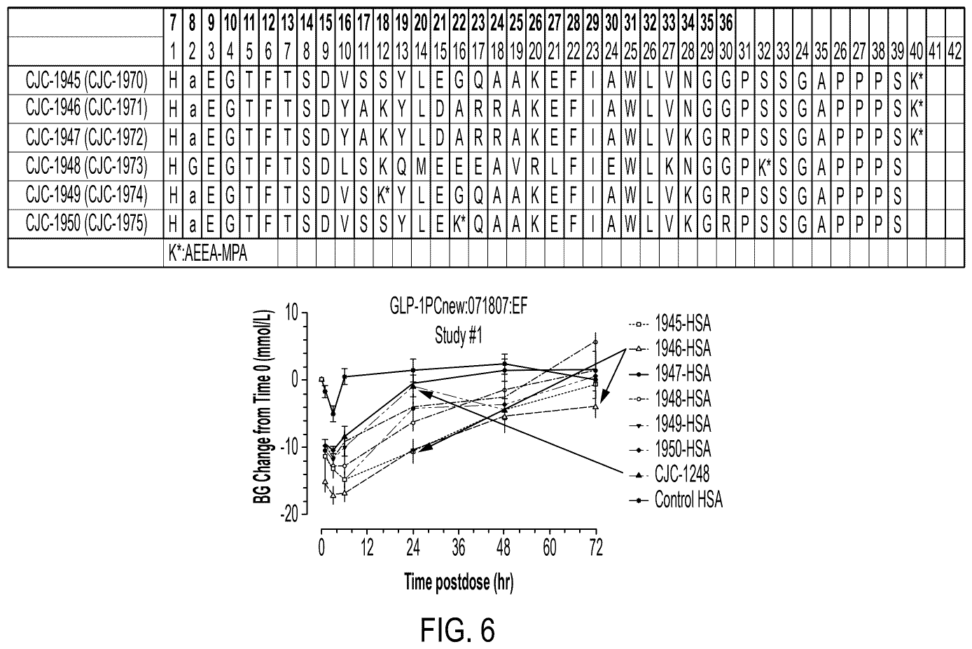

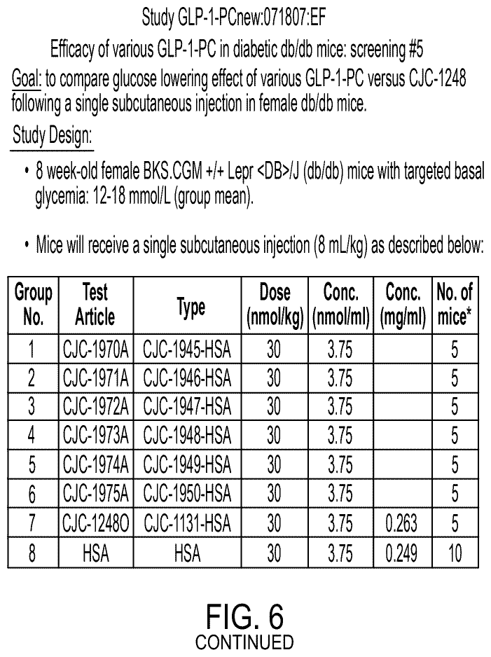

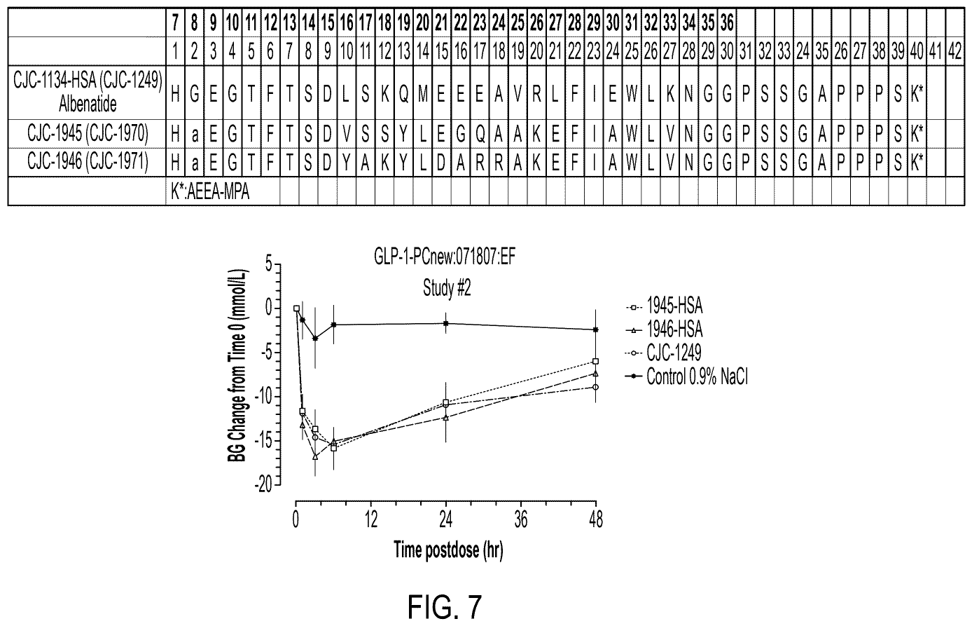

To investigate whether or not the biological effects of the retro-Michael resistant albumin tri-agonist conjugates would persist over an extended period (e.g., at least 24 hours, or at least 48 hours, or at least 72 hours), the inventors tested blood glucose reduction in vivo using diabetic db/db mice that received a single subcutaneous injection of various tested retro-Michael resistant albumin conjugates (including retro-Michael resistant albenatide) at a dose of 30 nmol/kg. Exemplary test data are shown in FIG. 6. Here, it is readily apparent that for most tested compounds the blood glucose reduction persisted through 48 hours, and lasted out to even 72 hours for some of the tested compounds. Notably, the tested retro-Michael resistant albumin tri-agonist conjugates had comparable half-life with regard to blood glucose changes as compared with the retro-Michael resistant (GLP-1 mono agonist) albenatide (AB-013-AEEA-succinimide-albumin) (CJC-1249) as shown in the exemplary data of FIG. 7.

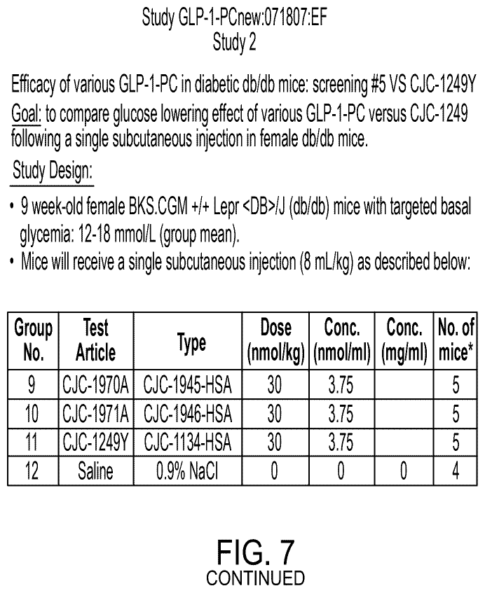

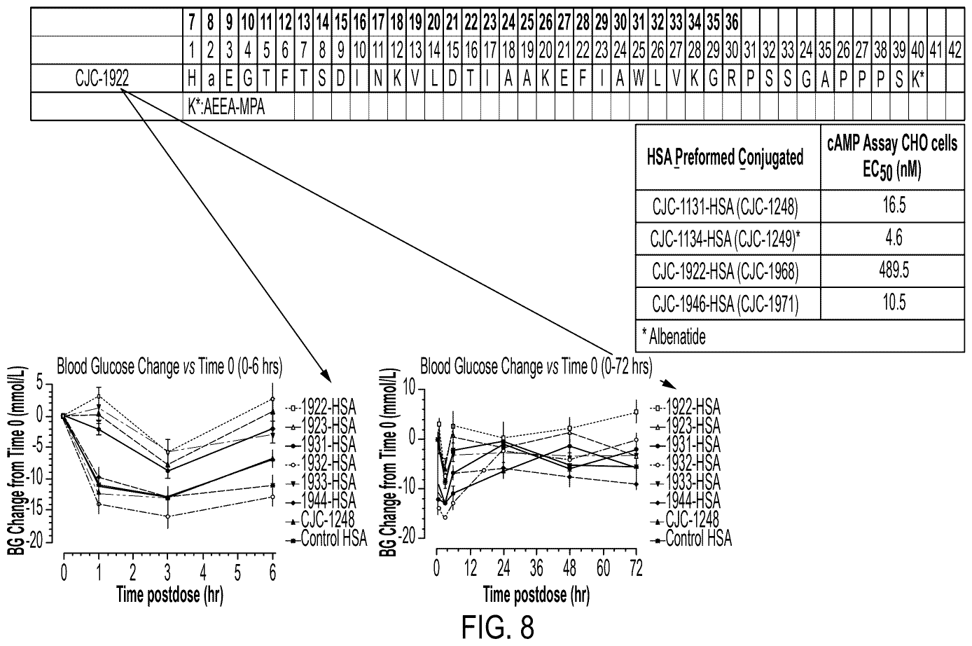

Despite the apparent possibility that numerous amino acids can be modified in the GPCR agonist peptides, the inventors noted that specific amino acid sequences are indeed necessary for binding affinity and biological activity (here: blood glucose reduction). For example, and as is shown in FIG. 8, relatively small changes in the amino acid sequence (relative to the retro-Michael resistant albenatide) led to a dramatic increase of EC₅₀ and substantially abrogated biological activity in an acute (6 hour) and long-term (72 hr) study in retro-Michael resistant CJC-1968.

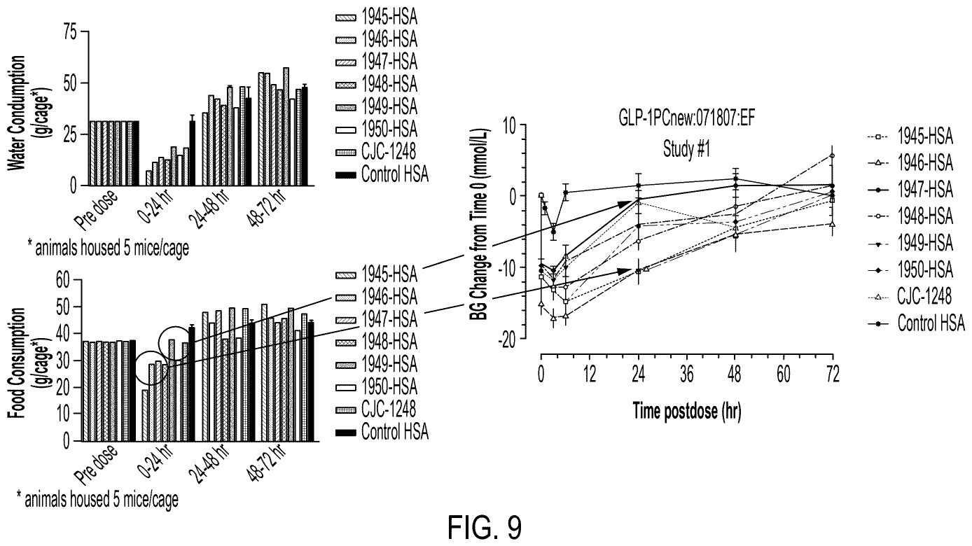

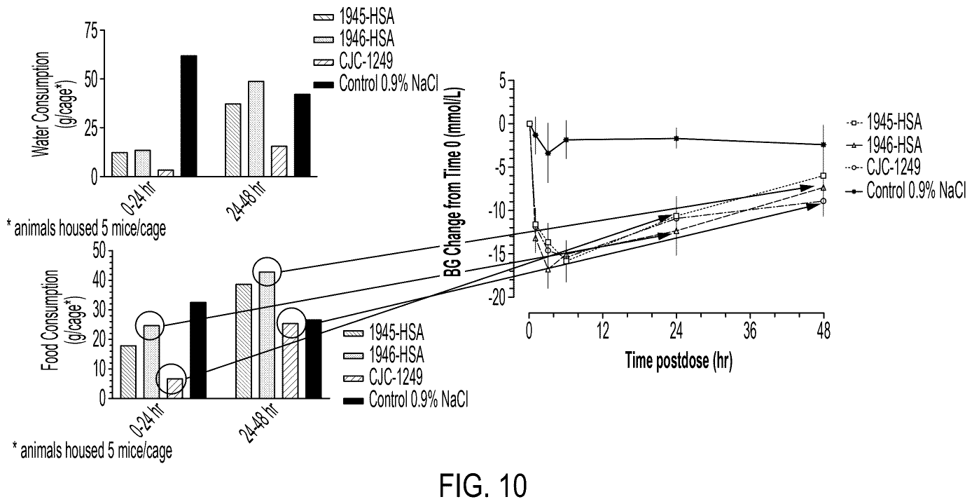

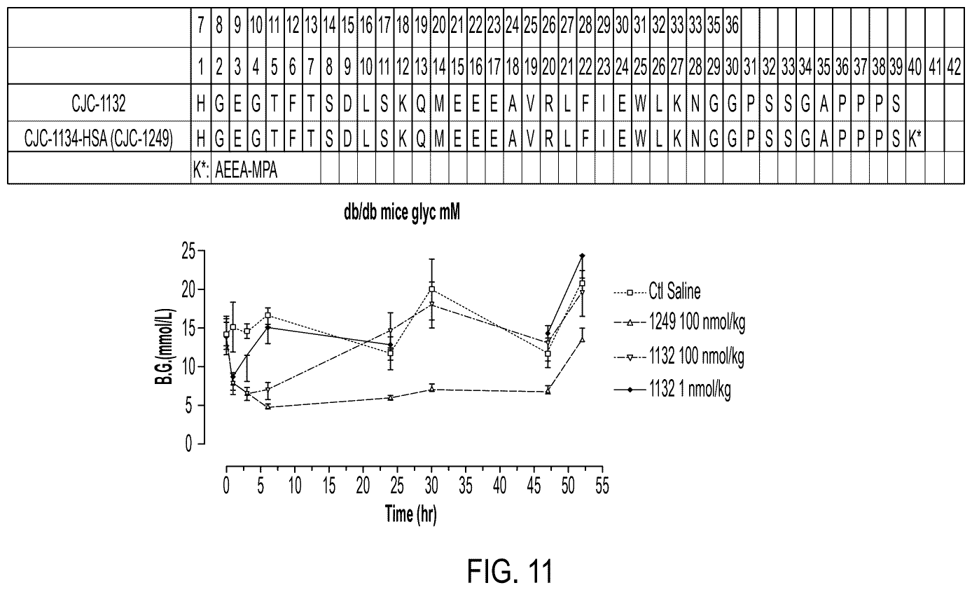

To further explore the physiological effects of various retro-Michael resistant fusion proteins with a tri-agonist peptide agonist, the inventors measured water and food consumption along with blood glucose in the db/db mouse model after a single subcutaneous injection of 30 nmol/kg of the tested fusion proteins. As can be taken from FIG. 9, and consistent with earlier results, the blood glucose was reduced over at least 48 hours while food and water intake was reduced in most cases over at least 24 hours. FIG. 10 depicts further comparative results for selected tri-agonist fusion proteins and retro-Michael resistant (GLP-1 mono agonist) albenatide (AB-013-AEEA-succinimide-albumin) (CJC-1249) in the db/db mouse model. FIG. 11 is a further exemplary result from an in vivo db/db mouse model illustrating the unexpected and significant extension of half-life of the retro-Michael resistant (GLP-1 mono agonist) albenatide (AB-013-AEEA-succinimide-albumin) (CJC-1249) as compared to the corresponding unbound peptide agonist CJC-1132. As can be readily seen from the data, the retro-Michael resistant albenatide fusion protein had extended glucose reducing effects beyond 48 hours, whereas the corresponding unbound agonist peptide had no activity over control in as little as 20 hours. Moreover, it should be noted that the extended activity of the retro-Michael resistant albenatide (AB-013-AEEA-succinimide-albumin) was pronounced, even though the agonist peptide was irreversibly and covalently bound to albumin via a linker. Such activity was entirely contrary to conventional wisdom based on the experience with other fusion proteins (e.g., albiglutide).

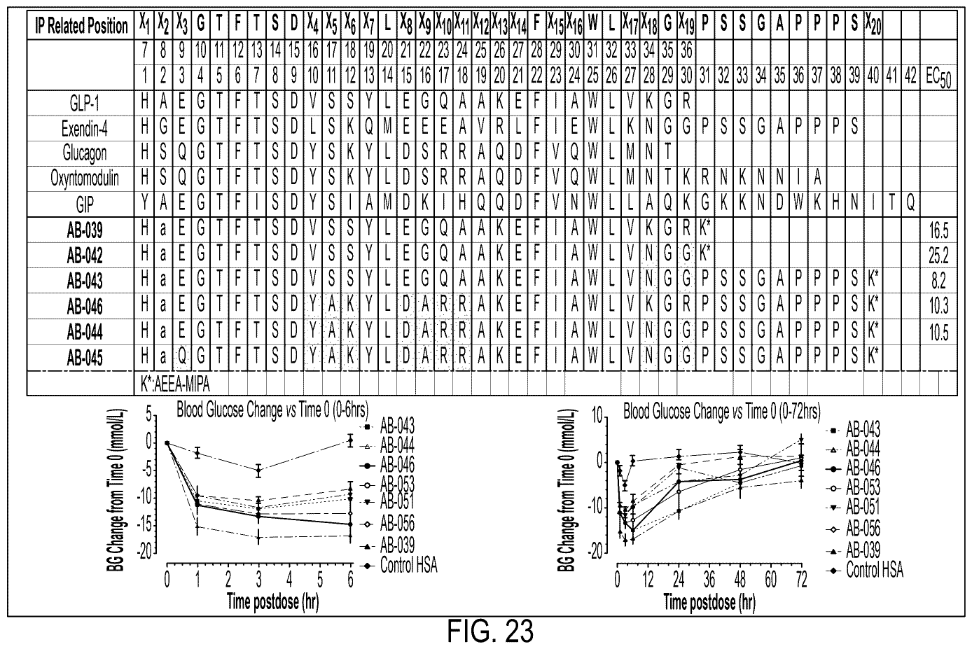

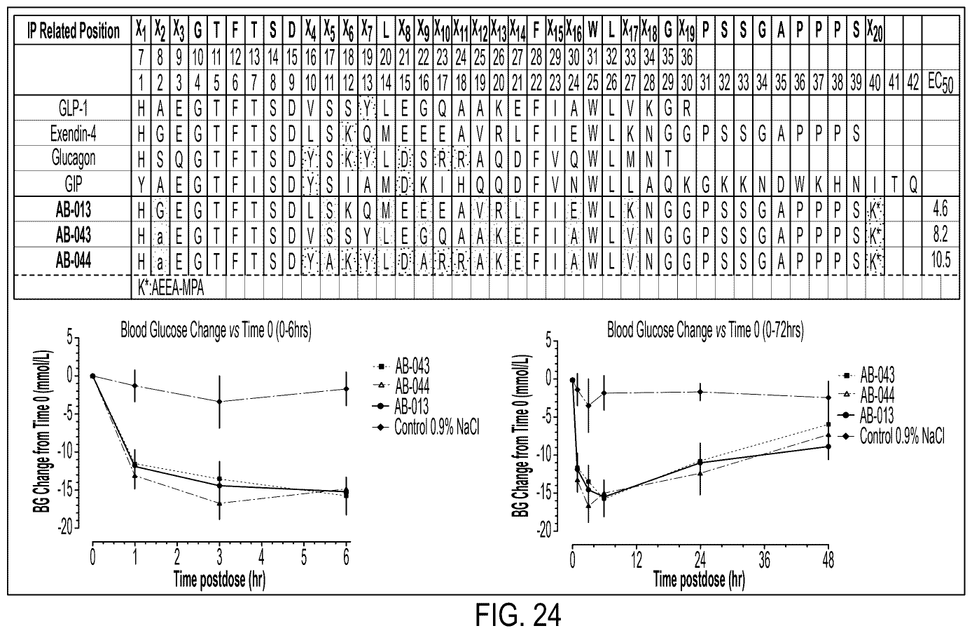

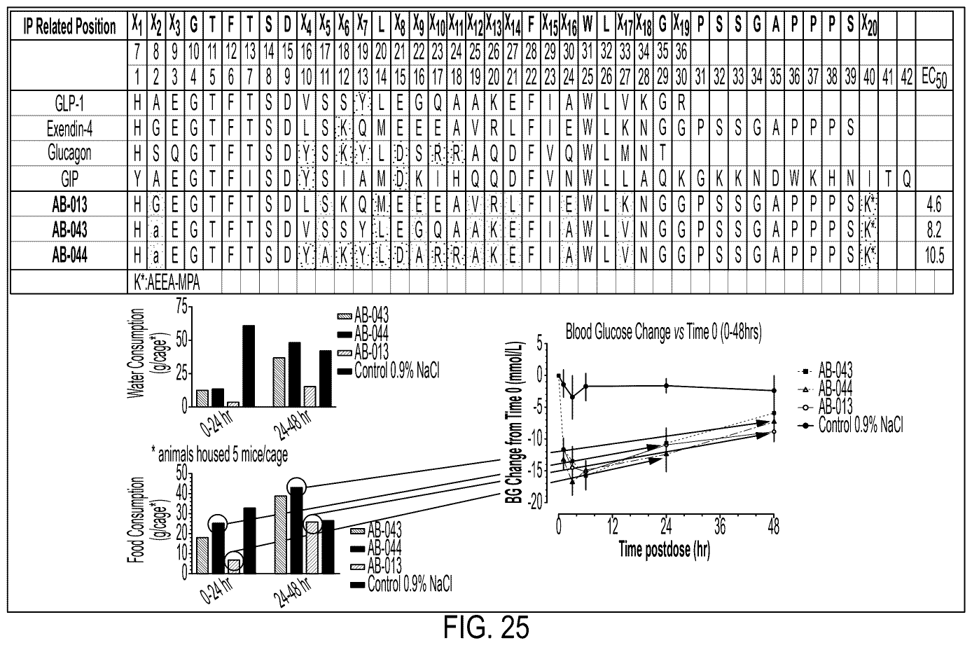

In yet further experiments, the inventors conducted further in vivo studies to test the biological activity of additional retro-Michael resistant fusion proteins according to the inventive subject matter. Unless noted otherwise, the in vivo model used db/db mice, and each test article was administered once by subcutaneous injection. Blood glucose was then measured over the indicated times and exemplary results are depicted in FIGS. 23-31. More specifically, FIG. 23 shows acute (6 hrs) and long-term (72 hrs) changes in blood glucose after administration of the indicated compounds, while FIG. 24 depicts comparative results for changes in blood glucose between the retro-Michael resistant albenatide (AB-013-AEEA-succinimide-albumin) and selected compounds of FIG. 23 over 6 hrs and 48 hours. FIG. 25 depicts further exemplary results for the compounds tested in FIG. 24 with respect to food and water consumption.

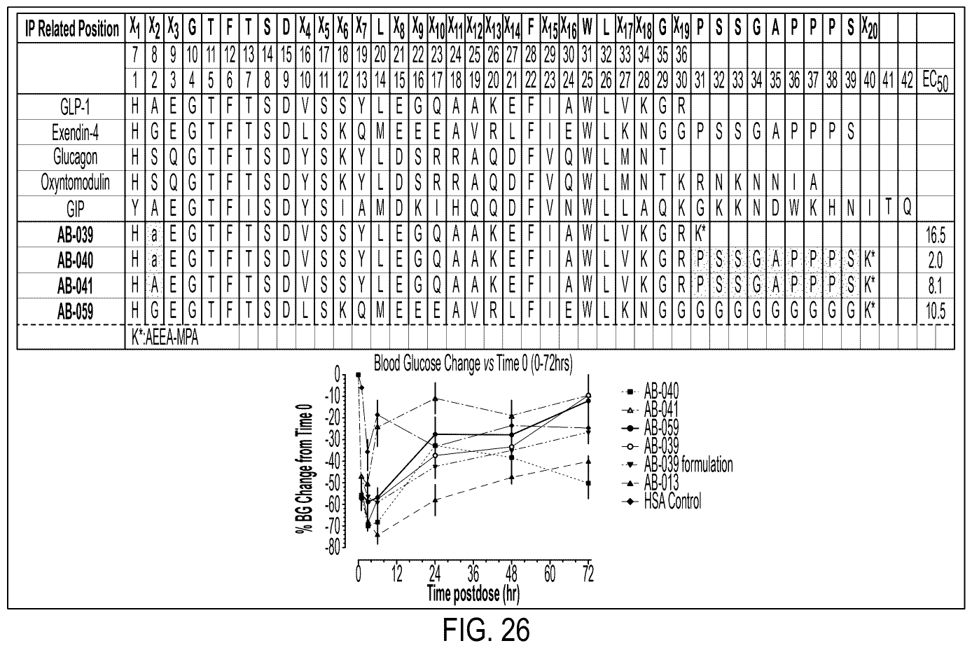

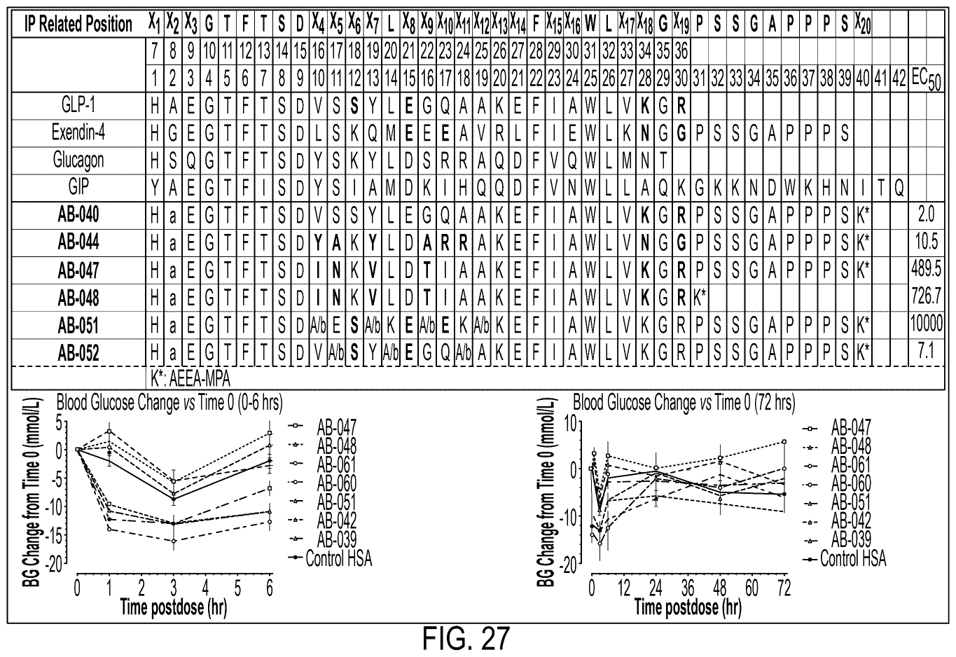

In FIG. 26, still further retro-Michael resistant fusion proteins according to the inventive subject matter were tested with regard to their effect on blood glucose and compared to the retro-Michael resistant albenatide (AB-013-AEEA-succinimide-albumin) over a period of 72 hours. As can be readily taken from the graphs, the retro-Michael resistant albenatide (AB-013-AEEA-succinimide-albumin) had the most potent in vivo effect over the entire test cycle. On the other hand, other tested retro-Michael resistant fusion proteins had limited or even no effect in acute and long-term observations for blood glucose as is shown in FIG. 27.

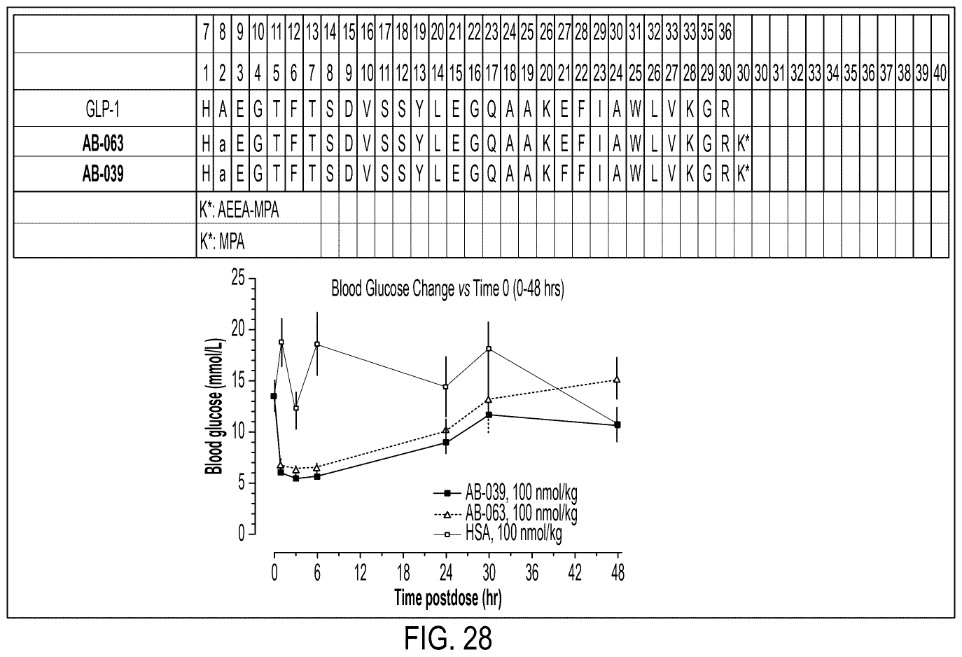

The effect of the linker/coupling group was investigated for a specific peptide agonist sequence where one sequence had only MPA as a linker/coupling group, while the other peptide agonist sequence had an AEEA-MPA linker/coupling group. Notably, as shown in FIG. 28, both tested peptide agonis had significant activity in reducing blood glucose. However, the agonist peptide with the MPA group was over time less effective as the agonist with the AEEA-MPA group.

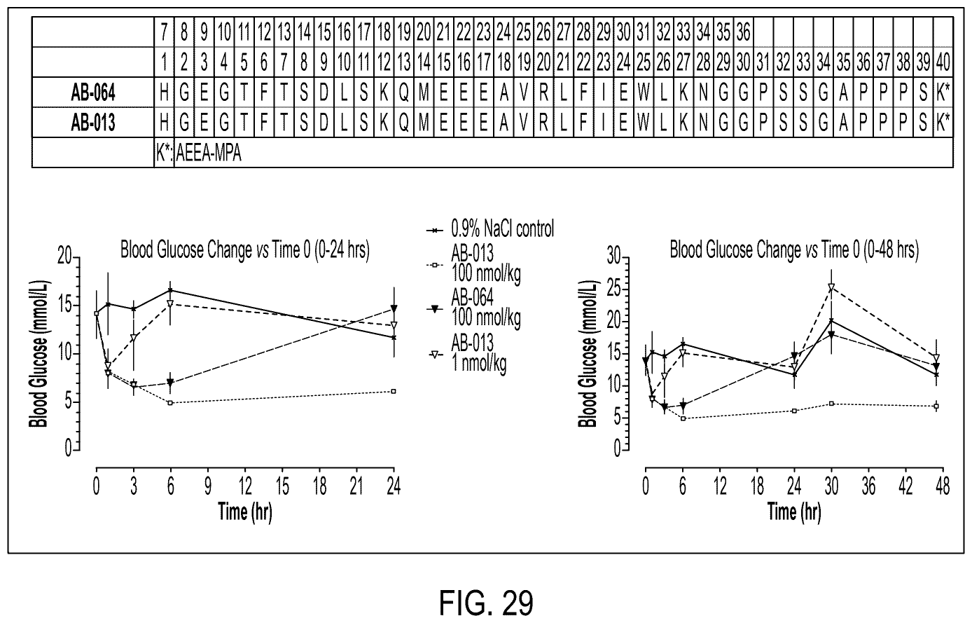

In yet further experiments, the retro-Michael resistant albenatide (AB-013-AEEA-succinimide-albumin) was tested at 1 nmol/kg and 100 mmol/kg and compared to 100 nmol/kg of another retro-Michael resistant fusion protein, AB-064. Interestingly, even at the minute dose of 1 nmol/kg, the retro-Michael resistant albenatide (AB-013-AEEA-succinimide-albumin) still had a measurable effect on blood glucose in the short term, and a sustained substantial effect over 48 hr whereas AB-064 returned to baseline after only 24 hrs as is shown in the exemplary results of FIG. 29.

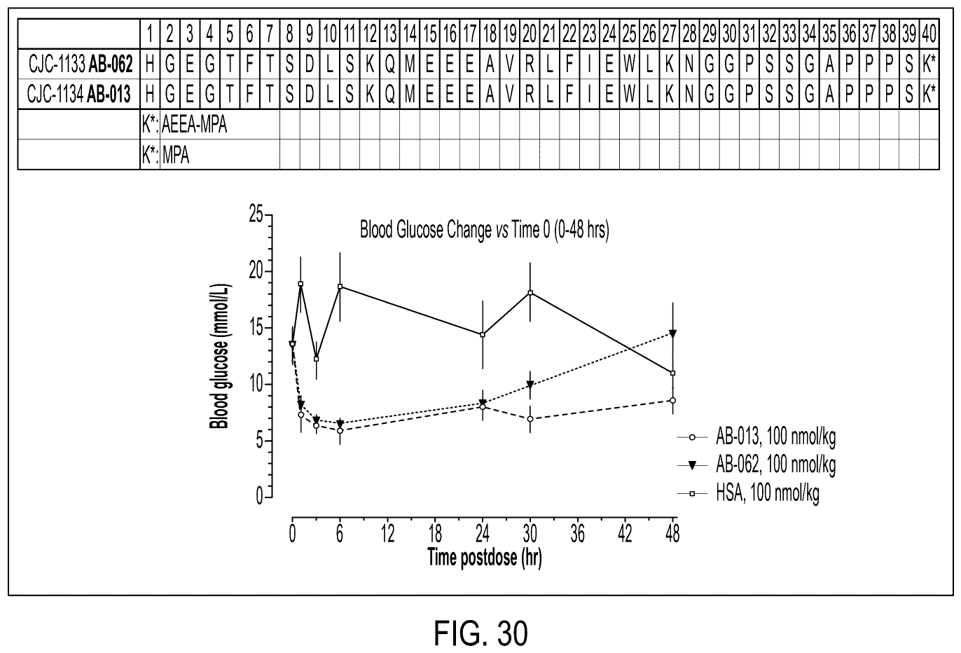

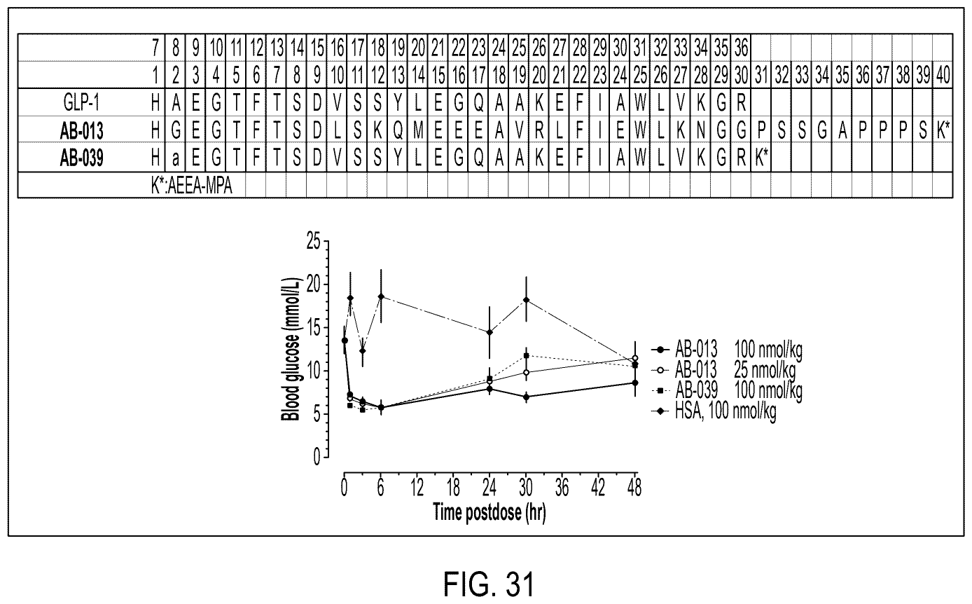

Higher dose administration of 100 nmol/kg of retro-Michael resistant albenatide (AB-013-AEEA-succinimide-albumin) and another retro-Michael resistant fusion protein (AB-062) was evaluated over 48 hours. Notably, and as can be taken from FIG. 30, while AB-062 significantly reduced blood glucose in this mouse model over 24 hours, the reduction in blood glucose was lost at 48 hours. In contrast, administration of retro-Michael resistant albenatide resulted in s sustained reduction in blood glucose beyond the 48 hour time point. Additionally, the inventors tested the retro-Michael resistant albenatide (AB-013-AEEA-succinimide-albumin) at 100 nmol/kg and 25 nmol/kg, and another retro-Michael resistant fusion protein (AB-039) at 100 nmol/kg, and exemplary results are depicted in FIG. 31. As can be readily seen from the graphs, the 100 nmol/kg dose of retro-Michael resistant albenatide (AB-013-AEEA-succinimide-albumin) had sustained effect at 48 hrs, while the 25 nmol/kg dose was still effective at 30 hours.

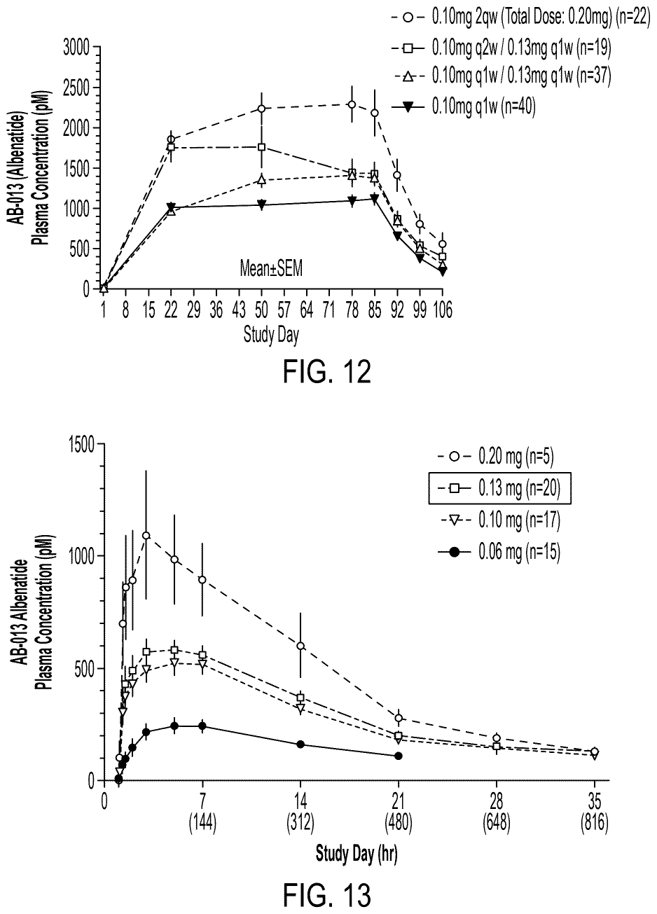

Based on the encouraging data from the in vitro and in vivo experiments, the inventors then set out to determine various pharmacokinetic parameters for retro-Michael resistant albenatide (AB-013-AEEA-succinimide-albumin) in human, and exemplary results are depicted in FIGS. 12-14. In particular, the inventors conducted a dose-finding, randomized, double-blind comparison of retro-Michael resistant albenatide (AB-013-AEEA-succinimide-albumin) versus placebo. Escalating single doses of 0.31, 0.62, 1.25, 2.5, 5.0, and 3.75 mg were administered to dosing cohorts of seven T2DM participants each (6:1 active drug to placebo), and a final cohort of 16 participants (12:4 active drug to placebo) was administered a single dose of 3.0 mg. No dose-limiting toxicities were observed at all doses tested (0.31 to 5.0 mg), and the maximum dose administered (5.0 mg) was considered to be the maximum tolerated dose for this study. In this context it should be appreciated that these doses include the mass of albumin. The dose of 0.31 mg to 5.0 mg translates to 0.02 mg to 0.33 mg of modified Exendin-4 (active peptide). It should also be noted that the study was performed without a 4-week dose escalation titration. All doses were well tolerated.

The inventors then conducted a multiple-dose study conducted in 70 participants with T2DM. This study was a randomized, double-blind comparison of 5 weekly injections of retro-Michael resistant albenatide (AB-013-AEEA-succinimide-albumin) versus placebo, with participants evenly randomized into each of 4 parallel treatment groups: 1.0 mg, 2.0 mg, or 3.0 mg of retro-Michael resistant albenatide (AB-013-AEEA-succinimide-albumin) or placebo. Retro-Michael resistant albenatide (AB-013-AEEA-succinimide-albumin) was well tolerated by participants, and the most common adverse event (AE) that was reported was headache (15 of 52 participants or 17% in the treated groups versus 2 of 18 participants or 11% in the placebo group). Nausea occurred in 17% of participants in the placebo group and 21% of participants in all 3 treatment groups combined (1.0 mg, 2.0 mg, and 3.0 mg). Although vomiting is a common AE associated with GLP-1 agonists, participants in the 1.0- and 2.0 mg groups reported no cases of vomiting, while 5 of 17 (30%) participants in the 3.0 mg group reported at least 1 case of vomiting. No injection-related skin reactions (a 30-gauge needle was used) were reported in the 2.0- or 3.0 mg groups, whereas the placebo group reported 4 skin reactions and the 1.0 mg group reported 1 skin reaction. Furthermore, 21% of participants were found to have low-titer antibodies to retro-Michael resistant albenatide (AB-013-AEEA-succinimide-albumin), but these antibodies had no apparent effects on efficacy or tolerability. No deaths were reported during the study, and 2 serious AEs (SAEs) occurred that were not considered related to study drug.

A second study with a purified composition of retro-Michael resistant albenatide (AB-013-AEEA-succinimide-albumin) was performed with the administration of single escalating-doses of study drug versus placebo to cohorts of 7 participants (6:1 active drug to placebo) followed by larger cohorts who demonstrated tolerability of further exploratory doses (16 to 24 participants randomized 3:1 active drug to placebo). No SAEs or deaths were reported in this study, and there were no clinically relevant changes in safety laboratory parameters and electrocardiograms (ECGs).

Retro-Michael resistant albenatide (AB-013-AEEA-succinimide-albumin) was generally well tolerated at the clinically relevant doses of 1.0 mg, 1.5 mg, and 2.0 mg. The incidence of these AEs in the 1.0- and 1.5 mg groups was similar to that observed in the placebo group. Of the participants in the placebo group, 2 of 26 (8%) reported nausea and 3 of 26 (12%) reported vomiting. When administered at a single dose of 1.0 mg, 2 of 18 (11%) participants experienced either mild nausea or vomiting. When this dose was increased to 1.5 mg, 2 of 18 (11%) participants experienced mild nausea and 1 of 18 (6%) participants reported mild vomiting after dosing. At 2.0 mg, approximately 8 of 23 (35%) and 6 of 23 (26%) participants experienced mild to moderate nausea or vomiting, respectively. Following the administration of 3.0 mg of study drug, 3 of 6 (50%) participants experienced nausea and/or vomiting. The majority of AEs were mild to moderate in severity. No study drug-related injection site reactions were observed. Low-titer antibodies (1:1 to 1:10) specific to retro-Michael resistant albenatide (AB-013-AEEA-succinimide-albumin) were detected in a small number of participants (6%) after a single administration of the therapeutically relevant doses of 1.0 to 3.0 mg. Once more, it should be noted that these doses included the mass of albumin and the study was performed without 4-week dose escalation titration. The dose of 1.0 mg to 3.0 mg translates to 0.065 mg to 0.195 mg of modified Exendin-4 (active peptide agonist).

Data from the 3-month weekly dosing study showed plasma concentrations of retro-Michael resistant albenatide (AB-013-AEEA-succinimide-albumin) increased with weekly administration and appeared to reach a steady state in a dose-proportional manner. The mean steady-state observed predose concentration (Cpredose) value on Day 78 for the antibody-negative participants in the weekly 1.5/2.0 mg (0.10/0.13 mg Ex4) dose group was 33% higher than the mean Cpredose value for the antibody-negative participants in the weekly 1.5 mg (0.10 mg Ex4) dose group in the PP population, indicating a dose-proportional increase in exposure between the 2 dose levels.

As was expected form the mouse models, after the last dose on Day 78, retro-Michael resistant albenatide (AB-013-AEEA-succinimide-albumin) levels decreased very slowly. The mean (coefficient of variation [CV]) half-life (t1/2) values of retro-Michael resistant albenatide (AB-013-AEEA-succinimide-albumin) in the 1.5 mg (0.10 mg Ex4) and 1.5/2.0 mg (0.10/0.13 mg Ex4) dose groups were 9.16 (20) days and 9.42 (24) days, respectively, for all participants in the PP population. The mean (CV) t1/2 values of AB-013 in the 1.5 mg (0.10 mg Ex4) and 1.5/2.0 mg (0.10/0.13 mg Ex4) dose groups were 9.19 (20) days and 9.01 (21) days, respectively, for antibody negative participants in the PP population.

As can be seen from FIG. 12, a dose response effect was observed from 0.1 mg q weekly to 0.13 mg q weekly to 0.20 mg q weekly with peak and steady state plasma concentration levels of AB-013 reaching>2,000 picomole of unbound and fully bound agonist. Once more, it is noted that the dose indicated above included the mass of albumin and the study was performed without q 4-week dose escalation titration. The doses of 1.5 mg to 2.0 mg translates to 0.10 mg to 0.20 mg of modified Exendin-4 (active peptide agonist).

Data from the 3-month twice-weekly dosing study showed plasma concentrations of retro-Michael resistant albenatide (AB-013-AEEA-succinimide-albumin) increased after the initiation of twice weekly dosing in both dose groups. In the 2×1.5 mg (2×0.10 mg Ex4) dose group, plasma concentrations appeared to reach steady state by Day 22 and remained relatively constant over the rest of the treatment period. In the 2×1.5/2.0 mg (2×0.10/0.13 mg Ex4) dose group, plasma concentrations were highest on Day 22 with slightly lower levels observed on Day 50 and later. This observation is consistent with the dose reduction from 3.0 mg per week to 2.0 mg per week (0.20 mg per week to 0.13 mg per week Ex4) after the fourth week of dosing in this group. The mean steady-state Cpredose value on Day 78 for the antibody-negative participants in the 2×1.5/2.0 mg (2×0.10/0.13 mg Ex4) dose group was approximately 36% lower than the mean Cpredose value for the antibody-negative participants in the 2×1.5 mg (2×0.10 mg Ex4) dose group in the PP population. The mean (CV) t1/2 values of retro-Michael resistant albenatide (AB-013-AEEA-succinimide-albumin) in the 2×1.5 mg (2×0.10 mg Ex4) and 2×1.5/2.0 mg (2×0.10/0.13 mg Ex4) dose groups were 9.26 (20) days and 9.94 (31) days, respectively, for all participants in the PP population. For antibody-negative participants in the PP population, the mean (CV) t1/2 values of AB-013 were 9.25 (21) days and 9.19 (25) days in the 2×1.5 mg (2×0.10 mg Ex4) and 2×1.5/2.0 mg (2×0.10/0.13 mg Ex4) dose groups, respectively.

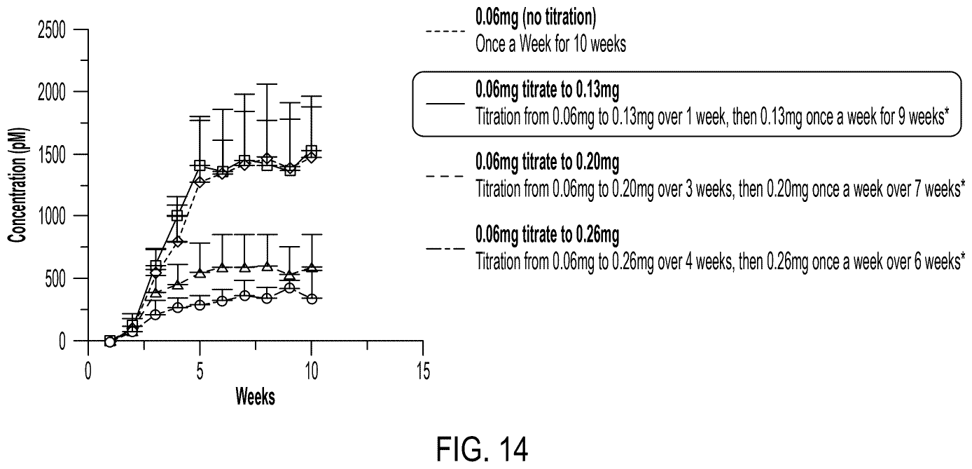

As can be seen from FIG. 13, a dose response effect can be observed from 0.06 mg q weekly to 0.10 mg q weekly to 0.13 mg q weekly to 0.20 mg q weekly with peak plasma concentration levels of retro-Michael resistant albenatide (AB-013-AEEA-succinimide-albumin) reaching>1,100 picomole. Once more, the indicated doses include the mass of albumin and the study was performed without q 4-week dose escalation titration. The doses of 1.0 mg to 3.0 mg translates to 0.06 mg to 0.20 mg of modified Exendin-4 (active peptide agonist). FIG. 14 shows results of another PK study in which subjects received retro-Michael resistant albenatide (AB-013-AEEA-succinimide-albumin) as follows: (a) 0.06 mg once a week for 10 weeks, no titration, (b) titration from 0.06 mg to 0.13 mg over 1 week, then 0.13 mg once a week over 9 weeks, (c) titration from 0.06 mg to 0.20 mg over 3 weeks, then 0.20 mg once a week over 7 weeks, and (d) titration from 0.06 mg to 0.26 mg over 4 weeks, then 0.26 mg once a week over 6 weeks. Here, it can be seen that the results are once more consistent with earlier studies and pharmacokinetic data, and that retro-Michael resistant albenatide (AB-013-AEEA-succinimide-albumin) achieved a low and flattened plasma profile.

It should further be noted that the differences in peak values between the studies support the evidence that in the presence of free-unbound agonist, a higher peak ensues. The absence of such material in the later study supports the evidence that fully albumin bound results in rapid transit of the molecule to the tissue compartment via the gp60 pathway. These findings furthermore support the evidence that fully bound albumin agonist participates provides a prolonged half-life of the GLP-1R agonist via not only through FcRn recycling but also via the transcytosis effect mediated through the endothelial cell receptor pathway of gp60, transporting the Albumin-bound molecule from the plasma compartment to the tissue compartment. The presence of fully-bound agonist with functional albumin resulting in rapid transcytosis of the albumin-bound agonist from the plasma compartment to the tissue compartment, thus contributes to the lower peak levels noted in this study. The difference in peak plasma values of unbound versus fully bound agonist is seen for both the 0.10 mg and the 0.20 mg doses, reflecting the biological potential of gp60/caveolin-1/caveolae transcytosis induced by albumin, transporting the molecule from the blood compartment to the tissue compartment. In conclusion, fully albumin-bound agonists may provide a lower peak plasma level at equivalent doses with steady state half-life and in so doing, may reduce the adverse events associated with rapid acute onset of peak plasma levels upon administration of the drug.

The Table below provides exemplary pharmacokinetic at 1 mg to 3 mg of retro-Michael resistant albenatide (AB-013-AEEA-succinimide-albumin).

| | 1.0 mg | 1.5 mg | 2.0 mg | 3.0 mg |
|---|---|---|---|---|
| | (0.065 mg) | (0.10 mg) | (0.13 mg) | (0.20 mg) |
| | (n = 18) | (n = 18) | (n = 23) | (n = 6) |
| Cmax (ng/mL) | 18.2 | 40.3 | 46.4 | 81.5 |
| Tmax (h) | 112.0 | 82.6 | 88.8 | 112.8 |
| AUC0-24 (ng · h/mL) | 138 | 500 | 577 | 1,146 |
| AUClast (ng · h/mL) | 4,778 | 13,209 | 15,654 | 27,277 |
| AUC0-inf (ng · h/mL) | 11,512 | 17,376 | 19,516 | 30,361 |
| AUCextra (%) | 25.6 | 21.5 | 19.8 | 11.2 |
| t½ (h) | 249.9 | 229.8 | 232.1 | 212.9 |
| kel (h−1) | 0.00325 | 0.00313 | 0.00307 | 0.00337 |
| MRT (h) | 385.4 | 355.7 | 367.4 | 335.2 |
| Cl/F (mL/min) | 1.48 | 1.55 | 1.82 | 1.86 |
| Vd/F (L) | 33.8 | 31.2 | 37.1 | 34.9 |

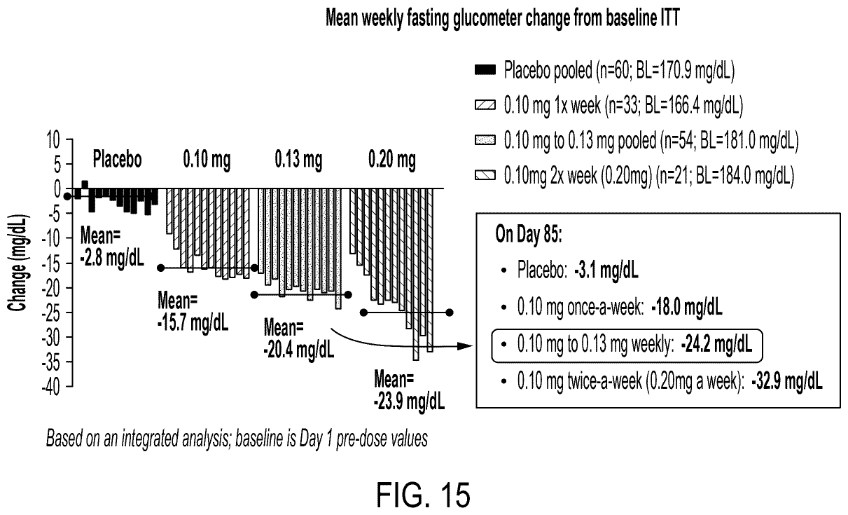

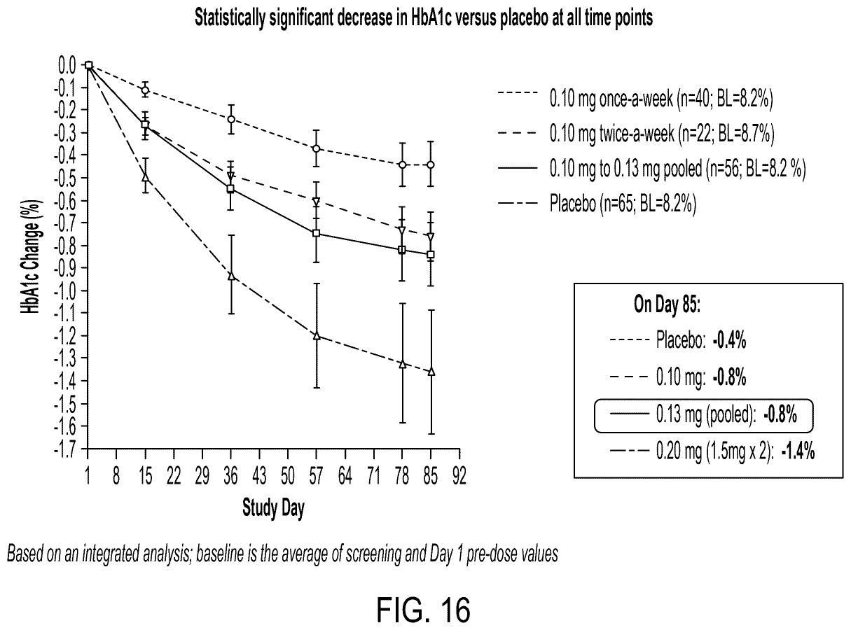

The inventors further set out to determine whether contemplated compounds, and in particular the retro-Michael resistant albenatide (AB-013-AEEA-succinimide-albumin) would be effective in human subjects at a low dose (here: 130 mcg of agonist peptide) to reduce fasting blood glucose and HbA1c upon administration of the compound. To that effect, the inventors analyzed that glucose and HbA1c from Type 2 diabetic subjects that had received placebo, 0.1 mg once a week, 0.1 mg to 0.13 mg once a week, and 0.1 mg twice a week over a period of 85 days, and exemplary results are shown in FIG. 15. As can be clearly taken from FIG. 15, administration of the retro-Michael resistant albenatide had a statistically significant and dose-dependent effect of the reduction of fasting blood glucose. Indeed, subjects receiving 0.1 mg weekly had a reduction in blood glucose of about 18 mg/dl, subjects receiving 0.1-0.13 mg weekly had a reduction in blood glucose of about 20.4 mg/dl, and subjects receiving 0.1 mg twice weekly had a reduction in blood glucose of about 23.9 mg/dl on day 85. Similarly, when looking at a reduction in HbA1c over 85 days in Type 2 diabetic subjects, the inventors noted that subjects receiving 0.1 mg weekly had a reduction in HbA1c of about 0.8%, subjects receiving 0.1-0.13 mg weekly had a reduction in HbA1c of about 0.8%, and subjects receiving 0.1 mg twice weekly had a reduction in HbA1c of about 1.4%, on day 85. Thus, therapeutic efficacy at a very low dose was achieved.

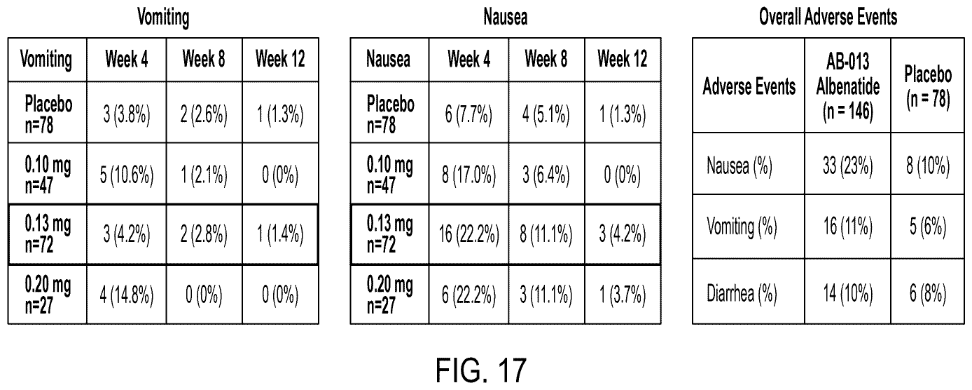

Even more advantageously, the inventors discovered that the same group of subjects experienced a substantially lowered rate of adverse events, and particularly a reduced incidence of vomiting and nausea, indicating that contemplated retro-Michael resistant compounds have a very high therapeutic index. As can be readily seen from the exemplary results in FIG. 17, the retro-Michael resistant albenatide (AB-013-AEEA-succinimide-albumin) was well tolerated at the 0.13 mg dose. Indeed, base don the data sown in FIG. 17 the inventors contemplated that the dose can be increased via a titration program to 0.2 mg at week 12.

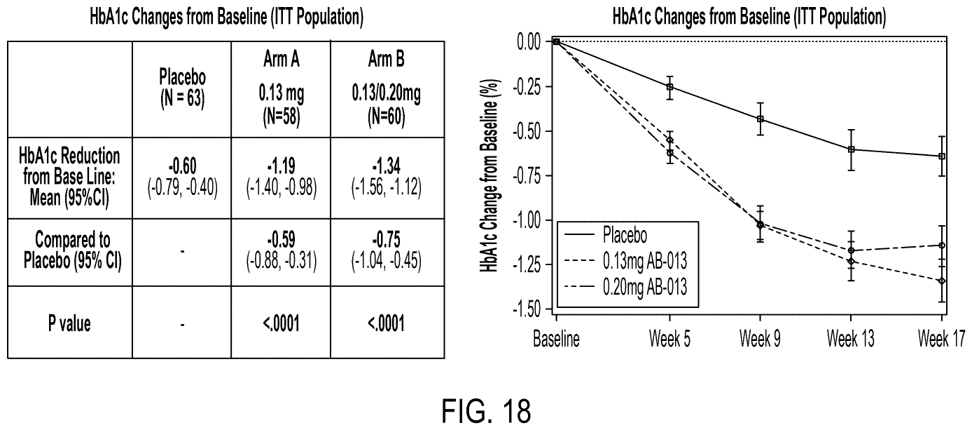

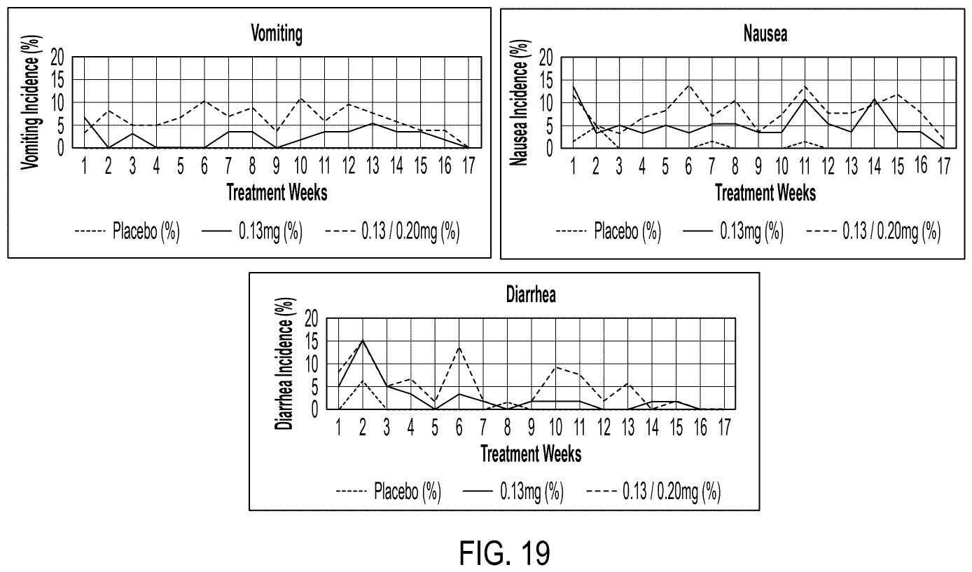

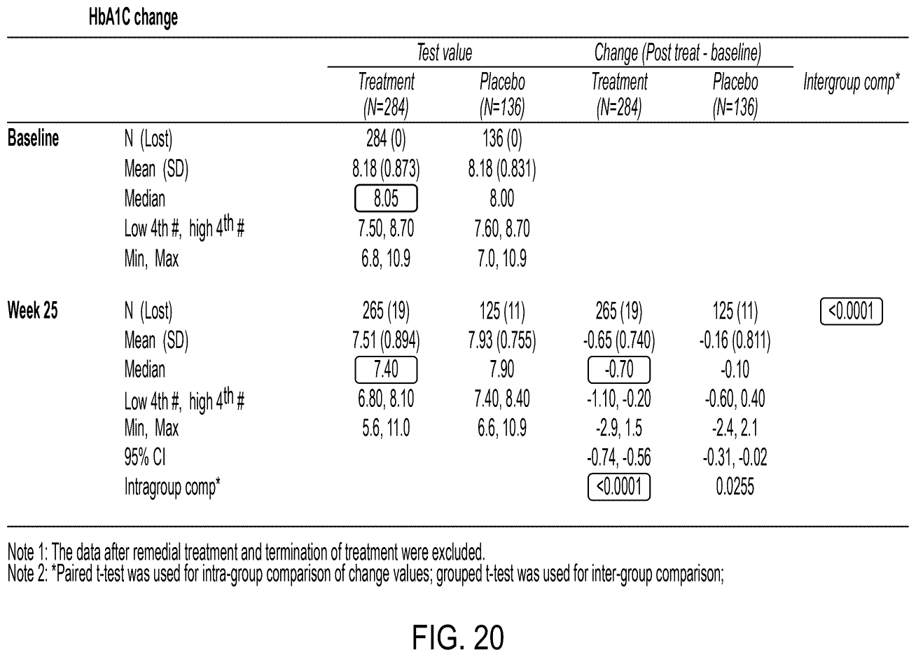

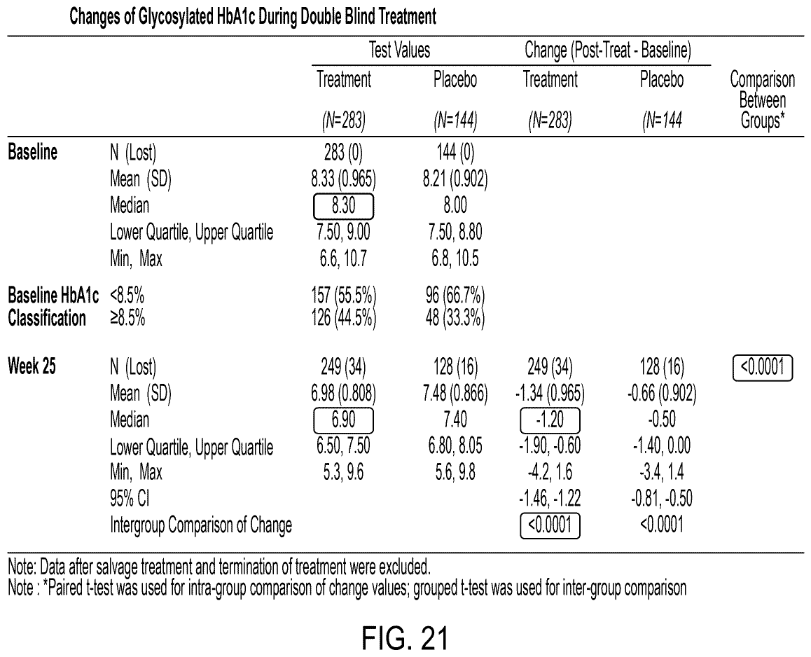

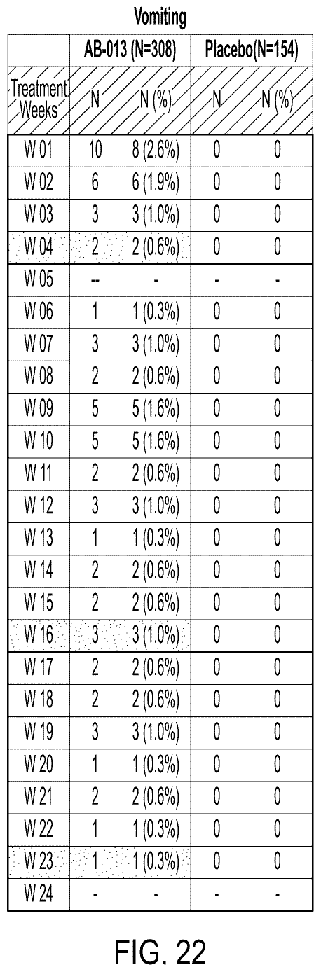

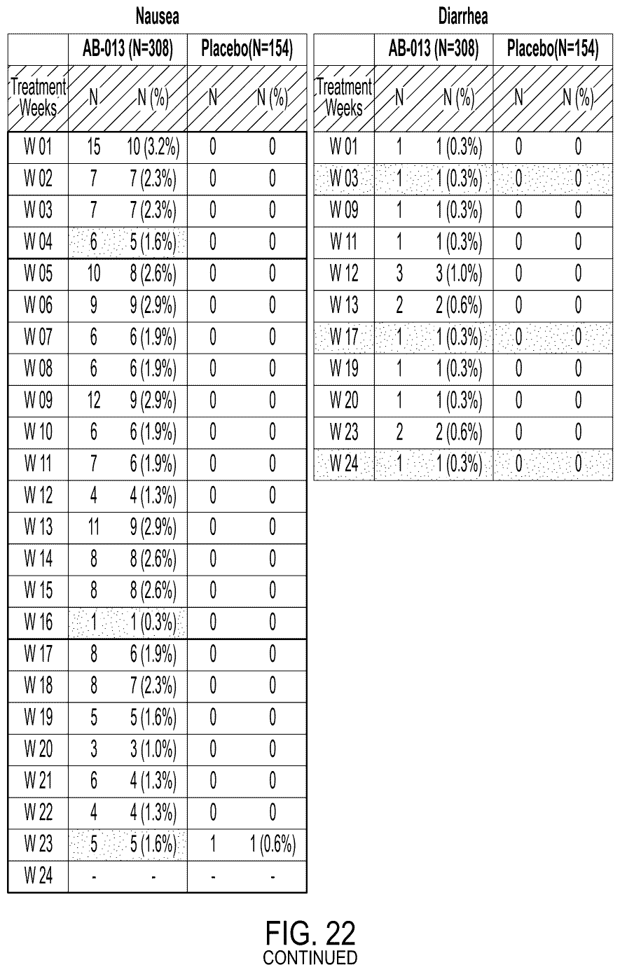

In additional studies extending up to 25 weeks in subjects with Type 2 diabetes with metformin therapy, the inventors once mor found that low dosages of the retro-Michael resistant albenatide (AB-013-AEEA-succinimide-albumin) at 0.13 mg or 0.20 mg consistently lowered HbA1c in a statistically significant manner at very low adverse effect rates as is exemplarily shown in FIG. 18. Here, an ultra-low dose of 0.13 mg of retro-Michael resistant albenatide (AB-013-AEEA-succinimide-albumin) resulted in a significant HbA1c reduction by week 5, and a reduction of >1% by Week 9. Such findings, along with the substantially lowered blood glucose in earlier studies confirms the hypothesis that the irreversibly albumin-bound Exendin-4 binds effectively to the GLP-1 receptor with a therapeutic effect at an ultralow dose. In that context, it should be noted that a comparable effect with semaglutide (reversibly associated exendin-4) requires a substantially higher dosage of at least 2.4 mg exendin-4, leading to significantly higher rates adverse effects due to the unbound exendin-4 concentration in plasma. FIG. 19 depicts exemplary results for adverse effects in the same patient population. As can be readily seen from FIG. 19, GI adverse events were less than <5% by week 9 (vomiting, nausea, and diarrhea) in subjects receiving 0.13 mg without titration and sustained this reduction to the end of the study at Week 17. At this ultra-low dose, HbA1C decrease by more than 1.0%. FIG. 20 and FIG. 21 depict results of two further studies in which Type 2 diabetes patients on metformin or metformin-naïve, respectively, received once weekly 0.13 mg doses of retro-Michael resistant albenatide (AB-013-AEEA-succinimide-albumin) without titration. Here, at week 25, subjects with Type 2 diabetes patients on metformin had a reduction of HbA1c of 0.7%, while subjects with Type 2 diabetes patients (metformin-naïve) had a reduction of HbA1c of 1.2%. FIG. 22 depicts adverse events after week 24 for these study populations. Once more, it should be appreciated that at the ultra-low dose of 0.13 mg without titration, the GI adverse events were minimal with less than 2% by week 4 for vomiting, nausea, and diarrhea.

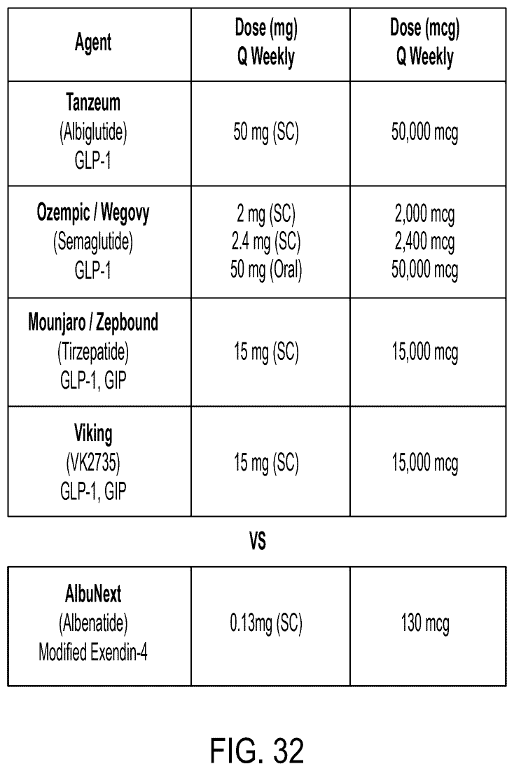

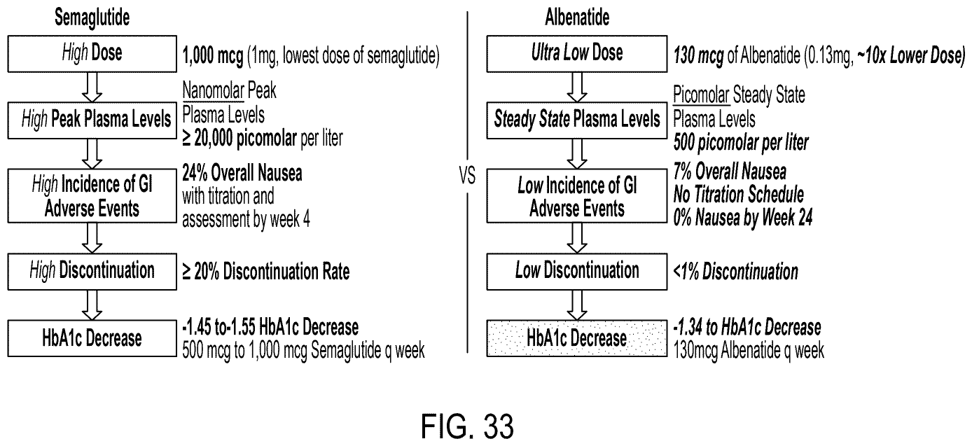

FIG. 32 is illustrative of the unexpected advantages of contemplated compounds, and particularly of retro-Michael resistant fusion proteins, such as retro-Michael resistant albenatide (AB-013-AEEA-succinimide-albumin). Heretofore it was believed that only the free fraction of a peptide agonist in the plasma that is not bound to albumin would be available to activate GLP-1, GIP, and/or Glucagon Receptors, and it was believed that albumin-bound GLP-1 is not available for binding to a receptor in an effective manner (or at all). Indeed, albugiptide had activity only at ultra-high quantities of the peptide agonist (50,000 mcg), while other compositions in which the peptide agonist was non-covalently associated with albumin administered quantities were about 15,000 mcg for some injected forms, while semaglutide still required 2,400 mcg at the lowest administered dose. In contrast, retro-Michael resistant albenatide (AB-013-AEEA-succinimide-albumin) provides about 130 mcg per dosage of the modified exendin-4, rendering the retro-Michael resistant fusion protein much more tolerable while maintaining therapeutic effect. FIG. 33 illustrates the same situation in more detail, comparing semaglutide administration with retro-Michael resistant albenatide (AB-013-AEEA-succinimide-albumin) administration. As can be readily taken from FIG. 33, administration of retro-Michael resistant albenatide required for steady-state plasma levels and zero nausea by the end of the study with similar drops in HbA1c a dose that is about 10 times lower that the lowest dose for semaglutide.

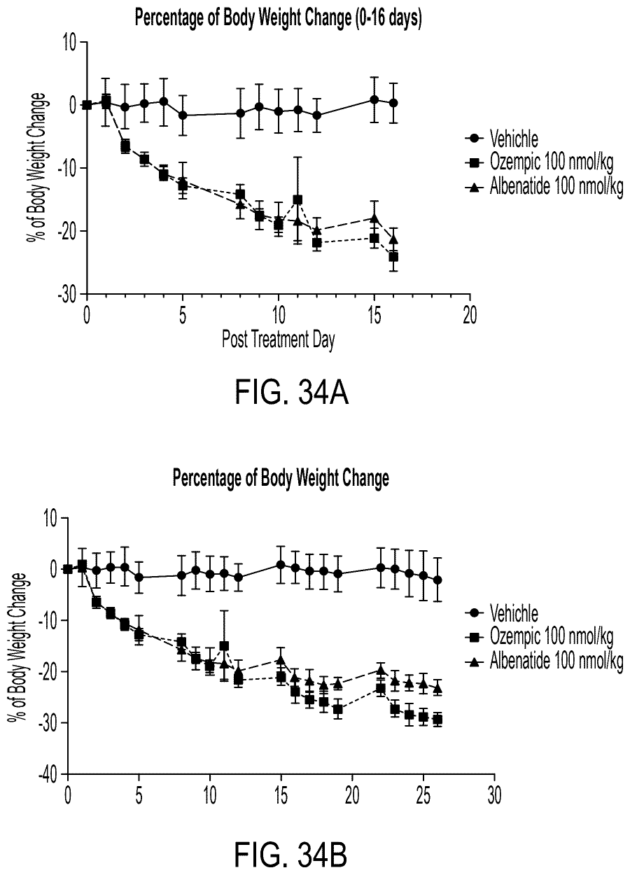

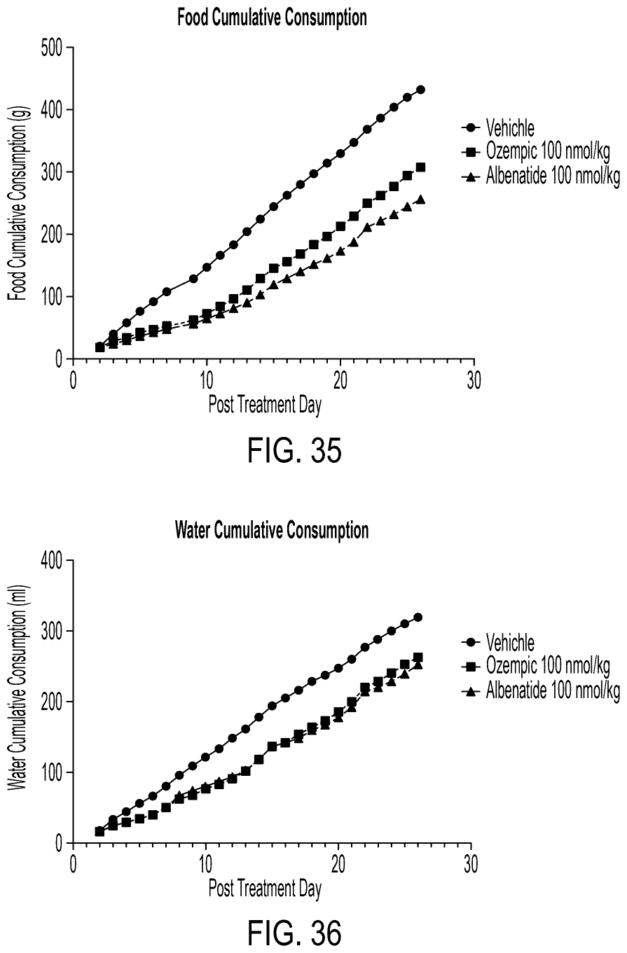

In still further experiments to confirm that contemplated compounds presented herein are also effective as modulators for hepatic metabolism, and particularly in the treatment and even at least partial reversion of NASH (non-alcoholic steatohepatosis), the inventors tested selected compounds in DIO (diet induced obesity) mice. More particularly, DIO mice received a subcutaneous injection of either semaglutide (OZEMPIC™, NovoNordisk) or retro-Michael resistant albenatide (AB-013-AEEA-succinimide-albumin) at a single dosage of 100 nmol/kg. The body weight of all treated animals was recorded over 26 days, and average body weight over time for 16 and 26 days are shown in FIG. 34A and FIG. 34B, respectively. AS can be readily seen, both tested compounds were effective in reduction of body weight, which is also reflected in FIG. 35 showing results for cumulative food consumption and FIG. 36 showing results for cumulative water consumption.

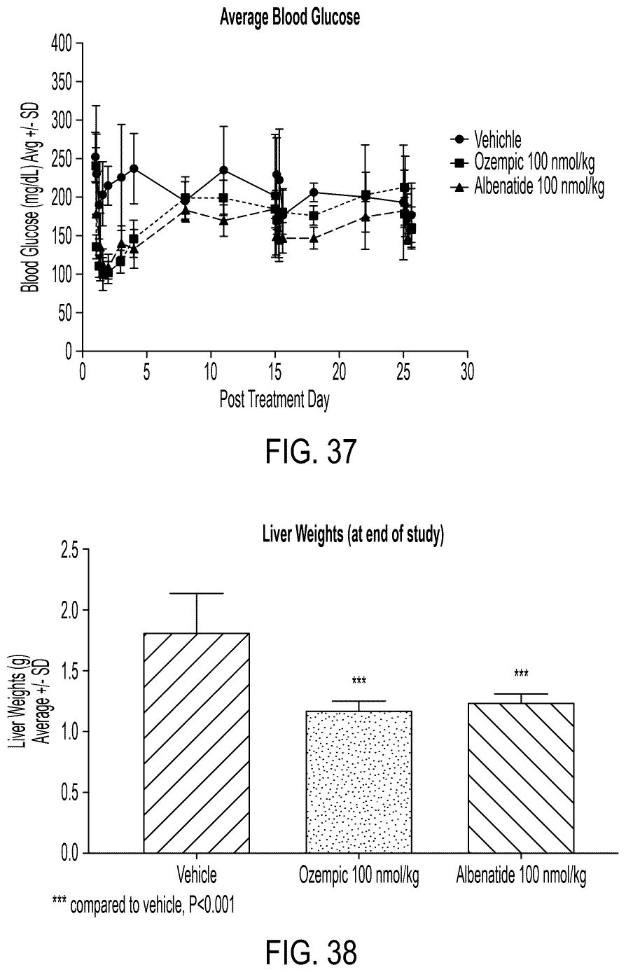

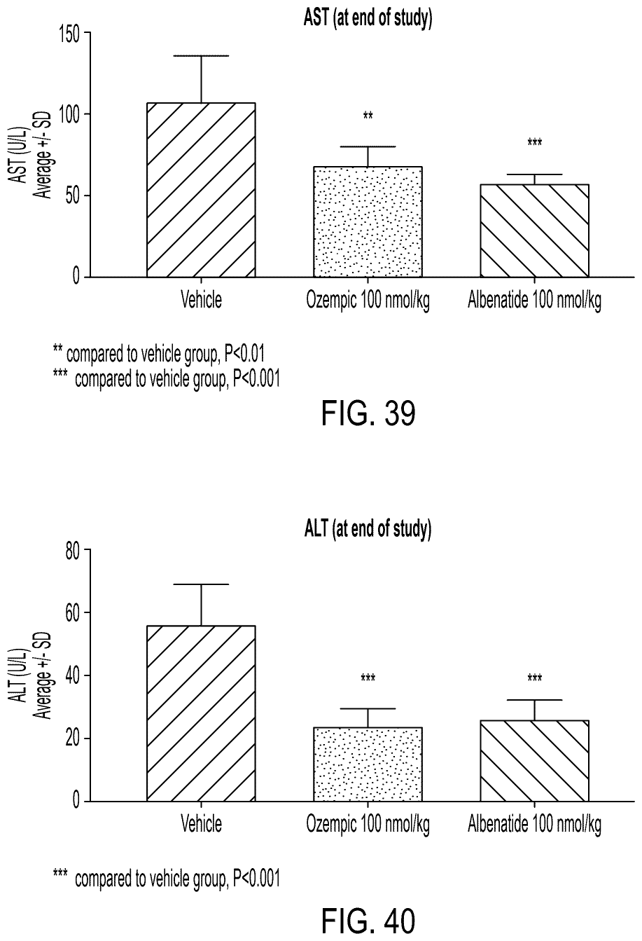

Blood glucose was followed over the entire test period, and as can be seen from the data in FIG. 37, the blood glucose level was substantially reduced post injection and trended towards control over time. To investigate liver function and metabolism, liver weight was determined at the end of the study and results are shown in FIG. 38. As can be readily seen from FIG. 38, liver weight was significantly reduced in both test groups versus placebo, indicating a reduction in hepatic lipids. In addition, liver function was assessed at the end of the study by testing AST (aspartate aminotransferase) and ALT (alanine transaminase). Notably, as shown in FIG. 39 (AST) and FIG. 40 (ALT), both liver enzymes indicated a significant improvement over control, once more demonstrating the hepatoprotective effect of retro-Michael resistant albenatide (AB-013-AEEA-succinimide-albumin).

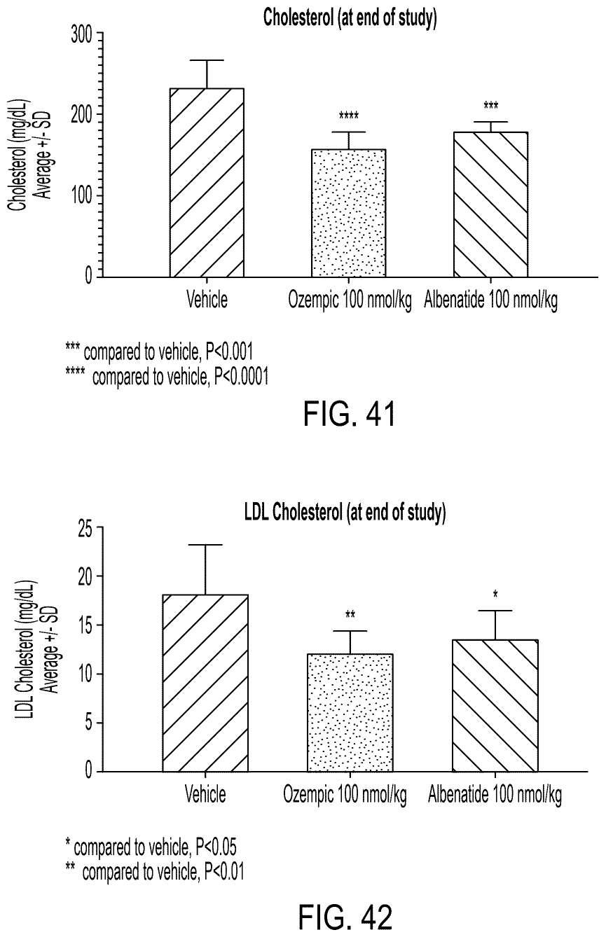

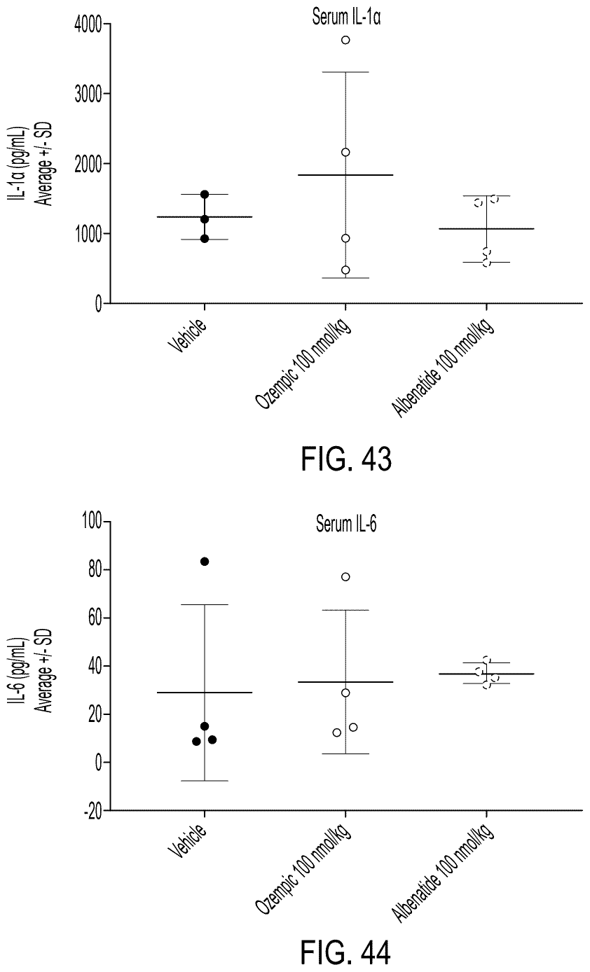

In still further tests for hepatic metabolic modulation, the inventors also determined total cholesterol and LDL cholesterol at the end of the study, and exemplary results are shown in FIG. 41 (for total cholesterol) and FIG. 42 (for LDL cholesterol). Remarkably here as well, total cholesterol and LDL cholesterol were substantially reduced after administration of only a single dose. As diet induced obesity is also often associated with inflammation, the inventors also measured levels of selected pro-inflammatory cytokines. Here, IL-1α and IL-6 levels were determined at the end of the study. Interestingly, in animals treated with retro-Michael resistant albenatide (AB-013-AEEA-succinimide-albumin), both measured pro-inflammatory cytokines were tightly grouped as compared to the semaglutide treatment group, suggesting an anti-inflammatory effect even 25 days post injection.

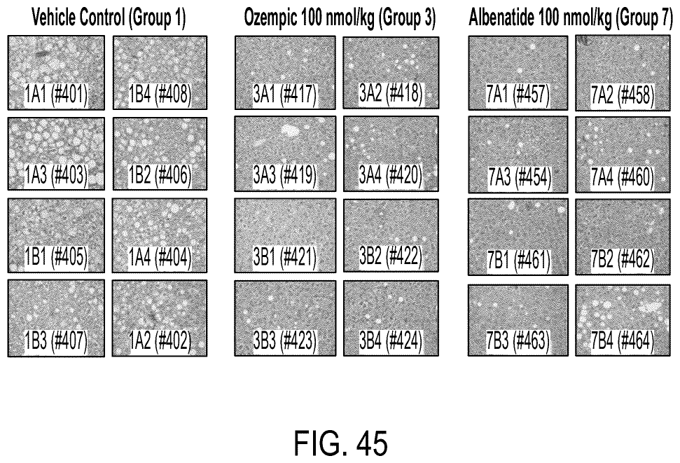

Finally, to confirm that the blood lipid and liver weight reduction by retro-Michael resistant albenatide (AB-013-AEEA-succinimide-albumin) treated animals was due to hepatic lipid loss, the livers of the mice were sectioned and stained with hematoxylin and eosin. AS can be readily seen form the photomicrographs in FIG. 45, intracellular lipid droplets were abundantly present in the control animals whereas in the treatment group with retro-Michael resistant albenatide (AB-013-AEEA-succinimide-albumin), hepatic lipid accumulation was substantially reduced. Therefore, it should be recognized that contemplated compounds, and especially retro-Michael resistant albenatide (AB-013-AEEA-succinimide-albumin) is effective in halting progression of, and even at least partially reversing NASH.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID No:1-471 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on: —(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants. Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance. Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, suitable calculations of the percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100. Alternative methods for identifying similar sequences will be known to those skilled in the art. For example, a substantially similar nucleotide sequence will be encoded by a sequence which hybridizes to DNA sequences or their complements under stringent conditions. By stringent conditions, we mean the nucleotide hybridizes to filter-bound DNA or RNA in 3× sodium chloride/sodium citrate (SSC) at approximately 45° C. followed by at least one wash in 0.2×SSC/0.1% SDS at approximately 20-65° C.

Alternatively, a substantially similar polypeptide may differ by at least 1, but less than 5, 10, 20, 50 or 100 amino acids from the sequences shown in, for example, in the amino acid sequence that are included within SEQ ID Nos: 1-471. Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of treating a subject, comprising:
administering a therapeutically effective amount of a pharmaceutical composition to the subject in need thereof;
wherein the pharmaceutical composition comprises a fusion protein in which a mono-agonist peptide having binding and activation specificity for a G protein-coupled receptor (GPCR) selected from the group consisting of a glucagon-like peptide 1 (GLP-1) receptor, a glucose-dependent insulinotropic polypeptide (GIP) receptor, and a glucagon (GcG) receptor, is covalently coupled to a Cys₃₄ amino acid of a conformationally modified albumin, wherein the albumin has no hydrophobic ligands in any hydrophobic fatty acid binding pockets, wherein the covalent coupling creates a stereopreferred or stereoselective chirality center; and
wherein the pharmaceutical composition is substantially free of unbound agonist peptide.

2. The method of claim 1, wherein the therapeutically effective amount is equal to or less than 1,000 mcg of the GPCR agonist peptide orally administered once per day.

3. The method of claim 1, wherein the composition, upon administration, reduces blood glucose, reduces HbA1c, and/or reduces hepatic lipid accumulation.

4. The method of claim 1, wherein the subject is diagnosed with a metabolic disorder selected from the group consisting of NIDDM (non-insulin dependent diabetes mellitus), NASH (non-alcoholic steatohepatosis/steatohepatitis), and obesity.

5. The method of claim 1, wherein the subject is diagnosed with a neurodegenerative disease selected from the group consisting of Parkinson's Disease, Alzheimer's Disease, peripheral neuropathy, multiple sclerosis, and amyotrophic lateral sclerosis (ALS).

6. The method of claim 1, wherein the subject is diagnosed with an autoimmune or inflammatory disease selected from the group consisting of Crohn's disease, irritable bowel syndrome (IBS), ulcerative colitis, and polycystic ovary syndrome.

7. The method of claim 1, wherein the GPCR agonist peptide is a GLP-1 mono-agonist peptide.

8. The method of claim 1, wherein the GPCR agonist peptide is a GIP mono-agonist peptide.

9. The method of claim 1, wherein the GPCR agonist peptide is a GcG mono-agonist peptide.

10. A method of treating a subject, comprising:
administering a therapeutically effective amount of a pharmaceutical composition to the subject in need thereof;
wherein the pharmaceutical composition comprises a fusion protein in which a dual-agonist peptide having binding and activation specificity for two G protein-coupled receptors (GPCR) selected from the group consisting of a glucagon-like peptide 1 (GLP-1) receptor, a glucose-dependent insulinotropic polypeptide (GIP) receptor, and a glucagon (GcG) receptor, is covalently coupled to a Cys₃₄ amino acid of a conformationally modified albumin, wherein the albumin has no hydrophobic ligands in any hydrophobic fatty acid binding pockets, wherein the covalent coupling creates a stereopreferred or stereoselective chirality center; and
wherein the pharmaceutical composition is substantially free of unbound agonist peptide.

11. The method of claim 10, wherein the therapeutically effective amount is equal to or less than 1,000 mcg of the GPCR agonist peptide orally administered once per day.

12. The method of claim 10, wherein the composition, upon administration, reduces blood glucose, reduces HbA1c, and/or reduces hepatic lipid accumulation.

13. The method of claim 10, wherein the subject is diagnosed with a metabolic disorder selected from the group consisting of NIDDM (non-insulin dependent diabetes mellitus), NASH (non-alcoholic steatohepatosis/steatohepatitis), and obesity.

14. The method of claim 10, wherein the subject is diagnosed with a neurodegenerative disease selected from the group consisting of Parkinson's Disease, Alzheimer's Disease, peripheral neuropathy, multiple sclerosis, and amyotrophic lateral sclerosis (ALS).

15. The method of claim 10, wherein the subject is diagnosed with an autoimmune or inflammatory disease selected from the group consisting of Crohn's disease, irritable bowel syndrome (IBS), ulcerative colitis, and polycystic ovary syndrome.

16. The method of claim 10, wherein the GPCR agonist peptide is a GLP-1/GIP dual-agonist peptide.

17. The method of claim 10, wherein the GPCR agonist peptide is a GLP-1/GcG dual-agonist peptide.

18. The method of claim 10, wherein the GPCR agonist peptide is a GIP/GcG dual-agonist peptide.

19. A method of treating a subject, comprising:
administering a therapeutically effective amount of a pharmaceutical composition to the subject in need thereof;
wherein the pharmaceutical composition comprises a fusion protein in which a tri-agonist peptide having binding and activation specificity for a glucagon-like peptide 1 (GLP-1) receptor, a glucose-dependent insulinotropic polypeptide (GIP) receptor, and a glucagon (GcG) receptor, is covalently coupled to a Cys₃₄ amino acid of a conformationally modified albumin, wherein the albumin has no hydrophobic ligands in any hydrophobic fatty acid binding pockets, wherein the covalent coupling creates a stereopreferred or stereoselective chirality center; and
wherein the pharmaceutical composition is substantially free of unbound agonist peptide.

20. The method of claim 19, wherein the therapeutically effective amount is equal to or less than 1,000 mcg of the GPCR agonist peptide orally administered once per day.

21. The method of claim 19, wherein the composition, upon administration, reduces blood glucose, reduces HbA1c, and/or reduces hepatic lipid accumulation.

22. The method of claim 19, wherein the subject is diagnosed with a metabolic disorder selected from the group consisting of NIDDM (non-insulin dependent diabetes mellitus), NASH (non-alcoholic steatohepatosis/steatohepatitis), and obesity.

23. The method of claim 19, wherein the subject is diagnosed with a neurodegenerative disease selected from the group consisting of Parkinson's Disease, Alzheimer's Disease, peripheral neuropathy, multiple sclerosis, and amyotrophic lateral sclerosis (ALS).

24. The method of claim 19, wherein the subject is diagnosed with an autoimmune or inflammatory disease selected from the group consisting of Crohn's disease, irritable bowel syndrome (IBS), ulcerative colitis, and polycystic ovary syndrome.

25. A method of treating a subject, comprising:
orally administering a therapeutically effective amount of a pharmaceutical composition to the subject in need thereof;
wherein the pharmaceutical composition comprises a fusion protein in which an agonist peptide having binding and activation specificity for one or more G protein-coupled receptors (GPCR) selected from the group consisting of a glucagon-like peptide 1 (GLP-1) receptor, a glucose-dependent insulinotropic polypeptide (GIP) receptor, and a glucagon (GcG) receptor, is covalently coupled to a Cys₃₄ amino acid of a conformationally modified albumin, wherein the albumin has no hydrophobic ligands in any hydrophobic fatty acid binding pockets, wherein the covalent coupling creates a stereopreferred or stereoselective chirality center; and
wherein the pharmaceutical composition is substantially free of unbound agonist peptide.

26. A method of treating a subject, comprising:
administering once per day a therapeutically effective amount of a pharmaceutical composition to the subject in need thereof;
wherein the pharmaceutical composition comprises a fusion protein in which an agonist peptide having binding and activation specificity for one or more G protein-coupled receptors (GPCR) selected from the group consisting of a glucagon-like peptide 1 (GLP-1) receptor, a glucose-dependent insulinotropic polypeptide (GIP) receptor, and a glucagon (GcG) receptor, is covalently coupled to a Cys₃₄ amino acid of a conformationally modified albumin, wherein the albumin has no hydrophobic ligands in any hydrophobic fatty acid binding pockets, wherein the covalent coupling creates a stereopreferred or stereoselective chirality center; and
wherein the pharmaceutical composition is substantially free of unbound agonist peptide.
